(19) United States
(12) Patent Application Publication
(10) Pub. No.: US 2012/0263649 A1
(43) Pub. Date: Oct. 18, 2012

(54) DETECTION OF MYCOBACTERIA

(75) Inventors: Keriann Marie Backus, Oxford (GB); Benjamin Davis, Oxford (GB); Clifton B. Barry, III, Germantown, MD (US); Helena Boshoff, Potomac, MD (US)

(73) Assignee: ISIS INNOVATION LIMITED, Oxford (GB)

(21) Appl. No.: 13/395,382

(22) PCT Filed: Sep. 10, 2010
(86) PCT No.: PCT/GB2010/051519

§ 371 Date: Jun. 25, 2012

Related U.S. Application Data
(60) Provisional application No. 61/241,741, filed on Sep. 11, 2009.

(51) Int. Cl. C07H 5/06; A61K 49/00; G01N 24/08; C07H 3/04; C12Q 1/04; G01N 21/64

(52) U.S. Cl. 424/9.1; 435/34; 536/123.13; 536/55

(57) ABSTRACT

A method for determining the presence of mycobacteria species in an organism or biological sample, the method comprising adding to the organism or biological sample a probe molecule comprising a substrate and a label, which probe molecule can be incorporated into mycobacteria, the presence of mycobacteria being determined by a detector responsive to the presence of the label, optionally after applying a stimulus; suitable probe molecules include compounds comprising a label and a substrate, which label is can be detected by a detector responsive to the presence of the label, optionally after applying a stimulus, characterised by compound being able to engage with the active site of Antigen 85B (Ag85B) such that it can form simultaneous hydrogen bonds with two or more amino acids in the active site selected from Arg 43, Trp 264, Ser126, His 262 and Leu 42, or the corresponding amino acids in Antigen 85A (Ag85A) or Antigen 85C (Ag85C), at least one of which is with Ser126.

This invention was made with United States Government support. The United States Government has certain rights in the invention.

This invention relates to detection of mycobacteria, more specifically to a method for attaching a detectable label to mycobacteria and compounds for use in such a method.

Tuberculosis (TB) is an infection that has plagued mankind for millennia, and remains a leading cause of death worldwide. The bacteria responsible for infections such as tuberculosis in humans and corresponding infections in other animal species, are species of Mycobateria. These bacteria are often difficult to eradicate due to the nature of their cell envelope, which provides a significant permeability barrier.

An obstacle to the development of new diagnostics, drugs and vaccines is the lack of bacteria-specific probes that can be used to assess total burden of disease in infected patients.

One of the characteristic features of Mycobacteria, for example M. Tuberculosis (Mtb), is their synthesis of a non-mammalian sugar, trehalose (Tre) through three independent pathways, the blocking of any of which can result in bacterial death or growth defects.

Trehalose is found in the outermost portion of the mycobacterial membrane, along with the glycolipids trehalose dimycolate (TDM) and trehalose monomycolate (TMM). These are important glyco lipids in Mtb, because they induce granuloma formation in an infected subject.

Mycolic acids are also present in mycobacterial membranes. These are long chain (C30 to C90) cyclopropanated lipids which are important in mycobacterial membrane structure, virulence and persistence within a host. Tre is incorporated into the mycobacterial cell wall in the form of esters of mycolic acid by the action of the extracellular enzymes, antigens 85A, 85B and 85C, henceforth Ag85A, B or C respectively, (Kilburn et al; Biochemical and biophysical research communications 108 (1), 132 (1982)), which catalyse the reversible transesterification reaction between two TMM units to generate TDM and free Tre. The reverse reaction allows for direct esterification of Tre to TMM. It has been found that knocking-out genes coding for single members of the Ag85 proteins have significant effects on mycolic acid incorporation (Harth et al; Proc Natl Acad Sci USA 99 (24), 15614 (2002)). Additionally, Ag85 enzymes can also covalently introduce mycolates into arabinogalactan cell wall polymers (Jackson, M. et al; Mol Microbiol 31 (5), 1573 (1999); Puech, V. et al; Mol Microbiol 35 (5), 1026 (2000)).

The highly infectious and transmittable nature of mycobacteria such as Mtb means that fast and effective diagnosis is important. Examples of tests for detecting Mtb are summarised in Health Technology Assessment, 2007, Volume 11(3), pages 4 to 9. Typically, they involve testing a specimen, for example sputum, cerebrospinal fluid, pericardial fluid, synovial fluid, ascitic fluid, blood, bone marrow, urine and faeces.

Analysis of specimens by microscopy is one of the most rapid techniques available, although is not necessarily accurate.

Culture-based techniques are more sensitive, but are comparatively slow because of the low rate of mycobacterial growth, often up to 6 to 8 weeks.

Sereological tests have been developed, to detect the appearance of certain antibodies in the blood. However, although such methods are generally low cost and rapid, they tend to suffer from poor sensitivity. Additionally, they can suffer from poor accuracy due to antibody responses often being non-specific to mycobacteria antigens.

Use of biochemical markers have also been attempted, for example the analysis of adenosine deaminase in lymphocites, and analysis of cytokines such as interferon-γ and TNF-α. However, such tests also suffer from a lack of specificity to mycobacteria. Blood samples can be tested by adding specific mycobacterial antigens and detecting interferon-γ produced by lymphocytes. However, this often requires significant handling and processing of the blood to isolate mononuclear cells, which handling and processing must be done within in a short period of time, typically less than 12 hours from collection of the blood sample.

Nucleic amplification tests are available, in which DNA or rRNA from a micro-organism is amplified using reactions such as the polymerase chain reaction or ligase chain reaction. However, because different mycobacteria have different genetic make-up, such tests are generally only reliable for individual species.

Mycobacteriophage methods are known, in which mycobacteria are infected with a phage, exogeneous non-infecting phage killed, and any infecting phage that is amplified through reproduction in the mycobacteria is detected. One test involves use of a luciferase reporter phage, which produces quantifiable light. However, such phage-based methods often require mycobacterial cultures, with the consequent disadvantages associated therewith.

Known ways of imaging mycobacteria in infected macrophages do not allow non-toxic imaging of bacteria in vivo (Beatty, W. L. et al; Traffic 1 (3), 235 (2000); Beatty et al; Infect. Immun. 68 (12), 6997 (2000); Thanky et al; Tuberculosis (Edinb) 87 (3), 231 (2007)). Additionally, attempts at chemically labelling mycobacteria, for example with texas red by oxidation with sodium periodate, suffer from lack of selectivity, and also can potentially cause damage to the bacterial cell wall. Fluorescently labelled vancomycin has been used to track cell division patterns of M. Smegmatis and the BCG vaccine, based on M. bovis, but it is toxic to other mycobacteria such as Mtb, which limits its use for detection thereof.

Tests that can be carried out directly on a human or animal body include the tuberculin skin test, which injects small quantities of a number of antigens shared by several mycobacteria, the presence of which gives rise to a skin reaction. However, this requires two separate visits to a practitioner, one for the injection of the antigens, and another within 48-72 hours for detection of the skin reaction. Additionally, there are further disadvantages such as difficulties associated with test administration and interpretation, the potential for painful skin inflammation and scarring, and also preclusion of the test for people with certain skin disorders.

In view of the problems associated with existing methods of detection, there remains a need for an alternative method of detecting mycobacteria.

According to the present invention, there is provided a method for determining the presence of mycobacteria species in an organism or biological sample, the method comprising adding to the organism or biological sample a probe molecule comprising a substrate molecule and a label, which probe molecule can be incorporated into mycobacteria, the presence of mycobacteria being determined by a detector responsive to the presence of the label, optionally after applying a stimulus.

The inventors have found that mycobacteria can be modified by incorporating into their structure a probe molecule comprising a substrate molecule and a label, which label can be detected directly or after applying a stimulus.

The probe molecule is able to be incorporated into mycobacteria. In a preferred embodiment, the probe molecule is able to be chemically incorporated into the mycobacterial cell wall. This can be achieved by making use of one or more of the Ag85A, B and C enzymes that are common to mycobacteria, which incorporate the probe molecule into the mycolic acid layer of a mycobacterial cell wall. Although Ag85A, B and C typically require Tre or its mycolic acid esters as the substrate, the inventors have found that the Ag 85A, B and C enzymes can be relatively non-specific, and do not necessarily require the substrate to conform exactly to the Tre structure in order to become esterified or transesterified with mycolate. Therefore, the inventors have been able to produce probe molecules based on labelled substrates, often with significantly large labels, which are able to be reactants in the Ag 85 A, B and C catalysed reactions, enabling the probe molecule to become chemically incorporated into mycobacterial cell walls. Additionally, probe molecules have been designed which have a reactive functional group which can react with a Serine residue present in the active site of Ag85A, B and C, which chemically binds the probe molecule to the enzyme.

The probe molecule comprises a substrate and a label. Examples of labels include luminescent labels which emit radiation on exposure to an external source of radiation or other stimulus, e.g. fluorescent materials or fluorophors (which emit light when exposed to light), chemiluminescent materials (which emit light during chemical reaction), electroluminescent materials (which emit light on application of an electric current), phosphorescent materials (in which emission of light continues after exposure to light stimulus has ended) and thermoluminescent materials (which emit light once a certain temperature is exceeded). Fluorophors are often used, and examples of molecular families that can act as fluorophors include fluoresceins, xanthenes, cyanines, naphthalenes, coumarins, oxadiazoles, pyrenes, oxazines, acridines, arylmethines, Alexa Fluors and tetrapyrroles. Further fluorophors include quantum dots, which emit highly specific wavelengths of electromagnetic radiation after stimulation, for example by electricity or light.

Other labels include radioactive labels, including positron emitting nuclei such as ¹⁸F, ⁶⁴Cu or ¹²⁴I which can be detected by imaging techniques such as positron emission topography (PET). Other radioactive labels such as ¹⁴C, ³H, or iodine isotopes such as ¹²³I and ¹³¹I, which can be detected using autoradiographic analysis or scintillation detection for example, can also be used. In the case of gamma-emitting nuclei, imaging techniques such as single photon emission computed tomography (SPECT) can be used. Other labels include those that are NMR-active, which can be detected by magnetic resonance techniques, for example magnetic resonance imaging (MRI) or nuclear magnetic resonance (NMR) detectors, the labels typically comprising one or more NMR-active nuclei that are not generally found in concentrated form elsewhere in the organism, biological sample or mycobacterium, examples being ¹³C, ²H (deuterium) or ¹⁹F. Further labels include those comprising atoms with large nuclei, for example atoms with atomic number of 35 or more, preferably 40 or more and even more preferably 50 or more, for example iodine or barium, which are effective contrast agents for X-ray photographic techniques or computed tomography (CT) imaging techniques.

Other labels include those that can bind favourably and specifically to another reagent, for example use of a biotin label. Biotin binds very specifically to avidin and streptavidin, and hence the presence of a biotin label can be detected by addition of an avidin or streptavidin-modified molecule, for example avidin and streptavidin-modified fluorescent dyes.

In one embodiment, the label is a molecule that is chemically attached to a substrate molecule to form the probe molecule. Labelling a substrate molecule to form a probe molecule can be achieved by derivatising the substrate molecule with a chemical group that can react with a corresponding group on a label molecule to produce a chemical bond that chemically links the substrate and label molecules to produce the probe molecule. Henceforth such derivative groups are termed linking groups. Thus the substrate molecule and/or the label molecule before their chemical combination can comprise one or more linking groups which react together to form a covalent bond between the label and substrate. For example, to attach a label comprising a fluorescein group to a mono- or disaccharide substrate molecule, the fluorescein is often modified with an isothiocyanate linking group, namely fluorescein isothiocyanate (FITC), which is then reacted with an amine linking group present on a modified mono- or disaccharide probe molecule resulting in a probe molecule comprising a fluorescein moiety (label) covalently bound to the mono- or disaccharide (substrate).

Other examples of linking groups, in addition to hydroxyl, include halide (such as chloride, bromide or iodide), amine, amide, azide, hydrazide, carboxyl, imides (such as carbodiimide, maleimide and succinimide), acetyl halide, thiol, nitrile groups, cyanate groups, isothiocyanate, organosilane groups and siloxane groups.

For label molecules comprising metals such as copper or barium, then the metal can be bound to the label molecule through one or more charged ligands, such as carboxylate, or through charged or uncharged ligand polydentate ligands, for example polyethers, polyamines, porphyrins, crown ethers and cryptands.

In another embodiment, the label is an isotopically enriched analogue of the substrate molecule itself, for example being enriched with one or more isotopes that are radioactive or NMR-active, typical examples being ¹³C, ¹⁴C, ²H or ³H enriched substrate molecules.

An advantage of the present method of detecting mycobacteria is that in vivo imaging techniques such as PET, MRI, SPECT and CT can be employed, which are non-invasive, and can provide information not only on the presence of mycobacteria, but also where mycobacteria are concentrated within an organism's body, and additionally the metabolic state of the bacteria. The present invention also provides advantages for in vitro analysis of biological samples taken from an organism, in that the technique is relatively quick and facile, does not involve too many or too complicated processing steps, and has a smaller chance of false positives due to the specificity of the technique towards mycobacteria.

In a further embodiment of the invention, the probe molecule can comprise a functional group which can react with an amino acid residue in the enzyme, to form a chemical bond thereto. Such a functional group is henceforth referred to as a reactive group, and is preferably either a good leaving group, typically an electrophilic leaving group such as tosylate. In another embodiment, the reactive group can be an epoxide, phosphoryl fluoride, alpha-bromo, chloroester or a reactive oxoanion, such as a sulphonate, phosphate or phosphonate, which reacts with functional groups such as —OH on an amino acid residue of the enzyme to form an etheric bridge between the probe molecule and the enzyme, for example with serine residues. In a preferred embodiment, the reactive group of the probe molecule is able to react with a reactive amino acid residue present in the active site of the Ag85A, B and C enzyme molecule, preferably the Ser126 residue of Ag85B or the corresponding Ser residues in Ag85A and C, to form a chemical bond between the enzyme and the probe molecule. This provides greater specificity between the probe molecule and the Ag85 enzyme, which improves selectivity of the probe molecule towards mycobacteria.

In mycobacteria, the Ag85A, B and C enzymes use Tre, TMM and TDM as substrate molecules. The active sites of Ag85A, B and C are all structurally analogous, and hence a substrate molecule that is able to dock with the active site of one of Ag85A, B and C is capable of also docking with the corresponding active sites of the others. In relation to Ag85B, hydrogen bonding interactions between Tre, TMM or TDM and the active site occur at residues Arg 43, Trp 264, Ser126, His 262 and Leu 42, in which hydrogen bonds between OH groups of the Tre substrate and the relevant nitrogen or oxygen group on the amino acid can be formed. It has been found that, for a probe molecule to dock effectively into the active site, the ability to form a hydrogen bond simultaneously with at least two of these residues is important, at least one of which is preferably selected from with Ser126, and preferably at least one other with His 262. Due to the homologous nature of the active sites of Ag85A and C, then this also applies to corresponding interactions therein.

In one embodiment the probe molecule is based on a carbohydrate substrate, the carbohydrate having 1 to 6 monosaccharide units, optionally glycosidically linked, preferably 1 to 4 monosaccharide units. Carbohydrates are more preferably selected from monosaccharides and disaccharides. At least one of the monosaccharide units, typically a terminal monosaccharide unit of the carbohydrate, preferably comprises a six-membered ring. Most preferred carbohydrates are disaccharides, and even more preferred are disaccharides in which both monosaccharide units have 6-membered rings. This is generally because they are more structurally analogous to Tre, and hence are more likely to interact with more of the relevant residues in the enzyme active sites, and hence will generally provide improved selectivity for and uptake into mycobacteria.

Optionally, the carbohydrate can be derivatised. Herein, a derivative of a carbohydrate is a molecule which is based on a naturally occurring carbohydrate, with one or more of the hydroxyl or hydrogen atoms being replaced with other chemical moieties (herein derivative groups) which derivative groups do not substantially affect the ability of the probe molecule to engage with the active sites of the Ag85 A, B or C enzymes, and hence which do not prevent incorporation of the probe molecule into mycobacteria. Such derivative groups preferably do not inhibit the reactions which relate to incorporation of the probe molecule into the mycobacteria. In addition, such derivative groups are typically selected so as not to introduce instabilities into the probe molecule, for example by providing two anionic and/or nucleophilic groups on the same carbon atom, examples being two hydroxide groups, a hydroxide and ether group, or a hydroxide and halide group on the same carbon atom. Therefore, the carbohdyrates or derivatives thereof preferably have no more than one such derivative group per carbon atom of the carbohydrate substrate molecule. Examples of derivative groups, henceforth represented by X, include;

halides, R^{a} groups, —Z—H groups, groups of general formula —Z—R^{a}, groups of general formula WH₂₋ₓR^{a}ₓ, groups of general formula —C(Z)Z″-H, —C(Z)—R^{a}, —C(Z)Z″—R^{a} and —C(Z)—WH_{1-y}R^{a}_{y}; in which;
R^{a} is, at each occurrence, an optionally substituted linear or branched alkyl, alkenyl and alkynyl group or an optionally substituted aromatic group; R^{a} preferably comprises 6 carbon atoms or less; where R^{a} comprises one or more substituents, such substituents are selected from halides, —ZH, Z—R^{b}, WH₂₋ₓR^{b}ₓ, where R^{b} at each occurrence is selected from optionally substituted linear or branched alkyl, alkenyl, alkynyl and aromatic groups, preferably comprising 6 carbons atoms or less;
Z, Z′ and Z″ at each occurrence is a Group 16 element preferably selected from O and S, and preferably at least one of Z′ and Z″ is O;
W is a Group 15 element that is preferably selected from N and P;
x is 0, 1 or 2, and y is 0 or 1.

In one embodiment, the probe molecule is based on a carbohydrate represented by Formula I:

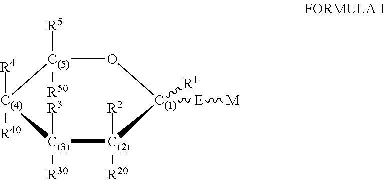

In this formula, M is a carbohydrate or derivative thereof comprising preferably 1 to 5 monosaccharide units, preferably 1 to 2 monosaccharide units. More preferably M is a monosaccharide unit or derivative thereof, most preferably a monosaccharide with a 6-membered ring. M is linked to C₍₁₎ through bridge group E, in either α or β configurations;

R¹ is selected from H, -L, —X, —CYY′L, —CYY′X;

One of R² and R²⁰ is —H and the other is selected from —OH, -L, —X, —CYY′L, —CYY′X;

One of R³ and R³⁰ is —H and the other is selected from —OH, -L, —X, —CYY′L, —CYY′X;

One of R⁴ and R⁴⁰ is —H and the other is selected from —OH, -L, —X, —CYY′L, —CYY′X; preferably R⁴ is —H and/or R⁴⁰ comprises a group able to form hydrogen bonds, for example —OH or —SH, which improves the extent of hydrogen bonding interaction at the Gly41 residue;

One of R⁵, and R⁵⁰ are —H, and the other is selected from CYY′L or —CYY′X; —

X is optional and is as defined above; Y and Y′ are independently H or X, with the proviso that the carbon atom to which they are bound has no more than one directly bound O, Z and W atoms; and wherein there is at least one label group L on the substrate molecule and/or at least one carbon atom in the molecule is ¹³C or ¹⁴C enriched, and/or at least one hydrogen atom in the molecule is ²H or ³H enriched.

E can be one or more of (a) a Group 16 element, preferably O, S or Se, or E; (b) a group comprising a Group 15 element with formula WH_{(1-(y+y′))}R^{a}_{y}L_{y′} in which W is preferably N or P, y and y′ are independently 0 or 1, and y+y′ is no more than 1; (c) a group comprising a Group 14 element of general formula VX′_{(2-(x+x′)}R^{a}ₓL_{x′} in which V is the Group 14 element, preferably C or Si, X′ is at each occurrence H, OH or X, x and x′ are individually 0, 1 or 2, x+x′ being no more than 2; the bridge E between carbon C₍₁₎ and M can optionally comprise more than one of (a) to (c) linked together, the bridge preferably comprising 4 or fewer bridging atoms, i.e. 4 or less (a) to (c) groups, preferably comprising 1 or 2 bridging atoms.

In another embodiment, the probe molecule is represented by Formula II;

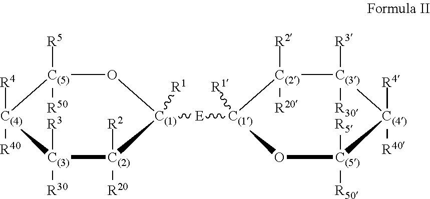

Where;

E is a bridging group as defined above, and each of the two monosaccharides are either α or β linked. Preferably at least one of the carbohydrates is a linked.

R¹ and R¹′ are independently selected from H, -L, —X, —CYY′L, —CYY′X;

One of R² and R²⁰ is —H and the other is selected from —OH, -L, —X, —CYY′L, —CYY′X;

One of R²′ and R²⁰′ is —H and the other is selected from —OH, -L, —X, —CYY′L, —CYY′X;

One of R³ and R³⁰ is —H and the other is selected from —OH, -L, —X, —CYY′L, —CYY′X;

One of R³′ and R³⁰′ is —H and the other is selected from —OH, -L, —X, —CYY′L, —CYY′X;

One of R⁴ and R⁴⁰ is —H and the other is selected from —OH, -L, —X, —CYY′L, —CYY′X;

One of R⁴′ and R⁴⁰′ is —H and the other is selected from —OH, -L, —X, —CYY′L, —CYY′X;

One of R⁵ and R⁵⁰ is —H, and the other is selected from CYY′L or —CYY′X;—

One of R⁵′ and R⁵⁰′ is —H, and the other is selected from CYY′L or —CYY′X;—

in which;

at least one of R⁴ and R⁴′ is H and/or at least one or R⁴⁰ and R⁴⁰′ is able to form hydrogen bonds, for example —OH or —SH, which enables either R⁴⁰ or R⁴⁰′ to effectively form a hydrogen bond with the Gly41 residue;

X is optional and is as defined above;

Y and Y′ are independently H or X, with the proviso that the carbon atom to which they are bound has no more than one directly bound O, Z and W atoms; and

wherein there is either at least one label group L on the molecule and/or one or more carbon atoms in the molecule is ¹³C or ¹⁴C enriched and/or at least one hydrogen atom in the molecule is ²H or ³H enriched. Preferably, R¹ and R¹′ are independently —H, or alkyl with less than 6 carbon atoms such as methyl; R², R²′, R²⁰, R²⁰′, R³, R³′, R³⁰, R³⁰′, R⁴, R⁴′, R⁴⁰ and R⁴⁰′ are each independently —H, —OH or -L; and R⁵ and R⁵′ are each independently either CH₂OH or CH₂L.

In a preferred embodiment R¹ and R¹′ are each H or methyl; R², R³⁰, R⁴, R⁵⁰, R²′, R³⁰′, R⁴′ and R⁵⁰′ are each independently H or L; R²⁰ and R²⁰′ are each independently H, L or OH; R³, R⁴⁰, R³′ and R⁴⁰′ are each independently —OH or L; and R⁵ and R⁵′ are each independently CH₂OH or CH₂L. In a further embodiment, either R⁴ is —OH or L and R⁴⁰ is H or L, or alternatively R⁴′ is —OH or -L and R⁴⁰′ is H or L, at least one of R⁴ or R⁴′ being H.

In preferred embodiments, where derivative groups, X, are present, they typically have an effective diameter of less than 1 nm, and more preferably less than 0.5 nm, to prevent excessive interference to the binding of the probe molecule with the enzyme active site.

The label, L can comprise an NMR-active nucleus, for example ¹⁹F, or ¹³C or ²H at higher than natural abundancies, a radioactive nucleus such as ¹⁴C or ³H at higher than natural abundancies, including positron emitting nuclei such as ¹⁸F or ¹²⁴I at higher than natural abundancies, or a heavy X-ray absorbing element such as iodine.

In one embodiment, L is selected from groups defined by X above, and which are isotopically enriched in one or more radioactive, NMR-active and/or positron emitting nuclei, for example isotopically enriched with one or more of, ¹³C, ¹⁴C, ²H, ³H, ¹⁸F, or ¹²⁴I.

In another embodiment, L is selected from groups defined by X above, which have at least one nucleus that can be detected using magnetic resonance imaging techniques at naturally occurring abundancies, such as F (which has a natural ¹⁹F abundance of 100%) and other X groups comprising F, for example a fluorobenzyl group (—CH₂—C₆H₄F).

In a further embodiment, L is selected from groups defined by X above, which have at least one nucleus that is large/heavy enough for detection using X-ray photographic or CT techniques, for example iodine and other X groups comprising iodine.

The label, L, can comprise a fluorophor, for example fluorophors selected from fluoresceins, xanthenes, cyanines, naphthalenes, coumarins, oxadiazoles, pyrenes, oxazines, acridines, arylmethines, Alexa Fluors, tetrapyrroles and quantum dots, the label L being the relevant portion of the label molecule that is attached to the substrate molecule after reaction therewith to produce the probe molecule.

Where L is a label such as a fluorophor that derives from a separate label molecule bound to the substrate molecule, then it can be represented by a general formula L′L″, where L′ is a connecting group and L″ comprises the detectable label.

The connecting group L′ can be selected from one or more of;

—Z—, —WH_{(1-y)}, R^{a}_{y}—, —C(Z)—WH_{(1-y)}, R^{a}_{y}—, C(Z′)Z″—, C(Z′)Z″—WH_{(1-y)}, R^{a}_{y}—C(Z′)Z″— (such as carbodiimide, maleimide and succinimide), —CH_{(1-y)}R^{a}_{y}═N—, —ZCH_{(1-y)}R^{a}_{y}N—, —NCH_{(1-y)}R^{a}_{y}Z—, NH_{(1-y)}R^{a}_{(y)}ZCN where Z, W, R^{a} and y are as defined above. The L″ group that comprises the label comprises in one embodiment a fluorophor, for example fluorophors selected from fluoresceins, xanthenes, cyanines, naphthalenes, coumarins, oxadiazoles, pyrenes, oxazines, acridines, arylmethines, Alexa Fluors, tetrapyrroles and quantum dots, and which are chemically attached to L′.

An example of an L′ and L″ combination is L′=NH and L″ is fluorescein isothiocyanate as shown in the molecular structure below (formula A);

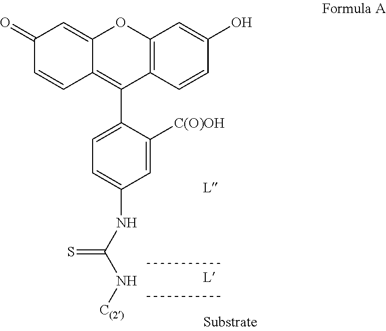

In this specific example, in which the probe molecule is represented by Formula II, the L group is attached to carbon C_{(2′)} of the substrate, the L′ (connecting) portion of the L group is NH, and the L″ label-containing portion of the L group comprises a fluorescein. This particular combination is formed from the reaction of fluorescein isothiocyanate (FITC) with an amine-modified carbohydrate. One with skill in the art will understand from this illustration that other combinations of connecting groups and label-containing groups are possible for other labels, for example L″ groups comprising other fluoresceins or other fluorophores, and L′ groups comprising different connecting groups.

Where the probe molecule comprises a reactive group, which can react with an amino acid residue on the enzyme to covalently link the probe molecule and enzyme, the reactive group can optionally comprise the label, in which the label portion of the reactive group remains bound to the substrate after reaction with the enzyme. The reactive group is preferably chosen and positioned on the probe molecule such that it can chemically react with one of the active site enzymes selected from Gly41, Ser126, Asn223, Arg43 and Trp264 in the Ag85B enzyme active site, and corresponding residues in Ag85A and C. In a preferred embodiment, the reactive group is placed on or is attached to group R⁵ (formula I) or either R⁵ or R^{5′} (Formula II). Thus, in one embodiment of the invention, the probe molecule comprises a reactive group, G, which can optionally comprise a label, where, in the case of compounds of Formula I or Formula II, the group G can replace an X group. G is preferably selected from groups comprising one or more of the following; a phosphate, phosphonate, phosphoryl fluoride, an organophosphate, an epoxide, a tosylate, a sulphonate alpha bromo or chloro ester.

Trehalose and derivatives thereof are preferred choices as the substrate of the probe molecule, being based on the natural substrate for the Ag85 A, B and C enzymes. Monosaccharide compounds, such as labelled glucose or arabinose, or derivatives thereof, can be used as probe molecules because they can be incorporated into a mycobacterial cell wall through the action of Ag85 enzymes, although the extent to which they are incorporated is relatively poor compared to disaccharides such as Tre or derivatives thereof. Additionally, such monosaccharides are generally less selective towards mycobacteria, and hence could provide greater chance of providing false positives when testing for mycobateria.

Ag85A, Ag85B, and Ag85C share high sequence and structural homology (Ronning, D. R. et al; Nat Struct Biol 7 (2), 141 (2000); Anderson, D. H. et al; J Mol Biol 307 (2), 671 (2001); Ronning, D. R. et al; J Biol Chem 279 (35), 36771 (2004)), characterized by an α,β-hydrolase fold and a hydrophobic fibronectin-binding domain. Their active sites are highly conserved, and features a hydrophobic tunnel for the mycolic acid.

The inventors have found from a structural analysis that the C₍₂₎ carbon of compounds of Formula I and the C₍₂₎ and C_{(4′)}, or C_{(2′)} and C₍₄₎, carbons of compounds of Formula II tend to point outwards away from the Ag85 A, B or C enzyme when bound thereto. This means that the enzyme can tolerate bulky derivative or label groups on a probe molecule when bound preferably at these sites, such bulky groups being able to have diameters of greater than 10 nm, or greater than 20 nm, and even of the order of 100's of nanometers (for example in the case of quantum dots) without affecting the ability of the probe molecule to act as a substrate for the catalysed transesterification reaction. Therefore, although enzymes often require high substrate specificity in order to act as catalysts, it has been found that the Ag85 enzymes in mycobacteria can tolerate a lack of specificity, in particular at certain positions on a substrate molecule, such that they do not preclude the required binding at the active enzyme site. In addition, because of the position of the hydrophobic tunnel into which mycolate groups bind during the transesterification reactions, then preferably the groups R⁵ and R⁵′ are CYY′OH, in which Y and Y′ are preferably hydrogen.

In a typical method for determining whether mycobacteria are present, the probe molecule is added to a biological sample taken from an organism, for example a sample of sputum, cerebrospinal fluid, pericardial fluid, synovial fluid, ascitic fluid, blood, bone marrow, urine or faeces. Alternatively, the probe molecule can be administered to the organism directly, for example an animal, in particular mammals including humans, wherein detection of mycobacteria can be carried out through imaging techniques, or by taking of a sample from the organism and testing the removed sample ex vivo for the presence of the mycobacteria-incorporated label.

An advantage of the present invention is that the test is not necessarily specific to individual mycobacteria species, and can be used to detect a number of different species or types that can affect different organisms. Additionally, the probe molecules once incorporated into the mycobacteria, are able to pass into infected cells, such as macrophages, without being damaged. A significant feature of the present invention is that, for human infections, Mtb can be labelled with the probe molecule, either in vivo or in vitro on a biological sample, enabling efficient detection of this damaging disease-causing bacterium. The use of the probe molecule comprising a label according to the present invention, particularly its use in detection in vivo through imaging techniques, also offers scope for understanding the progress of a mycobacterial disease, for example to track bacterial transit to the phagosome and other intracellular compartments.

An additional advantage of the present invention is the possibility of diagnosing M. Avium, which is an opportunistic infection often acquired by HIV-positive patients.

In the preparation of probe molecules, label groups can be attached directly to a substrate molecule to form the probe molecule. Alternatively, a precursor to the substrate can first be labelled, which precursor can then be reacted with one or more other substrate precursors to produce the probe molecule. For example, where the substrate is a monosaccharide or disaccharide, the monosaccharide or disaccharide can be reacted with the label to produce the chemically bound label. Alternatively, for example where the substrate to be labelled is a disaccharide, a labelled monosaccharide can be prepared (labelled substrate precursor) which, after reaction with another monosaccharide (another substrate precursor) to form a glycosidic link, to form the labelled disaccharide (probe molecule). In a further embodiment, as explained above, the substrate molecule or precursor thereof can be prepared with a functional group to which the label can be chemically attached.

To prepare labelled molecules of Tre or a derivative thereof such as methyl-trehalose (Me-Tre), the inventors have used two approaches. One approach, outlined in Scheme I below (a), involves taking two monosaccharide precursors, one labelled and/or derivatised, and both with protected OH groups at all positions except for the carbons in the 1 position (equivalent to C₍₁₎ and C_{(1′)} in Formula II above), and forming a glycosidic bond between the two monosaccharide units. The protecting groups are then removed to produce the labelled and or derivatised disaccharide probe molecule. Glycosidic bonds can be formed using enzyme-catalysed reactions, or through non-enzymatic reactions. Known reactions include through ketoside formation (Yamanoi, Takashi et al., Tetrahedron: Asymmetry 17 (20), 2914 (2006); Namme, et al; European Journal of Organic Chemistry 2007 (22), 3758 (2007); Gomez, Ana M. et al; Tetrahedron Letters 44 (32), 6111 (2003); Griffin, Frank K et al; (2002), Vol. 2002, pp. 1305; Yang, W. B. et al., Stereochemistry in the synthesis and reaction of exo-glycals. J Org Chem 67 (11), 3773 (2002); Tiwari, P. and Misra, A. K., (2006), Vol. 71, pp. 2911; Zhu, X., Jin, Y., and Wickham, J., (2007), Vol. 72, pp. 2670), dehydrative glycosylation (Rodriguez, Miguel Angel et al., The Journal of Organic Chemistry 72 (23), 8998 (2007)), and chemoenzymatically (Giaever, H. M. et al; J Bacteriol 170 (6), 2841 (1988); Gibson, R. P. et al., J Biol Chem 279 (3), 1950 (2004)). For derivatised molecules, the label group, for example a benzyl fluoride or fluorescein group, is subsequently attached by reaction with the label molecule to form the final probe molecule.

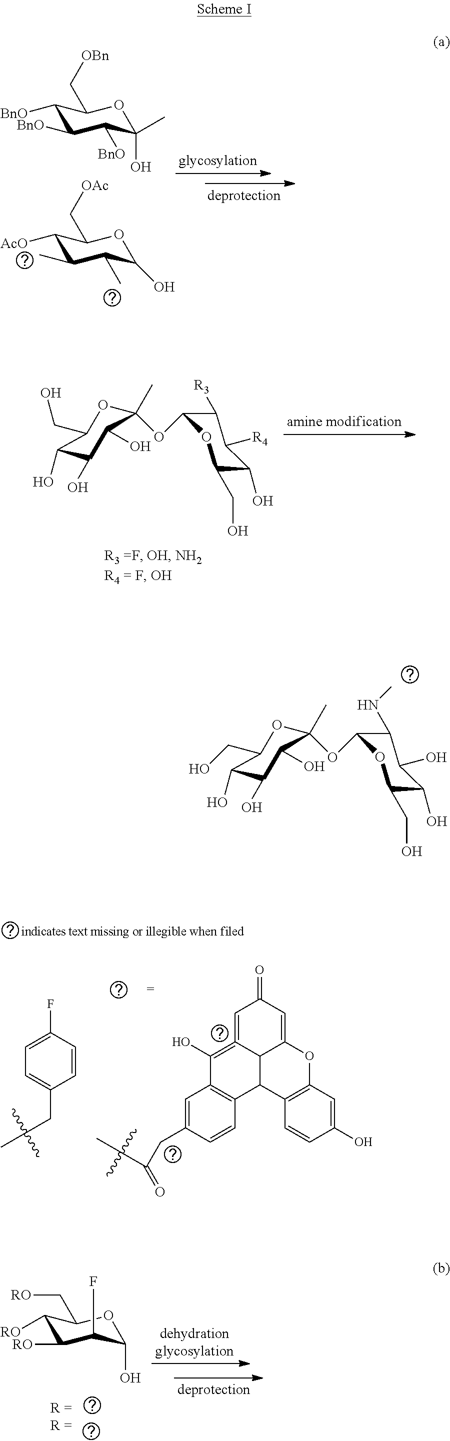

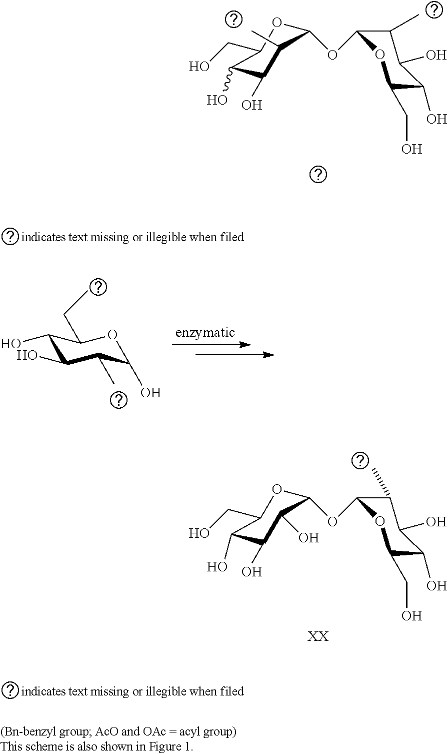

In another approach, also illustrated in Scheme I above (b), two labelled Tre monomer precursors are combined in a dehydration reaction to form a glycosidic bond, wherein the two monosaccharides are symmetrically labelled at the same position (corresponding to the C₍₂₎ and C_{(2′)} carbons of Formula II above).

In a third approach, also in scheme I (c) above, a fluorine-labelled and phosphate-derivatised Tre precursor monosaccharide is combined with a non-functionalised or labelled Tre monomer precursor in an enzyme-catalysed reaction to produce a labelled Tre disaccharide probe molecule.

In a further approach, outlined in scheme II below, trehalose itself can be labelled, or modified with functional groups that can react further with a label-containing molecule to produce labelled Tre.

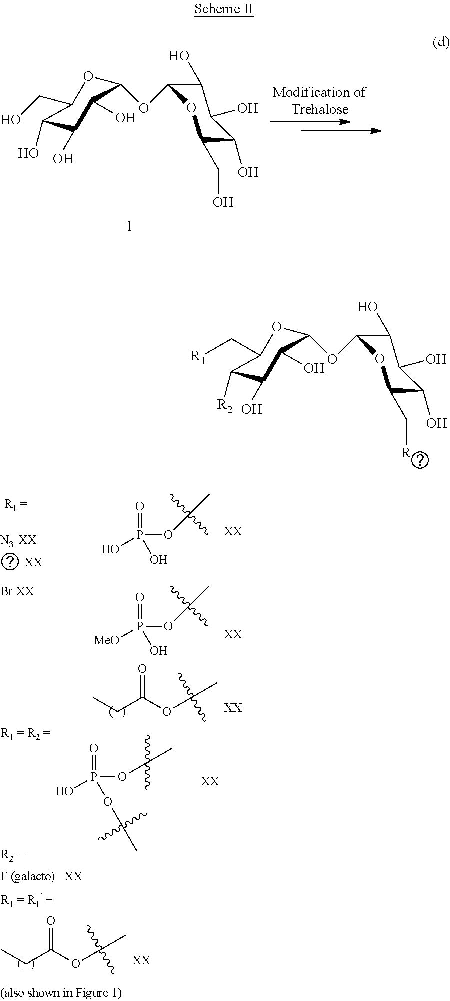

The invention will now be illustrated by the following non-limiting examples, with reference to the Figures in which;

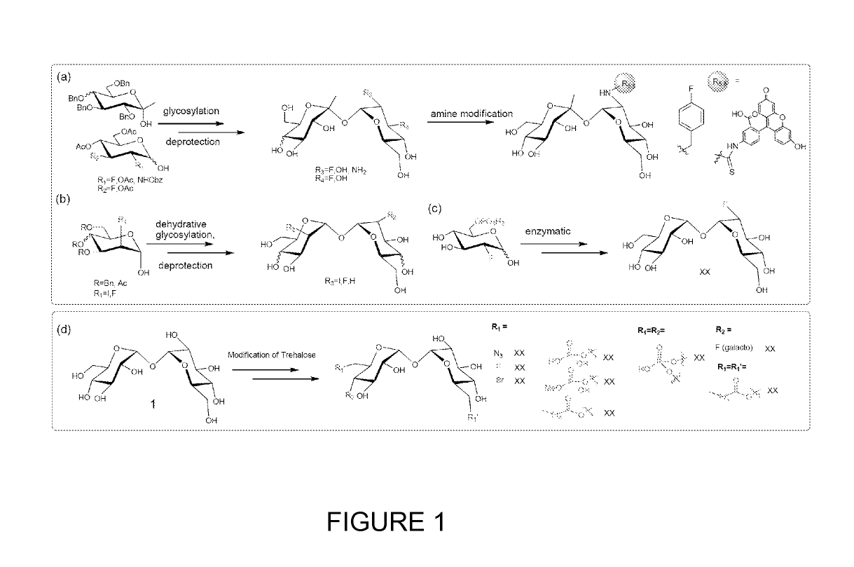

FIG. 1 shows Schemes I and II above;

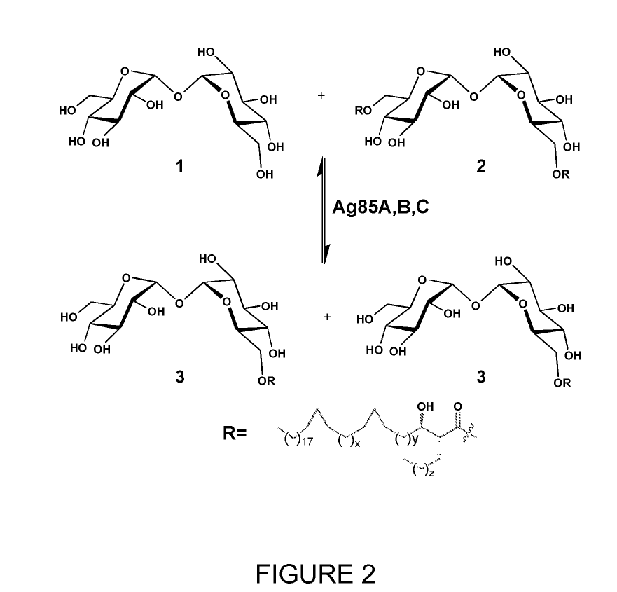

FIG. 2 illustrates the transesterification reaction of TDM and Tre to produce TMM, catalysed by Ag85 enzymes;

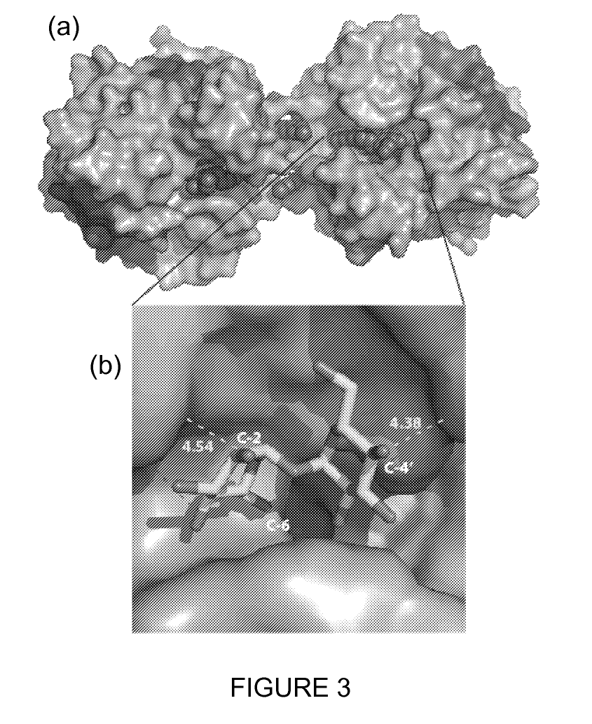

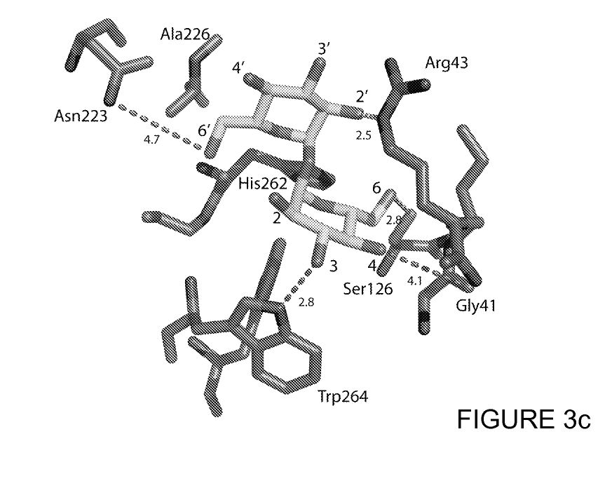

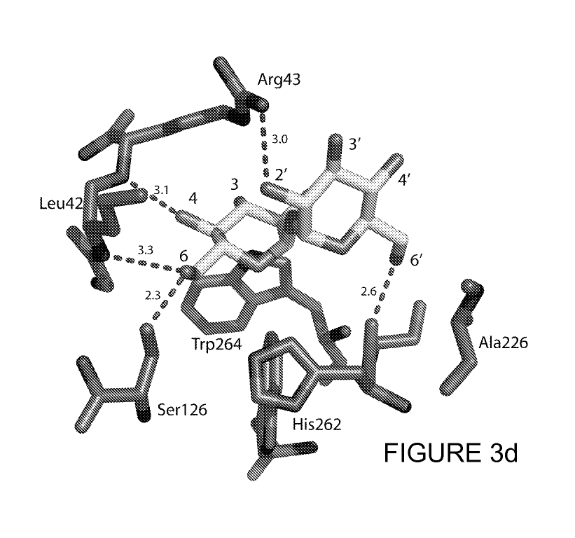

FIG. 3(a) illustrates the binding of octylthioglucoside in Ag85C dimer FIG. 3(b-d) illustrate the binding of a trehalose substrate molecule in the active site of Ag85B;

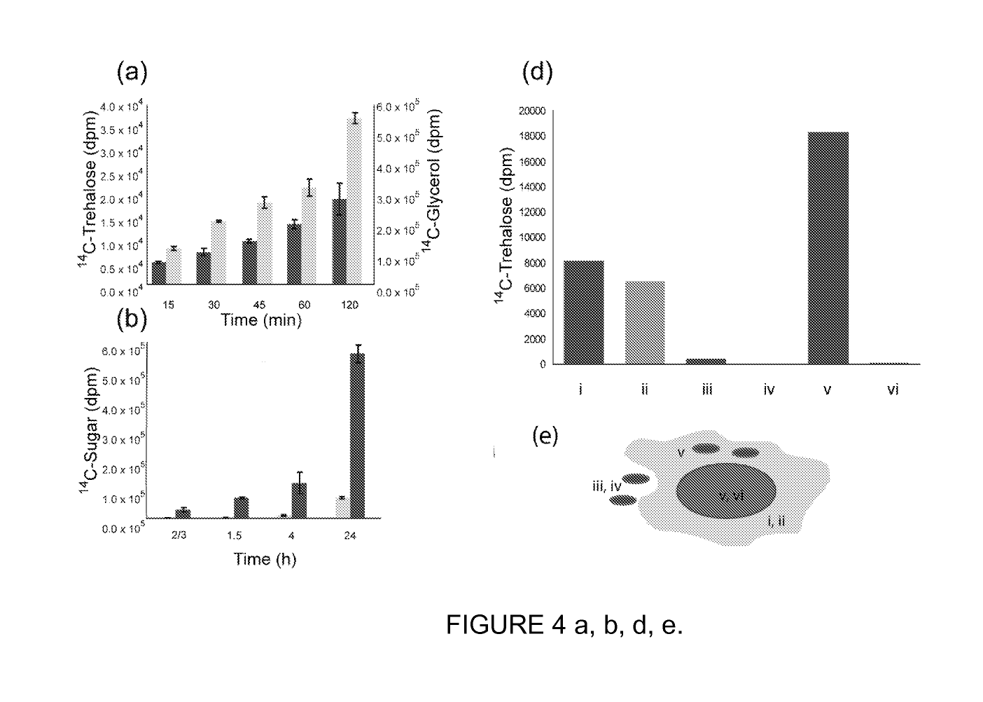

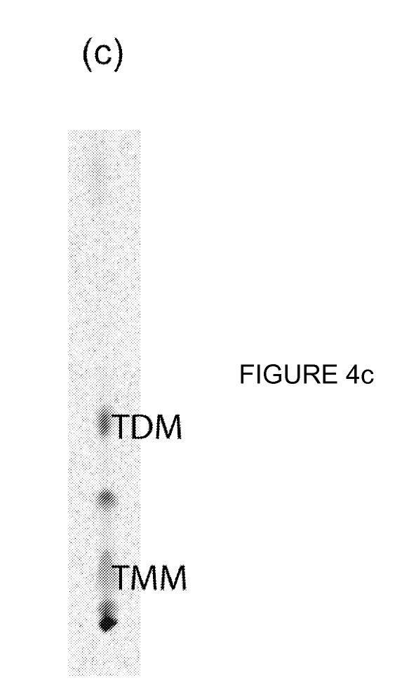

FIG. 4(a) is a graph showing uptake of ¹⁴C labelled Tre in an Mtb culture over time compared to ¹⁴C labelled glycerol;

FIG. 4(b) is a graph showing uptake of ¹⁴C labelled Tre in an Mtb culture over time compared to ¹⁴C labelled glucose;

FIG. 4(c) is a radiographic TLC (thin layer chromatography) of a lipid extract from Mtb, in which ¹⁴C-labelled Tre has been incorporated.

FIG. 4(d) is a graph showing uptake of ¹⁴C-Tre into Mtb-infected macrophages;

FIG. 4(e) illustrates the different parts of the macrophages analysed for ¹⁴C-Tre uptake;

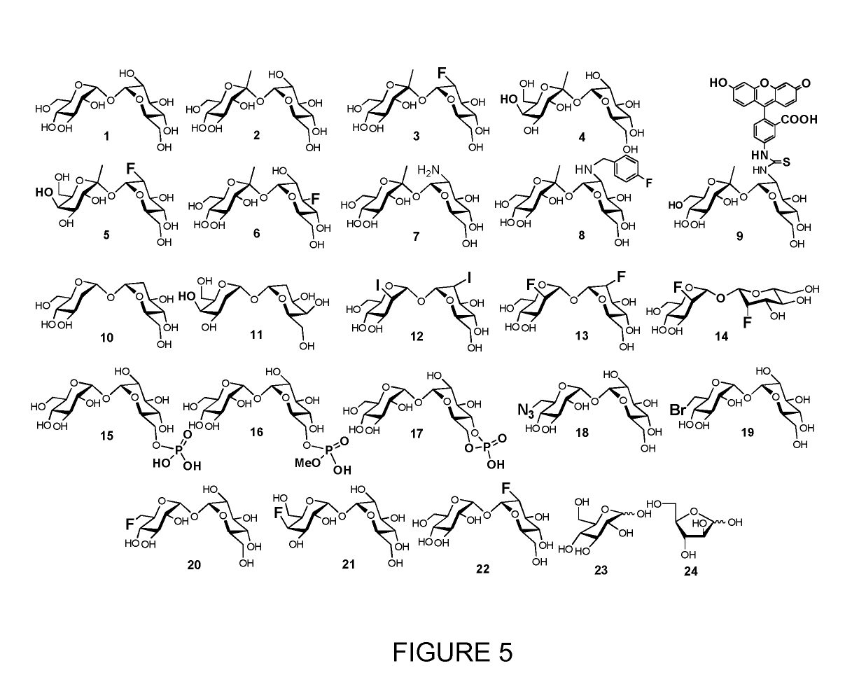

FIG. 5 shows different molecules that were tested for uptake into Mtb;

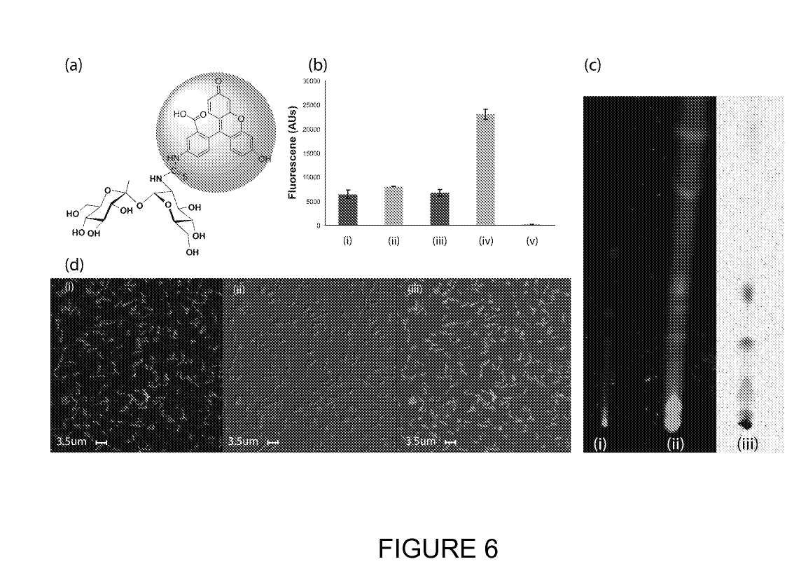

FIG. 6(a) shows the structure of a FITC-labelled Tre substrate;

FIG. 6(b) is a graph showing uptake of FITC-labelled Tre into live and heat-killed Mtb, as determined by fluorescence;

FIG. 6(c) shows TLC plates highlighting incorporation of FITC-labelled Tre into glycolipids extracted from MtB;

FIG. 6(d) is a series of images showing fluorescence characteristics of Mtb with incorporated FITC-labelled Tre.

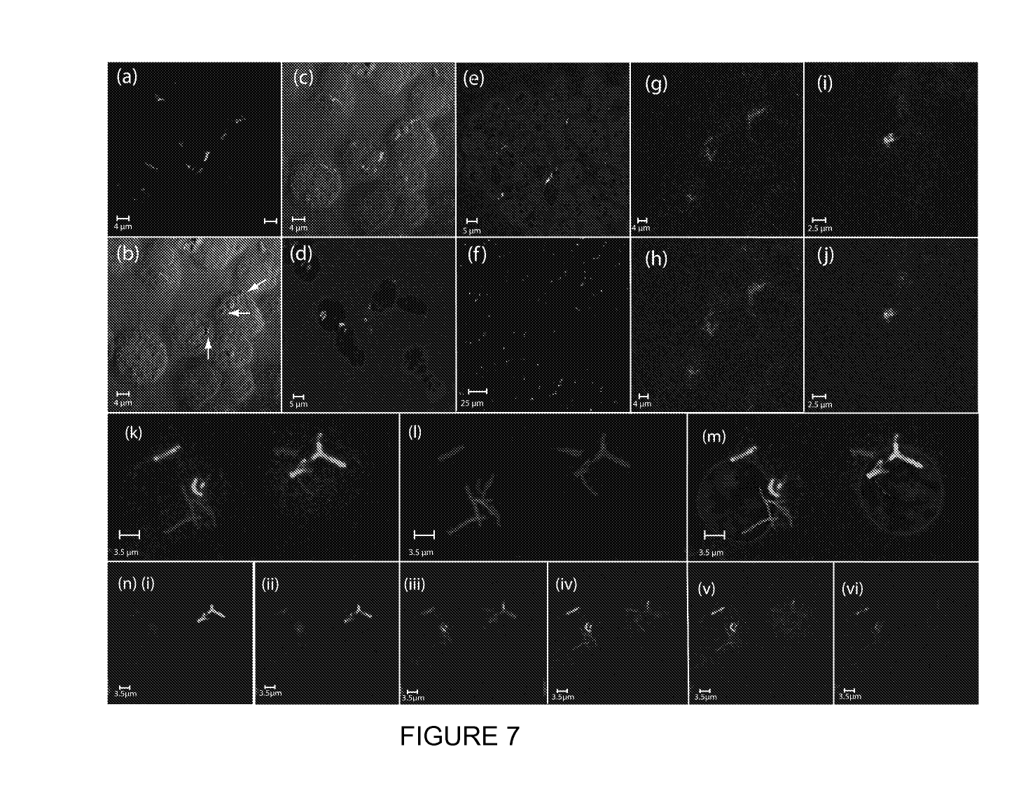

FIG. 7 is a series of images of macrophages infected with RFP BCG vaccine or Mtb comprising FITC-labelled Tre.

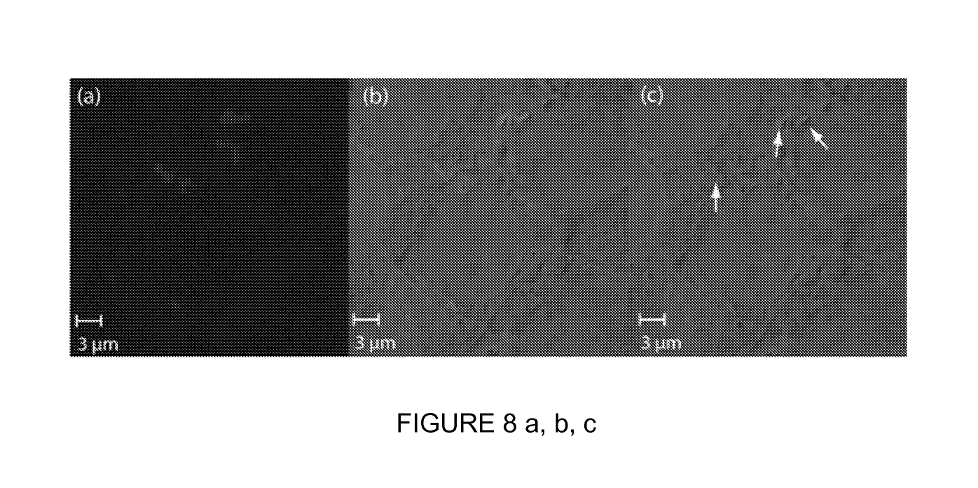

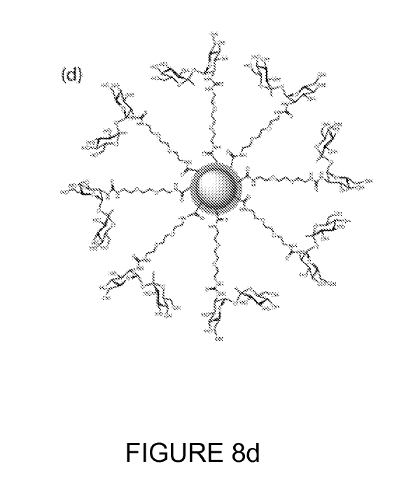

FIG. 8 illustrates the synthesis of a quantum dot (QD)-labelled Tre substrate (S.I.).

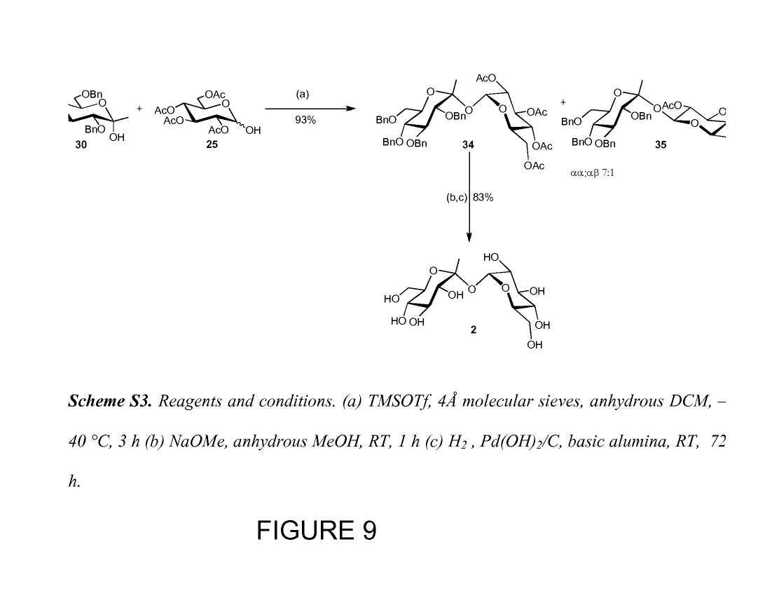

FIG. 9 illustrates a representative synthesis of methyl-derivatised FITC-Tre (S.I.).

In FIG. 1, schemes illustrating synthetic routes to providing various labelled carbohydrates, in particular labelled disaccharides are shown. They are described in further detail above. (a) represents synthesis of methyl-Tre analogues, (b) dehydrative glycosylations to produce symmetric difluoro, diiodo and dideoxy Tre; (c) 2-fluoro_Tre enzymatic synthesis with Tre-6-phosphate synthase; (d) 4- and 6-fluoro Trehalose; (e) phosphoryl Tre derivatives, and (f) Hexanoyl and dihexanoyl Tre.

FIG. 2 shows one of the transesterification reactions catalysed by Ag85 enzymes, in this case the transesterification of Tre, 1, and TDM, 2, to produce two molecules of TMM, 3. The R group represents the mycolate group, where x is typically 10-20, y is typically 17-20, and z is typically 25-30.

FIG. 3(a) shows a dimeric molecule of Ag85C, and indicates where a substrate carbohydrate molecule binds, in this case octyl thioglucoside.

FIG. 3(b) illustrates how a Tre molecule interacts with an Ag85B enzyme. From this view, it can be seen that the substrate carbon atoms, C₍₂₎ and C_{(4′)} point away from the enzyme active site, which enables large label groups to be attached thereto without substantial interference with the docking of the remainder of the molecule with the active site. Also shown in this diagram is a hydrophobic channel or tunnel in the enzyme, into which the mycloate group bound to carbon atom C₍₆₎ points. FIGS. 3(c) and 3(d) are alternative views of 3(b), highlighting amino acid residues associated with the Ag85B active site, and bonding to the Tre-substrate.

FIG. 4 demonstrates how labelled carbohydrate substrates are incorporated into mycobacteria, specifically Mtb. FIG. 4(a) compares the uptake of ¹⁴C-labelled Tre (left) with ¹⁴C-labelled glycerol (right) over time, glycerol being used as a positive control because it is known to be taken up into mycobacteria. FIG. 4(b) similarly compares uptake of two labelled carbohydrate substrates, namely ¹⁴C-Tre (right) and ¹⁴C-glucose (left), over time showing the more efficient uptake of Tre compared to glucose. FIG. 4(c) shows a radiographic TLC plate of a lipid extract from Mtb, showing the presence of labelled TDM (*) and TMM (**) resulting from incorporation of the ¹⁴C-Tre. The solution used was 4:1 chloroform:methanol. FIG. 4(d) illustrates uptake of ¹⁴C-labelled Tre into tuberculosis-infected and control macrophages. With additional reference to FIG. 4(e), which shows a scheme of the different cellular compartments evaluated, (i) represents the cytoplasm of infected macrophages, (ii) cytoplasm of control macrophages (i.e. uninfected), (iii) floating Mtb from infected macrophages, (iv) floating debris from control macrophages, (v) Mtb from infected macrophages, and (vi) an extracted pellet from control macrophages.

FIG. 5 illustrates the molecules tested for uptake into Mtb. Molecules labelled 1 to 22 are all based on Trehalose. Molecule 23 is based on glucose, and 24 is based on arabinose. Molecules 5 and 11 are in the galacto-form.

FIG. 6(a) shows the molecular structure of FITC-Tre probe molecule, i.e. Tre substrate labelled with an FITC molecule. FIG. 6(b) graphically illustrates the uptake of FITC-Tre into live [(ii) and (iv)] versus heat-killed [(i) and (iii)] Mtb over time. (i) and (ii) are after two hours, (iii) and (iv) are after 24 hours. (v) represents auto-fluorescence of untreated Mtb. FIG. 6(c) are TLC plates, in which (i) is a plate of FITC-Tre and (ii) is a plate of a lipid extract from FITC-Tre treated Mtb after fluorescence excitation at 486 nm. (iii) is a radio-TLC of a lipid extract from ¹⁴C-Tre treated Mtb. Images (ii) and (iii) are of the same plate, which was co-spotted with the ¹⁴C-Tre labelled Mtb extract FITC-Tre labelled Mtb extract, and show that differently labelled Tre probe molecules are incorporated into mycobacteria in a similar way. FIG. 6(d) shows images of FITC-Tre labelled Mtb, in which (i) is a fluorescence image showing the presence of a fluorescein, (ii) is a transmitted light differential interference contrast (DIC) image, and (iii) is an overlay of the fluorescence and DIC images, highlighting the correspondence between the Mtb bacteria and the presence of the FITC-Tre probe molecule.

FIG. 7 shows images of macrophages infected with RFP BCG vaccine (Red Fluorescent Protein-labelled Bacillus Calmette-Guerin vaccine) or Mtb. (a) Fluorescence image of Mtb labelled with FITC-Tre (green); (b) DIC image showing the macrophages with Mtb bacteria indicated by white arrows; (c) is an overlay of the DIC and fluorescence images, showing correspondence between the bacteria and the FITC-Tre label; (d) is a fluorescence image of FITC-Tre labelled Mtb-infected macrophages, in which the DNA of the macrophages has been stained blue using DAPI (4′,6-diamidino-2-phenylindole); (e) labelling at a lower concentration of 1 μM FITC-Tre, (gain on microscope increased); (f) zoomed out image of labelled infected macrophages; (g) image taken within 1 hour of adding the probe molecule; (h) overlay of 1 hour label with RFP; (i) FITC-Tre Mtb (green) treated with anti-Mtb antibody; (j) Overlay of FITC-Tre and antibody signal (antibody labelled with Alexa-594 fluorescent dye) shows colocalization of FITC-Tre and antibody; (k) FITC-Tre maximum projection through the cell; (1) RFP BCG maximum projection through cell (red); (m) overlay of maximum projections; (n) (i-vi) stack through cell (1 μm between slices).

In FIG. 8(a) to (c) are shown, fluorescence (a), DIC (c) and overlay (b) images of Mtb-infected macrophages, where Mtb is modified with QD-Tre probe molecule. The QD excitation was at 562 nm, with emission at 585 nm-634 nm. Arrows identify Mtb in the DIC plot as highlighted by the fluorescent emission.

In FIG. 8(d), the synthesis of a QD (quantum dot)-labelled Tre is shown. The QD label is bound to multiple Tre molecules, and hence in one embodiment of the invention a label can be shared with a plurality of substrate molecules. In this specific QD-label embodiment the QD comprised up to 110 Tre molecules.

### EXPERIMENTAL

Images of stained cells were obtained by confocal microscopy (Leica SP2, Leica Microsystems, Exton, Pa.) using a 63× oil immersion objective NA 1.4. All data were processed with Leica LAS software and Adobe Illustrator to compile images.

Protein concentrations were calculated using standard BCA assay or with a Labtek ND-1000 Nanodrop.

High Performance Liquid Chromatography was conducted on a Dionex UltiMate 3000 HPLC system at ambient temperature, with an in line variable UV absorbance detector, or a Varian PLS400 Evaporative Light Scattering detector (ELSD) parallel to the main flow path.

Hydrogenations were either performed manually or with a Thales Nano H Cube®.

Protein purification was performed on an AKTA Prime FPLC system (GE Healtchare).

Fluorescesce and radioactivity of lipid extractions was read on a Phosphorimager: Typhoon 9410 Variable Mode Imager by GE Healthcare Bio-Sciences.

Scintillation counting was conducted on an LS 6500 Multi-purpose scintillation counter by Beckman Coulter.

Fluorescence readings of Mtb were conducted on a FLUOstar Optima by BMG Labtech.

Images of stained cells were obtained by confocal microscopy (Leica SP2, Leica Microsystems, Exton, Pa.) using a 63× oil immersion objective NA 1.4. All data were processed by Leica LAS AF Lite and Adobe Illustrator Software.

Optical rotations were measured on a Perkin-Elmer 241 polarimeter with a path length of 1.0 dm and are reported with implied units of 10⁻¹ deg cm² g⁻¹. Concentrations (c) are given in g/100 mL.

Melting points (m.p.) were recorded on a Leica Galen III hot stage microscope eqpped with a Testo 720 thermocouple probe and are uncorrected.

Proton nuclear magnetic resonance (¹H NMR) spectra were recorded on a Bruker DPX400 (400 MHz), a Bruker AV400 (400 MHz) or a Bruker AVII500 (500 MHz) spectrometer, as indicated.

Carbon nuclear magnetic resonance (¹³C NMR) spectra were recorded on a Bruker AV400 (100 MHz) spectrometer or on a Bruker AVII500 (125 MHz) spectrometer, as indicated. NMR Spectra were fully assigned using COSY, HSQC, HMBC and DEPT 135. All chemical shifts are quoted on the δ scale in ppm using residual solvent as the internal standard (¹H NMR: CDCl₃=7.26, CD₃OD=4.87; DMSO-d6=2.50 and ¹³C NMR: CDCl3=77.0; CD3OD=49.0; DMSO-d6=39.5). Coupling constants (J) are reported in Hz with the following splitting abbreviations: s=singlet, d=doublet, t=triplet, q=quartet, quin=quintet, and a=apparent.

Infrared (IR) spectra were recorded on a Bruker Tensor 27 Fourier Transform spectrophotometer using thin films on NaCl plates for liquids and oils and KBr discs for solids and crystals. Absorption maxima (νₘₐₓ) are reported in wavenumbers (cm⁻¹) and classified as strong (s) or broad (br).

Low resolution mass spectra (LRMS) were recorded on a Waters Micromass LCT Premier TOF spectrometer using electrospray ionization (ESI) and high resolution mass spectra (HRMS) were recorded on a Bruker MicroTOF ESI mass spectrometer. Nominal and exact m/z values are reported in Daltons.

Thin layer chromatography (TLC) was carried out using Merck aluminium backed sheets coated with 60F254 silica gel. Visualization of the silica plates was achieved using a UV lamp (λmax=254 nm), and/or acid dip (1:1 MeOH/H₂O, 10% H₂SO₄) and/or ammonium molybdate 5% in 2M H₂SO₄, and/or potassium permanganate (5% KMnO₄ in 1M NaOH with 5% potassium carbonate). Column chromatography was carried out using BDH PROLAB® 40-63 mm silica gel (VWR). Mobile phases are reported in ratio of solvents (e.g. 4:1 petrol/ethyl acetate). Anhydrous solvents were purchased from Fluka or Acros with the exception of dichloromethane and THF, which were dried over Alumina cartiges. All other solvents were used as supplied (Analytical or HPLC grade), without prior purification. Distilled water was used for chemical reactions and Milli-Q™ purified water for protein manipulations.

Reagents were purchased from Sigma Aldrich and used as supplied, unless otherwise indicated.

Trehalose was purchased from Fluka. ‘Petrol’ refers to the fraction of light petroleum ether boiling in the range 40-60° C. All reactions using anhydrous conditions were performed using flame-dried apparatus under an atmosphere of argon or nitrogen. 3 Å and 4 Å molecular sieves were activated by heating in a 400° C. furnace and were also employed for anhydrous reactions.

Basic alumina refers to basic aluminum oxide and was utilized during some hydrogenation reactions.

Brine refers to a saturated solution of sodium chloride Anhydrous magnesium sulfate (MgSO₄) or sodium sulfate (Na₂SO₄) were used as drying agents after reaction workup, as indicated.

DOWEX 50WX8 (H+ form) was conditioned as follows: 100 g of the commercial resin was placed in a 500 mL sintered filter funnel and allowed to swell with 200 mL of acetone for 5 minutes. The solvent was removed by suction and the resin was washed successively with 800 mL of acetone, 500 mL methanol, 500 mL 5M HCl, and then 1 L of water or until the pH of filtrate was ˜7, as indicated by pH paper. The resin was partially dried on the filter and then stored and used as needed.

In addition to those specified above, the following abbreviations, designations, and formulas are used throughout:

Ar=Argon, MeOH=methanol, H₂O=water, Et₂O=diethyl ether, EtOH=ethanol, TFE=trifluoro ethanol, EtOAc=ethyl acetate, CH₂Cl₂=DCM=dichloromethane, DMF=dimethylformamide, iPrOH=isopropanol, Et₃N=triethylamine, K₂CO₃=potassium carbonate, NaHCO₃=sodium bicarbonate, NaOH=sodium hydroxide, NH₄Cl=ammonium chloride, NH₄OH=ammonium hydroxide, TFA=trifluoro acetic acid, aq.=aqueous sat.=saturated, TMSOTf=Trimethylsilyl trifluoromethanesulfonate, FITC=fluorescein isothiocyanate, Bn=benzyl, Ac=acetyl, TBDPS=tert-butyldiphenylsilyl, TBAF=tetra-n-butylammonium fluoride, DAST=diethylaminosulfur trifluoride, DMAP=4-Dimethylaminopyridine, PBS=phosphate buffered saline, TEA=triethanolamine, DAPI=4′,6-diamidino-2-phenylindole is a fluorescent stain that binds strongly to DNA, DIC=differential interference contrast image.

Protein Mass Spectrometry (LC-MS) was performed on a Micromass LCT (ESITOF-MS) coupled to a Waters Alliance 2790 HPLC using a Phenomenex Jupiter C4 column (250×4.6 mm-5 μm) Water:acetonitrile, 95:5 (solvent A) and acetonitrile (solvent B), each containing 0.1% formic acid, were used as the mobile phase at a flow rate of 1.0 mL min⁻¹.

The gradient was programmed as follows: 95% A (5 min isocratic) to 100% B after 15 min then isocratic for 5 min. The electrospray source was operated with a capillary voltage of 3.2 kV and a cone voltage of 25 V. Nitrogen was used as the nebulizer and desolvation gas at a total flow of 600 L h⁻¹. Spectra were calibrated using a calibration curve constructed from a minimum of 17 matched peaks from the multiply charged ion series of eqne myoglobin obtained at a cone voltage of 25V. Total mass spectra were reconstructed from the ion series using the MaxEnt algorithm preinstalled on MassLynx software (v. 4.0 from Waters) according to the manufacturer's instructions.

Selected syntheses of examples of compounds according to the present invention are now described. The indentities of those compounds referred to by number can be found further on in the description, under the heading “Schemes, Synthesis and Characterization”.

### Synthesis of Fluorescein-Labelled Trehalose (FITC-Tre), Compound 9

Compound 30 (1.75 g, 3.17 mmol, 1) and compound 27 (1.54 g, 3.5 mmol, 1.1 equi) were dried under reduced pressure for 1 hour and then dissolved in anhydrous CH₂Cl₂ (50 mL) and added to a dry flask in the presence of molecular sieves (ca. 1 g). The mixture was stirred with molecular sieves for 30 minutes at room temperature and then was cooled to −40° C. To this was added trimethylsilyl trifluoromethanesulfonate (TMSOTf) (100 μl, 0.54 mmol. 0.15 equi) at −40° C. under an Ar atmosphere. The resulting mixture was stirred for 1 hour until thin layer chromatography (TLC), using 1.5:1 petrol/ethyl acetate (by volume), revealed the production of two new products (Rf 0.6 and Rf 0.35) and incomplete consumption of starting material (Rf 0.35 and Rf 0.05). An additional portion of TMSOTf (100 μl, 0.54 mmol. 0.15 equi) was added to the reaction and reaction stirred for a further 1.5 hours, for a total 2.5 hours reaction time. The reaction was then quenched by the addition NEt₃ (0.1 ml), filtered through celite and concentrated. The crude product was purified by column chromatography (5:2 petrol/ethyl acetate by volume) to yield the desired compound 43 α,α (1.065 g) as well as the 44 α,β (212 mg) for a net yield (46%, 5:1α,α: α,β) as well as recovered compound 30 (184 mg, 10%) and recovered compound 27 (288 mg, 20%).

Compound 43 (578 mg, 0.59 mmol, 1 equi) was dissolved in anhydrous methanol (15 ml). To this was added sodium methoxide (25 mg, 0.46 mmol, 0.94 equi) and the reaction mixture was stirred for 1 hour, upon which time full conversion to product was detected by TLC (ethyl acetate) (Rf 0.75) and disappearance of starting sugar (Rf 1). The reaction mixture was neutralized with DOWEX 50WX8 (H⁺ form) cation exchange resin (ca 50 mg). The DOWEX was removed by filtration and the filtrate was concentrated under reduced pressure to yield the deacetylated product (507 mg, 100%). This product was split into two portions and used without further purification. Each portion was dissolved in 20 ml (1:1 trifluoroethanol/water) with formic acid (50 μl) and cycled through a ThalesNano H Cube® over a Pd/C cartridge for 10 hours. Upon completion product was detected by TLC (1:2:2 water/isopropanol/ethyl acetate volume) (Rf 0.05) with complete disappearance of starting sugar (Rf 1). The reaction mixtures were concentrated under reduced pressure, redissolved in water and lyophilized to yield the desired product compound 7 as a brownish, amorphous solid, which was further purified, utilizing a C18 sep-pak cartridge (221 mg, 93%).

Compound number 7 (11 mg, 0.03 mmol, 1 equi) and fluorescein isothiocyanate (17 mg, 0.044 mmol, 1.4 equi) were dissolved in 75 mM NaHCO₃ buffer at pH 9 (1 ml) with acetonitrile (0.5 ml). The mixture was heated to 50° C. for 2 hours upon which time Product (Rf 0.3) was detected by TLC (1:2:2 water/isopropanol/ethyl acetate—volume) with near complete disappearance of flourescein starting material (Rf 0.8). Reaction mixture was purified without concentration, utilizing the preparatory Synergi Hydro C18 column and a 4%/min acetonitrile gradient. Lyophilization yielded the desired product (FITC-Tre), 9, as a yellow solid (16 mg, 72%).

### Synthesis of Quantum Dot-Labelled Tre (QD-Tre), Compound 66

### (1-Deoxy-α-D-gluco-hept-2-ulopyranosyl 2-N-isothiocyanate-2-deoxy-α-D-glucopyranoside)

Compound 7 (2.5 mg, 0.007 mmol, 1 eq) was dissolved in 75 mM NaHCO₃ buffer pH 9 (200 μl). To this was added thiophosgene as a solution (20 μl of thiophosgene into 1 mL of chloroform). 100 μl of thiophosgene solution was added to reaction (3 mg, 0.26, 4.2 eq). The resulting biphasic mixture was stirred at room temperature for 3 hours upon which TLC (5 ethanol:3 NH₄OH:1 water) showed complete consumption of starting material (Rf 0.2) and conversion to a single product (Rf 0.65). Excess thiophosgene and chloroform were removed in vacuo and crude product 64 was used without further purification.

An 8 μM solution of CdSe—ZnS (50 μl) core-shell quantum dots (emission λmax 655 nm) in borate buffer at pH 8 (Invitrogen) was buffer exchanged into water by repeated (×5) centrifugal filtration through a 10 kDa cutoff spin filter. The quantum dots were then resuspended in water. 50 μl of this 8 μM quantum dot solution was then added to the solution of 64, and the total volume made up to 1.0 mL with 75 mM NaHCO₃ buffer at pH 9.0 (pH electrode). Reaction mixture was shaken at 4° C. for 14 hours. Excess sugar and salt was removed from the reaction mixture by size exclusion chromatography (PD 10 column, Amersham) with water as the mobile phase. The quantum dot solution was concentrated to 1 mL, using 10 Kda spin filter, and the concentration determined using previously reported procedures²² to be 0.44 μM (8350=3880000 M⁻¹ cm⁻¹). The modification of the quantum dots was confirmed using an agarose gel

The carbohydrate loading on the quantum dots was determined using the phenol sulphuric acid method. An aliquot of the quantum dot solution (50 μl) was treated with concentrated sulphuric acid (75 μl) and aqueous phenol (5% w/w, 10 μl) and heated to 90° C. After 5 minutes the sample was cooled to room temperature and A490 measured, referenced to a solution of carbohydrate modified quantum dots and acid. The concentration of trehalose was determined by comparison to a standardised curve. The carbohydrate content per dot was calculated from the ratio of trehalose concentration to the concentration of ZnS—CdSe quantum dots and was found to be ˜110 sugars/dot.

### Experiment 1

### Activity as Substrate for Ag85 A, B and C Transesterase Reactions

Mono and dihexanoyl esters of Tre were used to probe the rate of transesterification with the labelled and optionally derivatised Tre probe molecules (herein Tre*) in the presence of Ag85 enzymes. The rate was compared to that of the reaction of the mono/dihexanoyl Tre ester with unlabelled and underivatised Tre. Products were analysed by Mass Spectrometry (MS).

### MS Kinetic Parameter Determination:

A Waters QuattroMicro-MS with electrospray ionization operating in negative mode (ESI−) was interfaced with a Waters 1525 g HPLC system and Waters 2777 sampler fitted with a 4-port injector module. MS analysis was under the control of Micromass Masslynx 4.1 software, and data were processed using Masslynx4.1, QuantLynx, Microsoft Excel 2003 SigmaPlot 11.0 and Origin 7.5. The HPLC/auto-sampler control was divided into two stages: a) injection of the internal standard into valve 1 and b) injection of the analyte solution into valve 2. Both valves were switched simultaneously and the analyte and standard mixed in a 186 μL peek loop before elution directly onto the source. Mobile phase was CH₃CN:H₂O (50:50 volume); flow rate: 0.2 mL/min; isocratic method for 3 min; injection volume: 10.0 μL, electrospray negative; ESI− (single ion monitoring) for 3 min; Single ion peaks monitored.

### Typical Procedure for Standard Curve Determination

All reactions were performed in TEA (triethanolamine) buffer (1 mM, pH 7.2) at 37° C. Eight samples were analysed with one containing 20 μM n-acetyl-glucosamine (GlcNAc) in TEA buffer only, and the other seven containing fixed concentrations of trehalose (5, 10, 20, 40, 80, 120 and 160 μM), di-hexanoyl ester (X) (5, 10, 20, 40, 80, 120 and 160 μM), mono-hexanoyl ester (0.5, 1, 2, 4, 8, 12 and 16 μM) and hexanoic acid (0.5, 1, 2, 4, 8, 12 and 16 μM) in 1 mM TEA, pH 7.2. The samples were injected as described above. The total ion count peak area was measured for each compound after normalisation for ionization efficiency using the standard. Plots were constructed using QuantLynx of response against concentration and the slope was used to obtain concentration information during subsequent reactions.

### Typical Procedure for Kinetic Parameter Determination

Eight vials were prepared, one containing 20 μM GlcNAc (1 mM TEA, pH 7.2), the others containing fixed final concentrations of di-hexyl trehalose (500 μM) with varying final concentrations of trehalose (10, 15, 25, 50, 75, 100, 250 uM). Ag85C (204) was added to a vial to a final concentration of 50 nM and the concentrations of substrates and products were monitored. Initial rates were calculated and the data were fit to the relevant rate equation using “Origin 7.5”.

### Typical Procedure for Substrate Screen

Upon initiation of reactions by addition of enzyme (or buffer in the case of control wells), each well in a 96-well plate contained 500 μM di-Hexanoyl ester, 500 μM substrate and 100 nM Ag85 (A, B or C) in 1 mM TEA buffer (pH 7.2). Plates were incubated at 37° C. for timed intervals and the formation of products was determined by MS analysis (continuum scan from 100-900 Da). The ratio of peak intensity of substrate to product was used as a qualitative indication of activity. In all cases control solutions without enzyme were used to evaluate the presence of uncatalysed background reaction. Results are shown in Table 1 below.

### Experiment 2

### Uptake of ¹⁴C-Labelled Tre

¹⁴C-Sugar Uptake into Mtb

H37Rv Mtb cells were grown in 7H9 media and were harvested at an OD600 of 0.5 by centrifugation (1250 g at 4° C. for 10 min), washed once with buffer (Hepes 25 mM and 0.05M Tween at pH 7.2) and then resuspended in the same buffer. Radio-labelled ¹⁴C-trehalose (0.1 μCi/ml, obtained from ARC chemicals), glycerol (1 μCi/ml), and corresponding nonlabelled sugars (50 mM) were mixed and added to the 16 ml cell suspension. Trehalose was used at a tenfold lower concentration of radioactivity (0.1 μCi/ml) relative to glucose and arabinose, (1 μCi/ml) whose uptake was also separately studied. 1 ml aliquots were removed in at 15, 30, 45, 60 and 120 minutes and were filtered through GA-4 Metricel filter membranes (diameter, 2.54 cm; pore size, 0.8 μm; Gelman Instrument Co., Ann Arbor, Mich.) The membranes were washed 3×2 ml with hepes/tween buffer and 1×2 ml LiCl 0.1M. Filter papers were then placed in ultimax gold scintillation fluid. Scintillation counting was conducted for 2 minutes each vial. All experiments were conducted in triplicate and samples without sugar were used as control. Uptake experiment was repeated over a 24 hour time period with ¹⁴C-glucose (0.9 μCi/ml) ¹⁴C-Trehalose (0.6 μCi/ml) in 7H9 media, which contain 10 mM glucose. No unlabeled trehalose was added to the experiment. 1 ml aliquots were removed and counted as before with time points at 40, 90 mins, 4 and 24 hours.

Compounds 1 to 24 are as shown in FIG. 5. Results represent the ratio of the peak height of mono-hexanoyl ester of labelled or derivatised trehalose to the peak height of labelled or derivatised Tre.

TABLE 1
Relative substrate response towards transesterification.

| Compound | Ag85A | Ag85B | Ag85C |
|---|---|---|---|
| 1 | 100 | 100 | 100 |
| 2 | 32 | 13 | 111 |
| 3 | 73 | 1415 | 102 |
| 4 | 12 | 7 | 57 |
| 5 | 19 | 13 | 64 |
| 6 | 7 | 8 | 76 |
| 7 | 47 | 56 | 91 |
| 8 | 25 | 14 | 81 |
| 9 | 229 | 38 | 44 |
| 10 | 50 | 16 | 129 |
| 11 | 1 | 4 | 1 |
| 12 | 148 | 54 | 166 |
| 13 | 66 | 24 | 98 |
| 14 | 7 | 35 | 82 |
| 15 | 6 | 4 | 28 |
| 16 | 37 | 24 | 113 |
| 17 | 18 | 17 | 94 |
| 18 | 37 | 13 | 64 |
| 19 | 102 | 92 | 99 |
| 20 | 149 | 412 | 91 |
| 21 | 229 | 412 | 91 |
| 22 | 115 | 156 | 274 |
| 23 | 18 | 19 | 45 |
| 24 | 6 | 28 | 8 |

¹⁴C Uptake into Infected Macrophages

Murine J774 Macrophage cells grown in HMEM media were split into two bottles and allowed to adhere for two days, upon which time confluence was determined. Cells were at 5×10⁷ density. Media was exchanged for fresh and one bottle of cells was infected with 5×10⁸ H37Rv bacteria. After three hours, cells were washed with HMEM and allowed to incubate at 37° C. overnight. Following 24 hours of incubation, media was exchanged for fresh media and ¹⁴C-Trehalose 10 μCi in 100 μl ethanol was added to both infected and uninfected cultures. Cells were allowed to incubate with trehalose for 24 hours. Upon completion media was removed and pelleted by centrifugation, as it contained floating Mtb. Macrophages were gently washed with HMEM media and lysed with SDS 0.1% in 10 ml of media. Cells were further washed with 2×5 ml PBS buffer 10 give a final concentration of 0.05% SDS and lysate was collected in falcon tubes. Lysate was vortexed for ca 1 min. Once the solution was clear, lysate was centrifuged at 3600 rpm for 20 minutes and supernatant was poured off and collected for scintillation counting. Additionally, floating Mtb, pelleted Mtb, as well as controls, were treated to the same following conditions. Pellet (or pellets made from floating Mtb) was resuspended and transferred to a 1.5 ml screw top ependorf in 1 ml tween and treated with four wash cycles of pelleting and resuspension in 800 μl Tween. Finally, pellet was resuspended in a minimal amount of buffer (200 μl) and added to scintillation fluid.

### ¹⁴C-Tre Lipid Extractions

Lipid extractions from bacteria treated with ¹⁴C-trehalose and were conducted as reported by Slayden et al (2001), Vol. 54, pp. 229. and analyzed by radiographic TLC.

### Experiment 3

### Uptake of FITC-Tre

FITC-Tre Uptake into Mtb

To 2×0.5 ml Mtb in Middlebrook 7H9 media in eppendorf tubes at an OD600 of 0.6 was added FITC-Tre in ethanol, at a final concentration of 400 μM. Probe (FITC-Tre) was also added to 2×0.5 ml heat killed bacteria. Mtb was incubated at 37° C. At 2 hours, one of each live and heat killed samples were pelleted 1 minute at 12000 rpms and resuspended 4 times in order to remove any free probe. Upon washing, bacteria was resuspended in 200 μl media. 2×80 μl of this was added to two wells on a black 96 well plate and fluorescence excitation at 480 nm and emission at 520 nm was read. 24 hour timepoints were treated analogously.

### FITC-Tre Lipid Extractions

These were carried out in the same way as for the ¹⁴C-Tre lipid extractions. FITC-Tre lipid extractions were cospotted with ¹⁴C-Tre extracts in order to compare retention values of FITC and ¹⁴C labeled glycolipids.

FITC-Tre Uptake into Infected Macrophages and Microscopy.

### Antibody Fixing:

J774 Macrophages were grown to confluency on coverslips and then were infected with H37Rv Mtb (2-3 bacteria/macrophage). After 4 hours infection time, macrophages were washed to remove free bacteria and FITC-Tre was added in ethanol to desired concentrations (1 μM, 10 μM, 50 μM, 100 μM). Quantum dots (QDs) were treated in an analogous fashion and were added to a concentration of dots of (7 nm) with roughly 110 sugars/dot (1 μM effective sugar concentration) Uncoated QDs were utilized as a control. RFP expressing BCG was also treated in an identical fashion.

Cells were fixed at different timepoints utilizing the following procedure: Media was removed and cells were washed in PBS Buffer. Cells were then fixed in an aqueous solution of 5% formalin is phosphate buffered saline (PBS) for 15 minutes and then washed again using two 2-minute treatments with PBS (0.5 ml) for 2 min. Cells were permeabalized with 0.1% triton X-100 in PBS for 5 minutes at room temperature. Triton was washed way using 3 separate 2 minute treatments with PBS (0.5 ml). Non-specific protein interactions were blocked with protein blocker (1 ml) with 1 drop goat serum for 1 hour at RT. Primary Mycobacterium tuberculosis antibody (ab905) was obtained from abcam and was labeled utilizing Zenon Rabbit Polyclonal IgG labeling kit from Molecular Probes. Antibody and zenon Fab fragments were prepared according to kit specifications and was diluted to 1 ml in PBS with goat serum. Coverslips were incubated with antibody solution for 30 minutes at 37° C., were then washed with PBS and 0.1% tween 20 (3×5 min) and were fixed in 5% formalin for 15 minutes. Coverslips were drained and mounted in anti-fade media (VECTASHIELD® Mounting Medium).

Cells treated with FITC-Tre and no antibody were exposed to the same mounting procedure, except second fixation step was omitted. For cells labeled with DAPI (4′,6-diamidino-2-phenylindole), a 0.1 mg/ml stock solution of DAPI was made up in PBS buffer and cells were incubated with 1 μg/ml DAPI solution for 5 minutes immediately prior to mounting.

### Microscopy

Images of stained cells were obtained by confocal microscopy (Leica SP2, Leica Microsystems, Exton, Pa.) using a 63× oil immersion objective NA 1.4. Multiple fields were sampled, and representative images were recorded. Fluorescein was excited using a 496 nm resulting in emission at 502 nm-565 nm. RFP Alexa 594-labeled Mtb antibody and Quantum dots were excited using a 556 nm laser, with emission at 594 nm-665 nm. Images were gathered sequentially and stacked when DAPI was used to label cell so as to minimize cross-talk between channels. DAPI was first excited at 405 nm and emission spectrum was recorded (416 nm-482 nm) before the other fluorochromes were excited and emission spectrums recorded. Essential Sequential Z sections of stained cells were also recorded for generation of stacked images through cell. All data were processed with Leica LAS AF Lite and compiled in Adobe Illustrator.

### Schemes, Synthesis and Characterization

### Ketoside Trehalose Synthesis

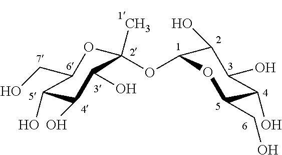

Ketoside trehalose analogues are numbered in the supplementary information in the following manner. This numbering follows the precedent of analog number set by IUPAC nomenclature for ketosides. In the main text, for clarity, compounds are are referred to as methyl-trehalose and are numbered according to the convention for unmodified trehalose.

Compound routes shown in Schemes S2-S23: 25⁶, 27⁷, 28⁸, 29⁹, 30¹⁰, 31^{11,12}, 32¹³, 33^{11,14} 45^{15,16}, 48¹⁶, 51¹⁷, 53¹⁸, 54¹⁸, and 55¹⁹ and were synthesized as has been recorded previously and their characterization matched previously reported spectroscopic data.

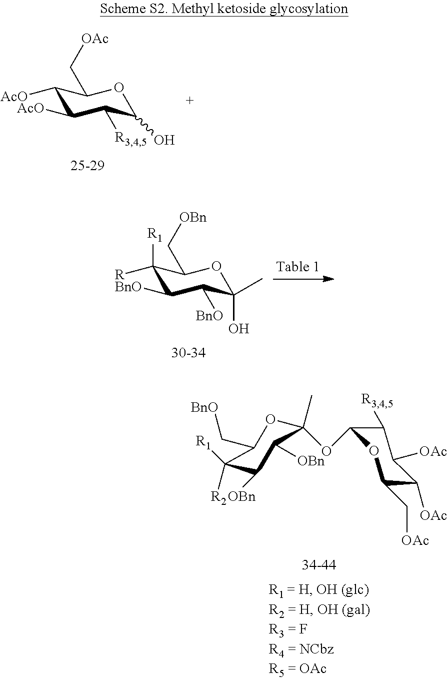

TABLE S1
Glycosylation conditions

| | | | | | | T | | | | |
|---|---|---|---|---|---|---|---|---|---|---|
| Entry | Donor | mmol | Acceptor | mmol | Catalyst | ° C. | Time | | Yield | α,α:α,β |
| 1 | 33 | 0.31 | 29 | 0.58 | TMSOTf | −78 | 30 | min | 30% | 3:7 |
| 2 | 31 | 0.04 | 29 | 0.09 | TMSOTf | −78 | 30 | min | 95% | 1:1 |
| 3 | 30 | 0.24 | 25 | 0.25 | TMSOTf | −40 | 3 | h | 93% | 7:1 |
| 4 | 30 | 0.14 | 26 | 0.22 | TMSOTf | −40 | 2 | h | 82% | 6:1 |
| 5 | 32 | 0.32 | 25 | 0.42 | TMSOTf | −40 | 30 | min | 88% | 5:1 |
| 6 | 32 | 0.14 | 26 | 0.16 | TMSOTf | −40 | 1 | h | 90% | 6:1 |
| 7 | 30 | 0.21 | 28 | 0.18 | TMSOTf | −40 | 3 | h | 52% | 4:1 |
| 8 | 30 | 0.31 | 27 | 0.38 | TMSOTf | −78 | 15 | min | 81% | 6:1 |

Acceptors 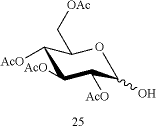 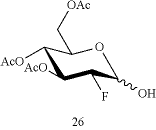 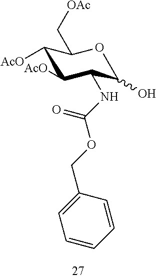 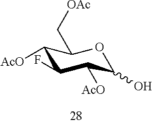 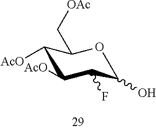
Donors

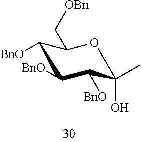

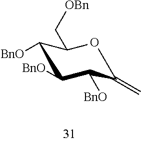

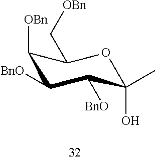

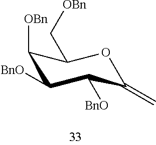

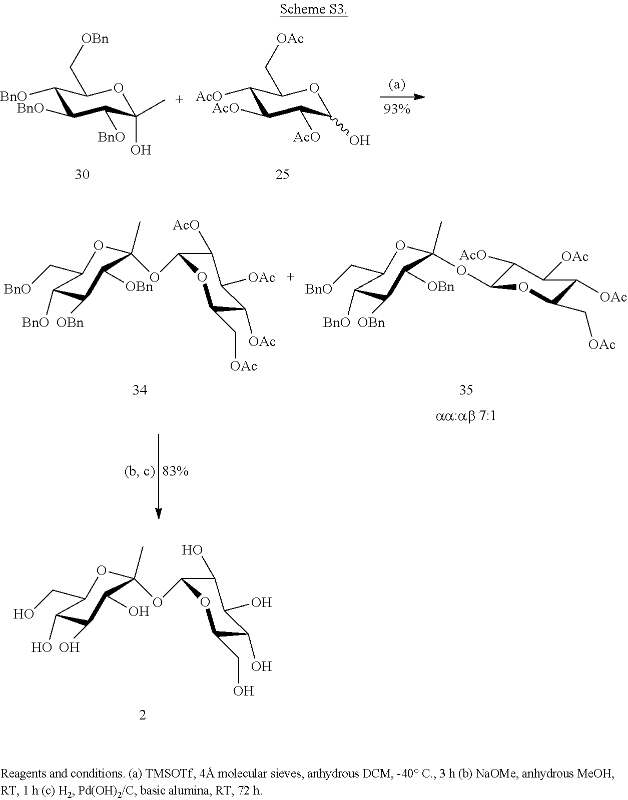

### 2,3,4,6-Tetra-O-benzyl-1-deoxy-α-D-gluco-hept-2-ulopyranosyl-(2→1)-2,3,4,6-tetra-O-acetyl-α-D-glucopyranoside (34)

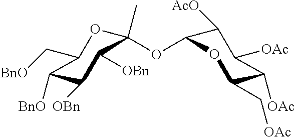

30¹⁰ (134 mg, 0.24 mmol, 1 eq) and 25⁶ (87 mg, 0.25 mmol, 1.05 eq) were dried in vacuo for 1 hour and then dissolved in anhydrous CH₂Cl₂ (7 mL) and added to a dry flask in the presence of molecular sieves (ca. 100 mg). To this was added TMSOTf (10 μl, 0.054 mmol, 0.2 eq) at −40° C. in the under an Ar atmosphere. The resulting mixture was stirred for 3 h. (2.5:1 petrol/ethyl acetate) revealed the production of two new product spots (R_{f} 0.3) and (R_{f} 0.25) and complete consumption of starting material. The reaction was then quenched by the addition 0.01 mL triethylamine, filtered through Celite® and concentrated. The crude product was purified by column chromatography (5:2 petrol/ethyl acetate) to give colorless oil (189.5 mg, 93%) as a mixture of α,α and α,β products (7:1) that were separated during a second round of column chromatography (3:1 petrol/ethyl acetate) to afford pure the desired compound (162 mg, 85.6%) as a clear oil.

[α]_{D}²⁴+80.9 (c=1 in CHCl₃); ¹H NMR (400 MHz, CHLOROFORM-d) δ ppm 1.46 (3H, s, C-1′), 1.96 (3H, s, 1×OCOCH₃), 2.01 (6H, s, 2×OCOCH₃), 2.05 (3H, s, 1×OCOCH₃), 3.32 (1H, d, J_{3′4′}=9.6 Hz, H-3′), 3.56-3.64 (3H, m, H-7_{a′}, H-7_{b}′, H-5′), 3.82 (1H, dd, J_{6a,6b}=12.4 Hz, J_{5,6a}=1.9 Hz, H-6ₐ), 3.88 (1H, ddd, J_{5,6}=10.1 Hz, J_{6,7a}=3.8 Hz, J_{6,7b}=2.5 Hz H-6′), 4.05-4.11 (2H, m, H-4′, H-6_{b}), 4.33 (1 H, ddd, J_{4,5}=10.4 Hz J_{5,6a}=4.3 Hz, J_{5,6b}=2.0 Hz, H-5), 4.48 (1H, d, J=12.3 Hz, 1×OCH₂Ph), 4.56 (1H, d, J=12.4 Hz, 1×OCH₂Ph), 4.56 (1H, d, J=11.0 Hz, 1×OCH₂Ph), 4.62 (1H, d, J=11.3 Hz 1×OCH₂Ph), 4.85 (1H, d, J=11.1 Hz 1×OCH₂Ph), 4.92 (1H, d, J=10.9 Hz, 1×OCH₂Ph), 4.95-5.07 (4H, m, H-4, 2×OCH₂Ph, H-2), 5.37 (1H, d, J_{1,2}=3.6 Hz, H-1), 5.55 (1H, at, J_{2,3}=J_{3,4}=9.7 Hz, H-3), 7.08-7.40 (20H, m, Ar—H); ¹³C NMR (126 MHz, CHLOROFORM-d) δ ppm 20.6, 20.6, 20.7, 20.7, (4×OCOCH₃) 22.6 (C-1′), 61.6 (C-6), 67.1 (C-5), 68.1 (C-7′), 68.6 (C-4), 70.2 (C-2), 70.7 (C-3), 72.0 (C-6′), 73.5 (OCH₂Ph), 74.8 (OCH₂Ph), 75.5 (2×OCH₂Ph), 78.3 (C-5′), 82.6 (C-4′), 84.5 (C-3′), 89.2 (C-1), 101.4 (C-2′), 127.5, 127.6, 127.7, 127.9, 128.3, 128.3, 128.4, 128.8, 130.8 (4×OCH₂Ph), 138.0, 138.2, 138.3, 138.5 (4×1C, 4×OCH₂Ph), 169.6, 169.8, 170.2, 170.5 (4×C═O); IR (thin film): ν=3063.5, 3030.6, 2925.4, 1753.9 (C═O), 1496.9, 1454.2, 1367.4, 1222.3, 1133.1, 1090.4, 1038.06, 956.5, 916.6, 873.0, 737.4 cm⁻¹; MS m/z (ESI⁺) 902.4 (M+NH₄⁺); HRMS (ESI⁺) calcd. for C₄₇H₅₃O₁₃ (M+Na⁺): 907.3517. Found: 907.3512.

### 2,3,4,6-Tetra-O-benzyl-1-deoxy-α-D-gluco-hept-2-ulopyranosyl-(2->1)-2,3,4,6-tetra-O-acetyl-β-D-glucopyranoside (35)

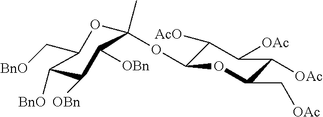

The titled compound was purified as the lower spot by TLC (2:1 petrol/ethyl acetate) (R_{f} 0.25) from the reaction between 30 (134.4 mg, 0.242 mmol, 1 eq) and 25⁶ (87.2 mg, 0.25 mmol, 1.05 eq) to produce 35 as a clear oil. (27 mg, 14.3%)

[α]_{D}²⁴+17.7 (c=1 in CHCl₃); ¹H NMR (500 MHz, CHLOROFORM-d) δ ppm 1.43 (3H, s, C-1′), 1.96 (1H, s, 1×OCOCH₃), 2.00 (5H, s, 5×OCOCH₃), 2.03 (5 H, m, 5×OCOCH₃), 2.04 (1H, s, 1×OCOCH₃), 3.34 (1H, d, J_{3′,4′}=9.6 Hz, H-3′), 3.61 (1H, ddd, J_{4,5}=12.4 Hz, J_{5,6a}=7.3 Hz, J_{5,6b}=2.5 Hz, H-5), 3.59-3.62 (1H, m, H-7_{b}′), 3.69-3.73 (2H, m, H-5′ H-7ₐ′), 4.03-4.07 (2H, m, H-4′, H-6ₐ), 4.12 (1H, dd, J_{6a,6b}=12.4 Hz, J_{5,6b}=4.8 Hz, H-6_{b}), 4.18 (1H, ddd, J_{5′,6′}=6.6 Hz, J_{6′,7a′}=3.0 Hz, J_{6′,7b′}=2.0 Hz, H-6′), 4.48 (1H, d, J=11.9 Hz, 1×OCH₂Ph), 4.53 (1H, d, J=10.9 Hz, 1×OCH₂Ph), 4.59 (1H, dd, J=11.4 Hz, 1×OCH₂Ph), 4.61 (1H, dd, J=12.3 Hz, 1×OCH₂Ph), 4.84 (1H, d, J=10.86 Hz, 1×OCH₂Ph), 4.89 (1H, d, J_{1,2}=8.2 Hz, H-1), 4.89 (2H, d, J=10.4 Hz, 2×OCH₂Ph), 4.94 (1H, d, J=11.1 Hz, 1×OCH₂Ph), 5.00-5.06 (2H, m, H-4, H-2), 5.19 (1H, at, J_{2,3}=J_{3,4}=9.5 Hz, H-3), 7.16-7.18 (2H, m, Ar—H), 7.27-7.35 (18H, m, Ar—H); ¹³C NMR (500 MHz, CHLOROFORM-d) δ ppm 20.3, 20.6, 20.6, 20.7 (4×OCOCH₃), 22.1 (C-1′), 62.0 (C-6), 68.4 (C-4), 68.5 (C-7′), 70.9 (C-2), 72.0 (C-6′ or C-5), 72.4 (C-6′ or C-5), 72.9 (C-3), 73.4 (OCH₂Ph), 74.9 (OCH₂Ph), 75.5 (OCH₂Ph), 75.5 (OCH₂Ph), 78.2 (C-5′), 82.5 (C-4′), 84.5 (C-3′), 94.6 (C-1), 102.4 (C-2′), 127.5, 127.5, 127.6, 127.6, 127.8, 127.8, 128.3, 128.3, 128.4 (4×OCH₂Ph), 138.1, 138.2, 138.4, 138.6 (4×1C, 4×OCH₂Ph), 169.0, 169.4, 170.3, 170.6 (4×C═O); IR (thin film): ν=3029, 2931, 1755 (C═O), 1496, 1454, 1366, 1230, 1211, 1038, 908, 737 cm⁻¹; MS m/z (ESI⁺) 902.4 (M+NH₄⁺); HRMS (ESI⁺) calcd. for C₄₇H₅₃O₁₃ (M+Na⁺): 907.3517. Found: 907.3512

### 1-Deoxy-α-D-gluco-hept-2-ulopyranosyl-(2→1)-α-D-glucopyranoside (2)²⁰

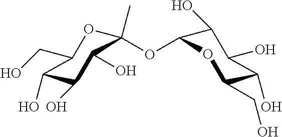

34 (120 mg, 0.13 mmol) was dissolved in 15 mL methanol with NaOMe (30 mg, 0.55 mmol, 4 eq) and stirred at room temperature for 1 hour until complete disappearance of starting material (R_{f} 0.55) and appearance of a new spot (R_{f} 0.05) was observed by TLC (1:1 petrol/ethyl acetate) and deacetylated sugar was detected by ESI⁺ m/z (M+Na⁺): 741.3. Reaction was neutralized with DOWEX 50WX8 (H⁺ form) ion exchange resin and concentrated in vacuo. The clear oil was redissolved in ethanol (15 mL) and to this was added basic alumina (70.1 mg) and 20% Pd(OH)₂/C (80 mg) and palladium was activated by repeat purge flush cycles with hydrogen. Reaction was stirred under hydrogen atmosphere (balloon) at room temperature. After 72 h reaction was filtered through filter paper and filtrate was evaporated and purified by column chromatograph (9:5 ethyl acetate/methanol) to give the desired, fully deprotected sugar, as a clear oil (40 mg, 83%).

[α]_{D}²⁵+121.2 (0.32 in MeOH); [Lit. [α]_{D}²⁵+140.0 (c=0.83 in MeOH)]²⁰; ¹H NMR (400 MHz, DEUTERIUM OXIDE) δ ppm 1.47 (3H, s, C-1′), 3.24 (1H, d, J_{3,4} 9.9 Hz, H-3′), 3.35 (2H, at, J_{4,5}=J_{5,6}=9.6 Hz, H-5′, H-4), 3.53 (1H, dd, J_{2,3}=10.0 Hz, J_{1,2}=3.7 Hz, H-2), 3.59 (1H, dd, J_{6a,6b}=12.3 Hz, J_{5,6a}=5.5 Hz), 3.61 (1H, dd, J_{7a,7b}=11.8 Hz, J_{6,7a}=5.1 Hz, H-7a), 3.67 (1H, at, J_{3,4}=J_{4,5}=10.8 Hz, H-4), 3.73-3.83 (5H, m, H-6b, H-5, H-3, H-4′ H-7b′), 3.99 (1H, ddd, J_{5,6}=10.1 Hz, J_{6,7a}=5.3 Hz, J_{6,7b}=2.3 Hz, H-6′), 5.22 (1H, d, J_{1,2}=3.8 Hz, H-1); ¹³C NMR (126 MHz, DEUTERIUM OXIDE) δ ppm 22.5 (C-1′), 60.5 (C-6), 60.5 (C-7), 69.8 (C-4), 69.9 (C-5′), 71.4 (C-5), 71.8 (C-2), 72.3 (C-6′), 72.5 (C-3), 72.8 (1C-4′), 76.4 (C-3′), 91.5 (C-1), 101.0 (C-2′); MS m/z (ESI⁺) 379.2 (M+Na⁺); Spectroscopic data matches previously reported data²⁰

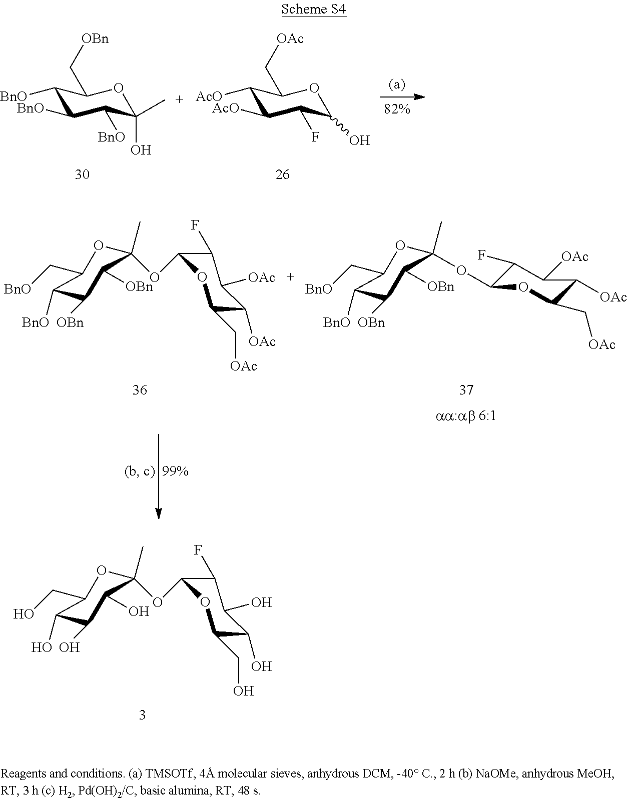

### 2,3,4,6-Tetra-O-benzyl-1-deoxy-α-D-gluco-hept-2-ulopyranosyl-(2→1)-3,4,6-tri-O-acetyl-2-deoxy-2-fluoro-α-D-glucopyranoside (36)

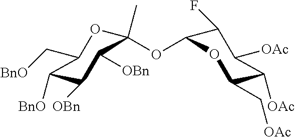

30 (81 mg, 0.15 mmol, 1 eq) and 26 (69 mg, 0.22 mmol, 1.5 eq) were dried in vacuo for 1 hour and then dissolved in anhydrous CH₂Cl₂ (7 mL) and added to a dry flask in the presence of molecular sieves (ca. 100 mg). To this was added TMSOTf (10 μl, 0.054 mmol, 0.3 eq) at −40° C. under an Ar atmosphere. The resulting mixture was stirred for 2 h upon which time TLC (2.5:1 petrol/ethyl acetate) revealed the production of two very close new product spots (R_{f} 0.3) and (R_{f} 0.27) and complete consumption of starting material. The reaction was then quenched by the addition 0.01 mL triethylamine, filtered through Celite® and concentrated to produce. The crude product was purified by column chromatography (2:1 etrol/ethyl acetate) to give the desired compound as a colorless oil (104 mg, 82%) and a 6:1 mixture of α,α (89.1 mg) to α,β. None of the α,β product was obtained to purity.

[α]_{D}²⁴+65.5 (c=1.0 in CHCl₃); ¹H NMR (500 MHz, CHLOROFORM-d) δ ppm 1.48 (3H, s, C-1′), 2.01 (3H, s, 1×OCOCH₃), 2.06 (3H, s, 1×OCOCH₃), 2.08 (3H, s, 1×OCOCH₃), 3.35 (1H, d, J_{3′,4′}=9.4 Hz, H-3′), 3.62 (1H, m, J_{7a,7b}=11.0 Hz, J_{6,7a}=1.6 Hz, H-7b), 3.65 (1H, at, J_{5,6}=J_{4,5}=9.8 Hz, H-5′), 3.71 (1H, d, J_{7a,7b}=11.3 Hz, J_{6,7a}=4.1 Hz, H-7a), 3.83 (1H, dd, J_{6b,6a}=12.5 Hz, J_{6b,5}=2.0 Hz, H-6b), 3.97 (1H, ddd, J_{5′,6′}=9.9 Hz, J_{6′,7a′}=2.0 Hz, J_{6′,7b′}=1.7 Hz, H-6′), 4.06 (1H, at, J_{3,4}=J_{4,5}=9.4 Hz, H-4′), 4.08 (1H, dd, J_{6a,6b}=12.3 Hz, J_{6a,6b}=4.5 Hz, H-6a), 4.33 (1H, ddd, 4,5=10.4 Hz, J_{5,6a}=4.7 Hz, J_{5,6b}=2.2 Hz, H-5), 4.49 (1H, d, J=12.3 Hz, 1×OCH₂Ph),), 4.54 (6H, ddd, J_{2,F}=48.0 Hz, J_{2,3}=9.8 Hz, J_{1,2}=3.8 Hz, H-2), 4.57 (1H, d, J=11.1 Hz, 1×OCH₂Ph), 4.60 (1H, d, J=12.3 Hz, 1×OCH₂Ph), 4.62 (1H, d, J=10.4 Hz, 1×OCH₂Ph), 4.83 (1H, d, J=10.9 Hz, 1×OCH₂Ph), 4.93 (1H, d, J=11.4 Hz, 1×OCH₂Ph), 4.96 (1H, d, J=12.3 Hz, 1×OCH₂Ph), 4.99 (1H, d, J=11.4 Hz, 1×OCH₂Ph) 4.99 (1H, dt, J_{4,5}=J_{3,4}=9.6 Hz, J_{4,F}=3.0 Hz), 5.46 (1H, d, J_{1,2}=3.9 Hz, H-1), 5.61 (1H, dt, J_{3,F}=12.2 Hz, J_{3,4}=9.4 Hz, J_{2,3}=9.4 Hz H-3), 7.16-7.37 (20H, m, Ar—H); ¹³C NMR (126 MHz, CHLOROFORM-d) δ ppm 20.6, 20.6, 20.8 (3×OCOCH₃), 22.9 (C-1′), 61.6 (C-6), 67.0 (C-5), 68.2 (C-4), 68.5 (C-7′), 71.3 (1C, d, J_{C-3,F}=26.5 Hz, C-3) 71.3 (C-6′), 73.1 (OCH₂Ph), 74.6 (OCH₂Ph), 75.5 (OCH₂Ph), 75.5 (OCH₂Ph), 78.2 (C-5′), 82.6 (C-4′), 84.5 (C-3′), 87.2 (1C, d, J_{C-2,F}=195.5 Hz, C-2), 88.8 (1C, d, J_{C-1,F}=21 Hz, C-1), 101.5 (C-2′), 127.5, 127.5, 127.6, 127.7, 127.8, 127.9, 127.9, 128.2, 128.3, 128.4 (4×OCH₂Ph), 138.2, 138.4, 138.6 (4×1C, 4×OCH₂Ph), 169.7, 170.1, 170.5 (3×C═O); ¹⁹F NMR (1H) (377 MHz, CHLOROFORM-d) δ ppm −197.5; ν=3064, 3030, 2940, 1754 (C═O), 1605, 1540, 1497, 1454, 1366, 1223, 1132, 1089, 1064, 974, 949, 912, 872, 751, 737 cm⁻¹; MS m/z (ESI⁺) 844.35 (M+NH₄⁺); HRMS (ESI⁺) calcd. for C₄₇H₅₃FO₁₃ (M+Na⁺) 867.3365. Found: 867.3362.

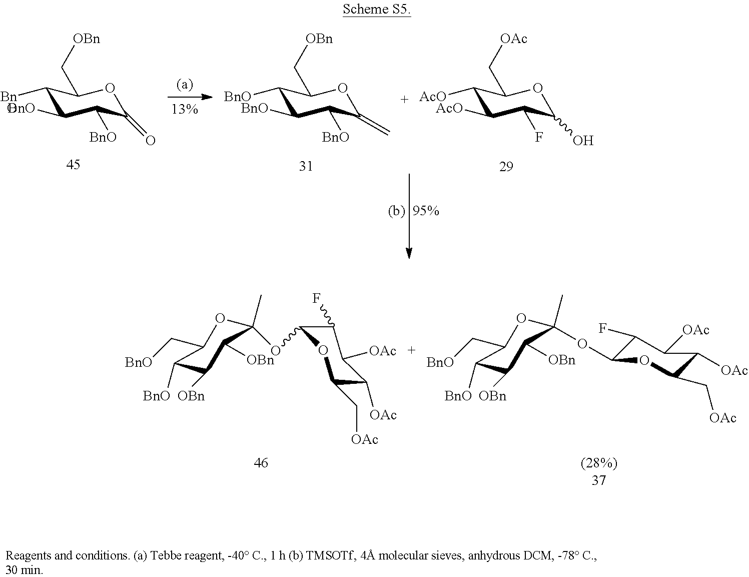

### 2,3,4,6-Tetra-O-benzyl-1-deoxy-α-D-gluco-hept-2-ulopyranosyl-(2→1)-3,4,6-tri-O-acetyl-2-deoxy-2-fluoro-β-D-glucopyranoside (37)

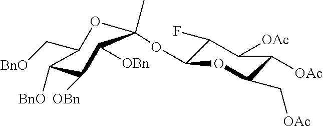

Following the procedure in Li, 2001¹³ a solution of 31 (24 mg, 0.045 mmol, 1 eq) and 29 (29 mg, 0.094 mmol, 2 eq) and molecular sieves 4A (MS 4A) (100 mg) in dry CH₂Cl₂ (5 mL) was stirred under nitrogen atmosphere at room temperature for 30 min. The solution was cooled to −78° C. and TMSOTf (5 μL, 0.027 mmol, 0.5 eq) was added. The reaction mixture was stirred at −78° C. for 30 minutes. The reaction was monitored by TLC (2.5:1 petrol/ethyl acetate) and upon completion one broad product spot was visible (R_{f} 0.3) with disappearance of starting glycal 31 (R_{f} 0.8) and reaction was quenched with triethylamine (20 μl) and passed through Celite®. After the removal of the solvent under reduced pressure, the residue was purified by silica gel column chromatography (2.5:1 petrol/ethyl acetate) as the eluent to afford the products (35 mg, 95%) 1:1 mixture of the α,α and α,β and gluco and manno sugars. From these only the titled compound could be isolated to purity as a clear oil (10 mg, 28%).

[α]_{D}²⁴+39.7 (c=0.37 in CHCl₃); ¹H NMR (500 MHz, CHLOROFORM-d) δ ppm 1.42 (3H, C-1′), 2.02 (3H, s, 1×OCOCH₃), 2.05 (3H, s, 1×OCOCH₃), 2.09 (3 H, 1×OCOCH₃), 3.40 (1H, d, J_{3′,4′}=9.8 Hz, H-3′), 3.59 (1H, ddd, J_{4,5}=10.0 Hz, J_{5,6a}=5.1 Hz, J_{5,6b}=2.5 Hz, H-5), 3.63-3.70 (3H, m, H-7a′, H-7b′, H-5′), 4.99-4.11 (2H, m, H-6b, H-4′), 4.09 (1H, dd, J_{6a,6b}=11.8 Hz, J_{6a,6b}=5.5 Hz, H-6a), 4.21 (1H, ddd, J_{5′,6′}=10.1 Hz, J_{6′,7a′}=3.9 Hz, J_{6′,7b′}=1.42 Hz, H-6′), 4.37 (1H, ddd, J_{F,2}=50.0 Hz, J_{2,3}=9.1 Hz, J_{1,2}=8.2 Hz, H-2) 4.50 (1H, d, J=12.0 Hz, 1×OCH₂Ph), 4.54 (1H, d, J=10.7 Hz, 1×OCH₂Ph), 4.59 (1H, d, J=12.0 Hz, 1×OCH₂Ph), 4.71 (1H, d, J=11.7 Hz, 1×OCH₂Ph), 4.85 (1H, d, J=11.0 Hz, 1×OCH₂Ph), 4.87 (1 H, d, J=11.0 Hz 1×OCH₂Ph), 4.91 (1H, d, J=13.3 Hz, 1×OCH₂Ph) 4.94-4.98 (2 H, m, H-4, 1×OCH₂Ph), 5.01 (1H, dd, J_{1,2}=7.7 Hz J_{1,F}=2.4 Hz, H-1), 5.30 (1H, dt, J_{3,F}=14.2 Hz, J_{2,3} 9.2 Hz, J_{3,4} 9.2 Hz, H-3), 7.16-7.34 (20H, m, Ar—H); ¹³C NMR (126 MHz, CHLOROFORM-d) δ ppm 22.3, 22.6, 23.1 (3×OCOCH₃), 28.8 (CH₃), 61.9 (C-6), 68.2 (1C, d, J_{C-4,F}=5.0 Hz, C-4), 68.6 (C-7′), 72.1 (C-5), 72.6 (C-6′), 73.0 (1C, d, J_{C-3,F}=18.9 Hz, C-3), 73.1 (OCH₂Ph), 73.5 (OCH₂Ph), 75.0 (OCH₂Ph), 75.5 (OCH₂Ph), 78.2 (C-5′), 82.7 (C-4′), 84.2 (C-3′), 88.9 (1C, d, J_{C-2,F}=189 Hz, C-2), 94.0 (1C, d, J_{C-1F}=23.9 Hz, C-1), 102.6 (C-2′), 127.5, 127.69, 127.7, 127.7, 127.8, 127.8, 128.0, 128.3, (4×OCH₂Ph), 138.1, 138.6 (4×1C. 4×OCH₂Ph), 169.4, 170.13, 170.5 (3×C═O); ¹⁹F NMR (377 MHz, CHLOROFORM-d) δ ppm −198.52; IR (thin film): ν=2921, 1754 (C═O), 1586, 1549, 1513, 1495, 1453, 1366, 1242, 1196, 1100, 1055, 1029, 735, 698 cm⁻¹; MS m/z (ESI⁺) 844.35 (NH₄⁺); HRMS (ESI⁺) calcd. for C₄₇H₅₃FO₁₃ (M+Na⁺) 867.3365. Found: 867.3362.

### 1-Deoxy-α-D-gluco-hept-2-ulopyranosyl-(2→1)-2-deoxy-2-fluoro-α-D-glucopyranoside (3)

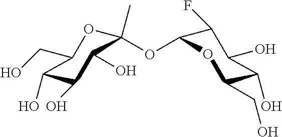

36 (66.6 mg, 0.078 mmol, 1 eq) was dissolved in 15 mL methanol with NaOMe (20 mg, 0.37 mmol, 4.8 eq) and stirred at room temperature for 3 hours until complete disappearance of starting material (R_{f} 0.55) and appearance of a new spot (R_{f} 0.05) was observed by TLC (1:1 petrol/ethyl acetate) and deacetylated sugar was detected by ESI⁺ m/z (M+Na⁺): 741.3. Reaction was neutralized with DOWEX 50WX8 (H⁺ form) ion exchange resin and concentrated in vacuo. The clear oil was re-dissolved in methanol (10 mL) and to this was added basic alumina (5.7 mg) and 20% Pd(OH)₂/C (112 mg) and reaction was stirred under hydrogen atmosphere (balloon) at room temperature. After 48 h. reaction was filtered through filter paper and filtrate was evaporated and purified by column chromatograph (7:3 ethyl acetate/methanol) and Isolute SPE C18 cartridge to give the desired, fully deprotected sugar, as a clear oil (27 mg, 98%).

[α]_{D}²⁵+95.5 (c=0.42 in MeOH); ¹H NMR (500 MHz, DEUTERIUM OXIDE) δ ppm 1.45 (3H, s, C-1′), 3.24 (1H, d, J_{3,4}=9.8 Hz, H-3′), 3.33 (1H, at, J_{4,5}=J_{5,6}=10.1 Hz, H-5′), 3.40 (1H, at, J_{3,4}=J_{4,5}=9.6 Hz, H-4), 3.63 (1H, dd, J_{6a,6b}=11.8 Hz, J_{5,6a}=5.8 Hz, H-6a), 3.66 (1H, dd, J_{7a,7b}=11.9 Hz, J_{6,7a}=5.7 Hz, H-7a′), 3.67 (1H, t, J=9.5 Hz, H-4′), 3.72-3.79 (3H, m, H-6b, H-7b′, H-6′), 3.80 (1H, ddd, J_{4,5}=10.1 Hz, J_{5,6a}=5.0 Hz, J_{5,66}=2.4 Hz, H-5), 4.02 (1H, dt, J_{3,F}=12.9, J_{3,4}=9.4 Hz, J_{2,3}=9.4 Hz, H-3), 4.36 (1H, ddd, J_{2,F}=49.5 Hz, J_{2,3}=9.8 Hz, J_{1,2}=3.8 Hz, H-2), 5.45 (1H, d, J_{1,2}=4.1 Hz, H-1); ¹³C NMR (126 MHz, DEUTERIUM OXIDE) δ ppm 22.27 (C-1′), 60.31 (C-6), 60.59 (C-7), 69.2 (1C, d, J_{C-4,F}=7.5 Hz, C-4), 69.8 (C-5′), 71.2 (1C, d, J_{C-3,F}=17.6 Hz, C-3), 71.9 (C-5), 72.5 (C-6′), 72.9 (C-4′), 76.1 (C-3′), 88.8 (C-1), 89.7 (1C, d, J_{C-2-F}=189 Hz, C-2), 101.1 (C-2′); ¹⁹F NMR (377 MHz, DEUTERIUM OXIDE) δ ppm −197.2 (dt, J_{2,F}=49.3 Hz, J_{1,F}=J_{3,F}=12.6 Hz); MS m/z (ESI⁺) 381.2 (M+Na⁺); HRMS (ESI) calcd. for C₁₃H₂₃FO₁₀ (M+Na⁺): 381.1173. Found: 381.1167.

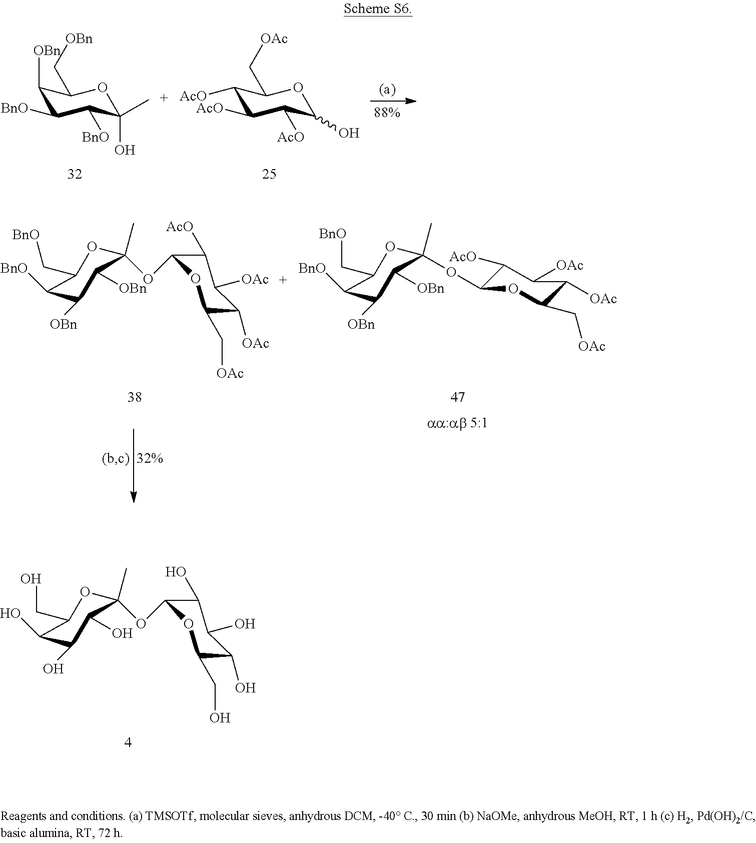

### 2,3,4,6-Tetra-O-benzyl-1-deoxy-α-D-galacto-hept-2-ulopyranosyl-(2→1)-2,3,4,6-tetra-O-acetyl-α-D-glucopyranoside (38)

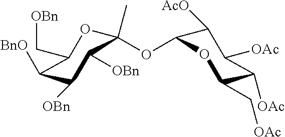

32 (176.1 mg, 0.317 mmol, 1 eq) and 25 (146.2 mg, 0.419 mmol, 1.3 eq) were dried in vacuo for 1 hour and then dissolved in anhydrous CH₂Cl₂ (8 mL) and added to a dry flask in the presence of molecular sieves (ca. 100 mg). To this was added TMSOTf (10 μl, 0.54 mmol, 0.17 eq) at −40° C. in the under an Ar atmosphere. The resulting mixture was stirred for 30 min until TLC (2.5:1 petrol/ethyl acetate) revealed the production of two new product spots (R_{f} 0.3) and (R_{f} 0.27) and complete consumption of starting material. The reaction was then quenched by the addition 0.1 mL triethylamine, filtered through Celite® and concentrated to produce. The crude product was purified by column chromatography (5:2 petrol/ethyl acetate) to give as a colorless oil (246 mg, 88%) as a mixture of α,α and α,β products (5:1) that were separated during further chromatography (3:1 petrol/ethyl acetate) to yield the desired compound as exclusively α,α (180.7 mg, 65%) [α]_{D}²⁴+72.0 (c=1.0 in CHCl₃); ¹H NMR (500 MHz, CHLOROFORM-d) δ ppm 1.46 (3H, s, C-1′), 1.94 (3H, s, 1×OCOCH₃) 2.01 (6H, s, 2×OCOCH₃), 2.04 (3H, s, 1×OCOCH₃), 3.40 (1H, dd, J_{7a,7b} 8.9 Hz, J_{6,7a} 5.3 Hz, H-7b′), 3.56 (1H, dd, J_{7a,7b}=8.4, J_{6,7a}=8.4 Hz, H-7a′), 3.75 (1H, dd, J_{6a,6b}=12.5 Hz, J_{5,6b}=2.2 Hz, H-6b), 3.83 (1H, d, J_{3,4} 9.9 Hz, H-3′), 4.01 (1H, Br s, H-5′), 4.02 (1H, dd, J_{6a,6b}=12.2 Hz, J_{5,6a}=4.2 Hz, H-6a), 4.07 (1 H, dd, J_{4,5}=9.9, J_{5,6}=2.6 Hz, H-5′), 4.10 (1H, ddd, J=7.2, 4.3, 1.2 Hz, H-6′) 4.34 (1H, d, J=11.8 Hz, 1×OCH₂Ph), 4.38 (3H, ddd, J_{4,5}=10.4 Hz, J_{5,6a}=4.3 Hz, J_{5,6b}=2.2 Hz, H-5), 4.40 (1H, d, J=11.6 Hz, 1×OCH₂Ph), 4.57 (1H, d, J=11.6 Hz, 1×OCH₂Ph), 4.61 (1H, d, J=11.1 Hz, 1×OCH₂Ph), 4.82 (1H, Br. s, 1×OCH₂Ph), 4.95 (1H, d, J=11.4 Hz, 1×OCH₂Ph), 4.99 (1H, dd, J_{2,3}=10.2 Hz, J_{1,2}=3.6 Hz, H-2), 5.02 (1H, at, J₁₂=J_{2,3}=10.1, H-2), 5.04 (2H, d, J=10.41 Hz, 1×OCH₂Ph), 5.06 (1H, d, J=10.2 Hz, 1×OCH₂Ph), 5.33 (1H, d, J_{1,2}=3.6 Hz, H-1), 5.53 (1H, at, J_{2,3}=J_{3,4}=9.8 Hz, H-3), 6.85-7.64 (20H, m, Ar—H); ¹³C NMR (126 MHz, CHLOROFORM-d) δ ppm 20.3, 20.6, 20.6, 20.7 (4×OCOCH₃), 22.4 (C-1′), 61.6 (C-6), 67.0 (C-5), 68.4 (C-4), 68.4 (C-7) 70.5 (C-2), 70.5 (2C, s, C-4 and C-6′), 72.6 (OCH₂Ph), 73.5 (OCH₂Ph), 74.2 (C-5′), 74.5 (OCH₂Ph), 75.6 (OCH₂Ph), 80.1 (C-4′), 80.86 (C-3′), 89.3 (C-1), 102.3 (C-2′), 127.5, 127.5, 127.6, 127.7, 127.8, 127.9, 128.1, 128.2, 128.2, 128.3, 128.4 (4×OCH₂Ph), 137.6, 138.4, 138.6, 138.7 (4×1C, 4×OCH₂Ph), 169.6, 169.9, 170.2, 170.6 (4×C═O) IR (thin film): ν=3030.3, 2932.1, 2867.8, 1752.36 (C═O), 1497.0, 1454.4, 1368.0, 1221.2, 1139.1, 1100.0, 1038., 963.61, 922.4, 877.3, 845.4, 772.3, 753.5 cm⁻¹; MS m/z (ESI⁺) 902.4 (M+NH₄); HRMS (ESI⁺) calcd. for C₄₇H₅₃O₁₃Na (M+Na⁺): 907.3517. Found: 907.3511

### 1-Deoxy-α-D-galacto-hept-2-ulopyranosyl-(2→1)-α-D-glucopyranoside (4) ²⁰

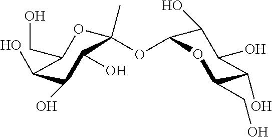

38 (163.2 mg, 0.184 mmol, 1 eq) was dissolved in 15 mL methanol with NaOMe (46.2 mg, 0.855 mmol) and stirred at room temperature for 1 hour until complete disappearance of starting material (R_{f} 0.55) and appearance of a new spot (R_{f} 0.05) was observed by TLC (1:1 petrol/ethyl acetate) and deacetylated sugar was detected by ESI⁺ m/z (M+Na): 739.2. Reaction was neutralized with DOWEX 50WX8 (H⁺ form) ion exchange resin and concentrated in vacuo. The resulting clear oil was redissolved in ethanol (15 mL) and solution was degassed with nitrogen. To this was added basic alumina (50 mg) and 20% Pd(OH)₂/C (70.6 mg) and reaction was stirred under hydrogen atmosphere (balloon) at room temperature. After 72 h. reaction was filtered through filter paper and filtrate was evaporated and purified by column chromatography (9:5 ethyl acetate/methanol) to give the desired, fully deprotected sugar, as a clear oil. (23 mg, 32%).

[α]_{D}²⁵+156.5 (0.46 in MeOH); [Lit. [α]_{D}²²+164.9 (c=0.64 in MeOH)]²⁰; ¹H NMR (500 MHz, CHLOROFORM-d) δ ppm 1.51 (3H, s, C-1′), 3.38 (1H, at, J_{3,4}=J_{4,5}=9.6 Hz, H-4), 3.55 (1H, d, J_{3,4}=10.1 Hz, H-3′), 3.56 (1H, dd, J_{2,3}=10.1 Hz, J_{1,2}=3.8 Hz, H-2), 3.66 (1H, dd, J_{7a,7b}=12.0 Hz, J_{6,7b}=5.4 Hz, H-7b′), 3.69 (1H, dd, J_{7a,7b}=12.0 Hz, J_{6,7b}=7.7 Hz, H-70, 3.71 (1H, dd, J_{6a,6b}=12.6 Hz, J_{5,6a}=5.4 Hz, H-6a), 3.77-3.85 (3H, m, H-3, H-5, H-6b), 3.94 (1H, dd, J_{4,5}=3.5 Hz, J_{5,6}=1.1 Hz, H-5′), 3.95 (1H, dd, J_{3,4}=10.7 Hz, J_{4,5}=3.5 Hz, H-4′), 4.22 (1H, ddd, J_{6,7a}=7.1 Hz, J_{6,7b}=5.0 Hz, J_{5,6}=1.4 Hz, H-6′), 5.26 (1H, d, J_{1,2}=3.8 Hz, H-1); ¹³C NMR (126 MHz, CHLOROFORM-d) δ ppm 25.2 (C-1′), 63.0 (C-6), 63.6 (C-7′), 71.8 (C-5′), 72.0 (C-4′), 72.4 (C-4), 73.8 (C-6′), 73.9 (C-2), 74.2 (C-5), 75.0 (C-3), 75.9 (C-3′), 94.0 (C-1), 103.7 (C-2′); MS m/z (ESI⁺) 379.2 (M+Na⁺). Spectroscopic data matches with that previously reported ²⁰

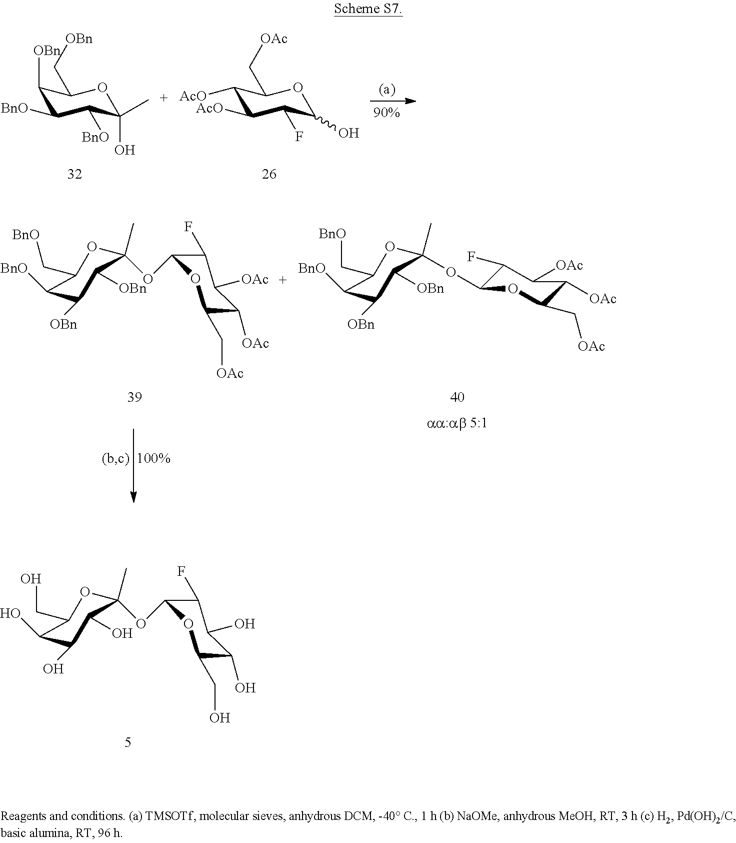

### 2,3,4,6-Tetra-O-benzyl-1-deoxy-α-D-galacto-hept-2-ulopyranosyl-(2→1)-3,4,6-tri-O-acetyl-2-deoxy-2-fluoro-α-D-glucopyranoside (39)

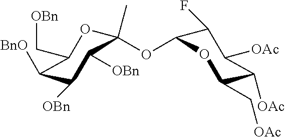

32 (88.7 mg, 0.16 mmol, 1.15 eq) and 26 (44.1 mg, 0.14 mmol, 1 eq) were dried in vacuo for 1 hour then dissolved in anhydrous CH₂Cl₂ (7 mL) and added to a dry flask in the presence of molecular sieves (ca. 100 mg). To this was added TMSOTf (5 μl, 0.027 mmol, 0.18 eq) at −40° C. under an Ar atmosphere. The resulting mixture was stirred for 1 hour upon which time TLC (2.5:1 petrol/ethyl acetate) revealed the production of two new product spots (R_{f} 0.3) and (R_{f} 0.27) and complete consumption of starting material. The reaction was then quenched by the addition 0.01 mL triethylamine, filtered through Celite® and concentrated. The crude product was purified by column chromatography (2:1 petrol/ethyl acetate) to give a 6:1 mixture of the α,α and α,β products (106 mg, 90%). Further purification in (3:1 petrol/ethyl acetate) yielded the desired compound (70.7 mg, 60%) as a colorless oil. α,β product was not obtained to purity through this method

[α]_{D}²⁴+46.6 (c=0.78, CHCl₃); ¹H NMR (500 MHz, CHLOROFORM-d) δ ppm 1.47 (3H, s, C-1′), 2.01 (3H, s, 3×OCOCH₃), 2.06 (3H, s, 3×OCOCH₃), 2.09 (3H, s, 3×OCOCH₃), 3.52 (2H, ad, J=6.6 Hz, H-7a′, H-7b′), 3.83 (2H, dd, J_{6a,6b} 12.5 Hz, J_{5,6b} 2.4 Hz, H-6b), 3.84 (1H, d, J_{3,4}=9.8 Hz, H-3′), 3.98 (1H, dd, J_{4,5}=2.7 Hz, J_{5,6}=1.4 Hz, H-5′), 4.05 (1H, dd, J_{3,4}=9.9 Hz, J_{4,5}=2.7 Hz, H-4′), 4.08-4.15 (2H, m, H-6′, H-6a), 4.33 (1H, ddd, J_{4,5}=10.3 Hz, J_{5,6a}=4.8 Hz, J_{5,6b}=2.2 Hz, H-5), 4.42 (1H, d, J=12.0 Hz, 1×OCH₂Ph), 4.47 (1H, d, J=11.9 Hz, 1×OCH₂Ph), 4.53 (1H, ddd, J_{2,F}=50.8 Hz J_{2,3}=9.6 Hz, J_{1,2}=3.9 Hz, H-2), 4.58 (1H, d, J=11.3 Hz, 1×OCH₂Ph), 4.63 (1H, d, J=11.3 Hz, 1×OCH₂Ph), 4.76 (1H, d, J=11.9 Hz, 1×OCH₂Ph), 4.79 (1H, d, J=12.0 Hz, 1×OCH₂Ph), 4.93 (1H, d, J=11.3 Hz, 1×OCH₂Ph), 5.00 (2H, t, J=9.7 Hz, H-4), 5.02 (1H, d, J=11.3 Hz, 1×OCH₂Ph), 5.44 (1H, d, J_{1,2}=4.1 Hz, H-1), 5.61 (1H, adt, J_{3,F}=12.3 Hz, J_{3,4}=J_{2,3}=9.5 Hz, H-3), 6.87-7.54 (20H, m, Ar—H), 7.30; ¹³C NMR (126 MHz, CHLOROFORM-d) δ ppm 20.6, 20.6, 20.8 (3×OCOCH₃), 22.7 (C-1′), 61.63 (C-6), 67.0 (C-5) 68.0 (1C, d, J_{C-4,F}=7 Hz, C-4,), 68.7 (C-7′), 71.1 (1C, d, J_{C-3,F}=18.9 Hz, C-3), 71.1 (C-6′), 72.6 (OCH₂Ph), 73.1 (OCH₂Ph), 74.4 (OCH₂Ph or C-5′), 74.5 (OCH₂Ph or C-5′), 75.5 (OCH₂Ph), 80.1 (C-4′), 80.6 (C-3′) 87.5 (1C, d, J_{C-2,F} 194 Hz, C-2), 88.7 (1C, d, J_{C-1,F}=21 Hz, C-1), 102.0 (C-2′), 127.5, 127.5, 127.6, 127.7, 127.7, 128.0, 128.2, 128.3, 128.4 (4×OCH₂Ph), 138.1, 138.4, 138.7, 138.7 (4×1C, 4×OCH₂Ph), 169.7, 170.1, 170.5 (3×C═O); ¹⁹F NMR (1H) (377 MHz, CHLOROFORM-d) δ ppm −197.6; IR (thin film): ν=2922, 2852, 2408, 1747 (C═O), 1496, 1453, 1367, 1220, 1055, 772 cm⁻¹; MS m/z (ESI) 882.3 (NH₄); HRMS (ESI) calcd. for C₄₇H₅₃FO₁₃ (M+Na) 867.3365. Found: 867.3362.

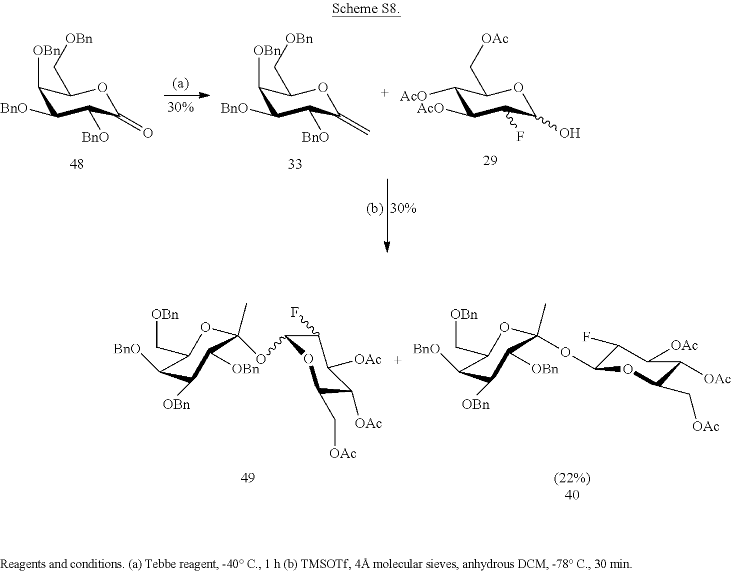

### 2,3,4,6-Tetra-O-benzyl-1-deoxy-α-D-galacto-hept-2-ulopyranosyl-(2→1)-3,4,6-tri-O-acetyl-2-deoxy-2-fluoro-β-D-glucopyranoside (40)

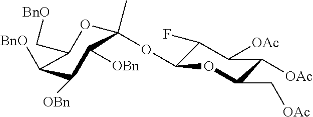

Following the procedure in Li, 2001¹³. A solution of 33 (168 mg, 0.31 mmol, 1 eq), and a 29 (178 mg, 0.58 mmol, 1.6 eq) and molecular sieves 4A (ca. 100 mg) in dry CH₂Cl₂ (5 mL) was stirred under nitrogen atmosphere at room temperature for 30 min. The solution was cooled to −78° C. and TMSOTf (5 μL, 0.027 mmol, 0.05 eq.) was added. The reaction mixture was stirred at −78° C. for 30 minutes. The reaction was monitored by TLC (2.5:1 petrol/ethyl acetate) and upon completion, the appearance of one broad spot (R_{f} 0.3) and disappearance of starting glycal (R_{f} 0.8) was detected. Reaction was quenched with triethylamine (20 μl) and passed through Celite® and concentrated. The residue was applied on a silica gel column chromatography (2:1 petrol/ethyl acetate) to afford the products as a 1:2 mixture of the α,α and α,β and gluco and manno sugars (75 mg, 30%). From these only the titled compound could be isolated to purity as a clear oil (57 mg, 22%). [α]_{D}²⁴+18.7 (c=1.0 in CHCl₃); ¹H NMR (500 MHz, CHLOROFORM-d) δ ppm 1.41 (3H, s, C-1′), 2.00 (6H, s, 2×OCOCH₃) 2.05 (3H, s, 1×OCOCH₃), 2.08 (3H, s, 1×OCOCH₃), 3.41 (1H, dt, J_{4,5}=10.1 Hz, J_{5,6a}=3.2 Hz, J_{5,6b}=3.2 Hz H-5), 3.45 (1H, dd, J_{7a,7b}=5.9 Hz, J_{6,71})=3.1 Hz, H-7_{b}′), 3.50-3.55 (1H, m, H-7ₐ′), 3.90 (1H, d, J_{3,4}=9.8 Hz, H-3′), 3.96 (2H, dd, J=5.9 Hz, J=3.0 Hz, H-6ₐ, H-6_{b}), 3.99 (1H, at, J_{4,5}=J_{5,6}=1.0 Hz, H-5′), 4.02 (1H, dd, J_{3,4}=9.8 Hz, H-4′), 4.27-4.35 (1H, m, H-2, H-6′), 4.41 (1 H, d, J=9.8 Hz, 1×OCH₂Ph), 4.45 (1H, d, J=11.3 Hz, 1×OCH₂Ph), 4.63 (1H, d, J=11.7 Hz, 1×OCH₂Ph), 4.72 (1H, d, J=11.3 Hz, 1×OCH₂Ph), 4.76 (2H, s, 2×OCH₂Ph), 4.93-4.97 (3H, m, H-1, 2×OCH₂Ph), 4.99 (1H, dd, J_{4,5}=7.2 Hz J_{3,4}=4.9 Hz, H-4), 5.25 (1H, dt, J_{3,F}=14.1 Hz, J_{2,3}=J_{3,4}=9.2 Hz. H-3), 7.19-7.44 (20 H, m); ¹³C NMR (126 MHz, CHLOROFORM-d) δ ppm 20.4, 20.5, 20.6 (3×OCOCH₃), 22.3 (C-1′), 61.4 (C-6), 67.9 (1C, d, J_{C-4,F}=7.6 Hz, C-4) 69.9 (C-7′), 71.8 (C-6′), 71.9 (C-5), 72.8 (OCH₂Ph), 73.3 (1C, d, J_{C-3,F}=20.1 Hz, C-3), 73.7 (OCH₂Ph), 74.3 (C-5′), 74.6 (OCH₂Ph), 75.6 (OCH₂Ph), 80.1 (C-3′), 80.4 (C-4′) 88.8 (C-2,d, J_{C-2,F}=190.8 Hz), 93.6 (C-1, d, J_{C-1,F}=22.9 Hz), 103.1 (C′-2), 127.4, 127.5, 127.5, 127.6, 127.7, 128.1, 128.1, 128.2, 128.2, 128.3, 128.4 (4×OCH₂Ph) 138.0, 138.3, 138.5, 138.6 (4×1C, 4×OCH₂Ph), 170.1, 170.5, 171.1 (3×C═O); ¹⁹F NMR (377 MHz, CHLOROFORM-d) δ ppm −198.73; IR (thin film): ν=3063, 3030, 2923, 2857, 1752 (C═O), 1604, 1548, 1496, 1454, 1367, 1230, 1212, 1139, 1100, 1054, 919, 808, 736 cm⁻¹; MS m/z (ESI⁺) 862.3 (M+NH₄⁺); HRMS (ESI) calcd. for C₄₇H₅₃O₁₃ (M+Na⁺) 867.3365. Found: 867.3383.

### 1-Deoxy-α-D-galacto-hept-2-ulopyranosyl-(2→1)-2-deoxy-2-fluoro-α-D-glucopyranoside (5)

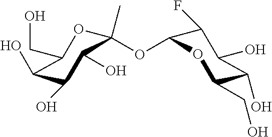

39 (42 mg, 0.05 mmol, 1 eq) was dissolved in 20 mL methanol with NaOMe (31 mg, 0.6 mmol, 12 eq) and stirred at room temperature for 3 hours until complete disappearance of starting material (R_{f} 0.55) and appearance of a new spot (R_{f} 0.05) was observed by TLC (1:1 petrol/ethyl acetate) and deacetylated sugar was detected by ESI⁺ m/z (M+Na): 741.3. Reaction was neutralized with DOWEX 50WX8 (H⁺ form) ion exchange resin and concentrated in vacuo. The resulting clear oil was re-dissolved in methanol (10 mL) and solution was degassed with nitrogen. To this was added basic alumina (31.0 mg) and 20% Pd(OH)₂/C (73 mg) and reaction was stirred under hydrogen atmosphere (balloon) at room temperature. After 96 h. reaction was filtered through filter paper and filtrate was evaporated. Further purification was obtained utilizing Isolute SPE C18 cartridge to give the desired, fully deprotected sugar, as a clear oil (20 mg, 100%).

[α]_{D}²⁵+122.8 (0.42 in MeOH); ¹H NMR (500 MHz, DEUTERIUM OXIDE) δ ppm 1.47 (3H, s, C-1′), 3.39 (1H, at, J_{3,4}=J_{4,5}=9.5 Hz, H-4), 3.52 (1H, d, J_{3,4}=10.4 Hz, H-3′), 3.61 (1H, dd, J_{7a,7b}=12.0 Hz, J_{6,7a}=5.4 Hz, H-7b′), 3.64 (1H, dd, J_{7a,7b}=12.0 Hz, J_{6,7a}=7.3 Hz, H-7° ′), 3.66 (1H, dd, J_{6a,6b}=12.6 Hz, J_{5,6a}=5.4 Hz, H-6a), 3.76 (1H, dd, J_{6a,6b}=12.3 Hz, J_{5,6b}=2.2 Hz, H-6b), 3.79 (1H, dd, J_{4,5}=10.1 Hz, J_{5,6a}=4.8 Hz, J_{5,6b}=2.6 Hz, H-5), 3.83 (1H, dd, J_{3,4}=10.1, J_{4,5}=3.2 Hz, H-4′), 3.90 (1H, dd, J_{4,5}=3.1 Hz, J_{5,6}=1.0 Hz, H-5′), 3.97 (1H, ddd, J_{6,7a}=7.1, J_{6,7b}=5.5 Hz, J_{5,6}=1.0 Hz, H-6′), 4.02 (1H, dt, J_{3,F}=13.0 Hz, J_{3,4}=J_{2,3}=9.4 Hz, H-3), 4.37 (1 H, m, J_{2,F}=49.5 Hz, J_{2,3}=9.5 Hz, J_{1,2}=3.8 Hz, H-2), 5.46 (1H, d, J_{1,2}=3.8 Hz, H-1); ¹³C NMR (126 MHz, DEUTERIUM OXIDE) δ ppm 22.44 (C-1′), 60.3 (C-6), 61.3 (C-7′), 69.3 (1C, d, J_{C-4,F}=7.5 Hz, C-4), 69.3 (C-5′), 69.6 (C-4′), 71.2 (1C, d, J_{C-3,F}=17.6 Hz, C-3), 71.6 (1C, J_{C-5,F}=6.3 Hz, C-5), 71.8 (C-6′), 73.2 (C-3′), 88.8 (1C, d, J_{C-1,F}=21.4 Hz, C-1), 89.9 (1C, d, J_{C-2,F}=189 Hz, C-2), 101.34 (C-2′); ¹⁹F NMR (1H) (377 MHz, DEUTERIUM OXIDE) δ ppm −197.5 (dt, J_{2,F}=51.6, J_{1,F}=J_{3,F}=14.9 Hz); MS m/z (ESI⁺) 381.2 (M+Na⁺); HRMS (ESI⁺) calcd. for C₁₃H₂₃FO₁₀ (M+Na⁺): 381.1173. Found: 381.1165.

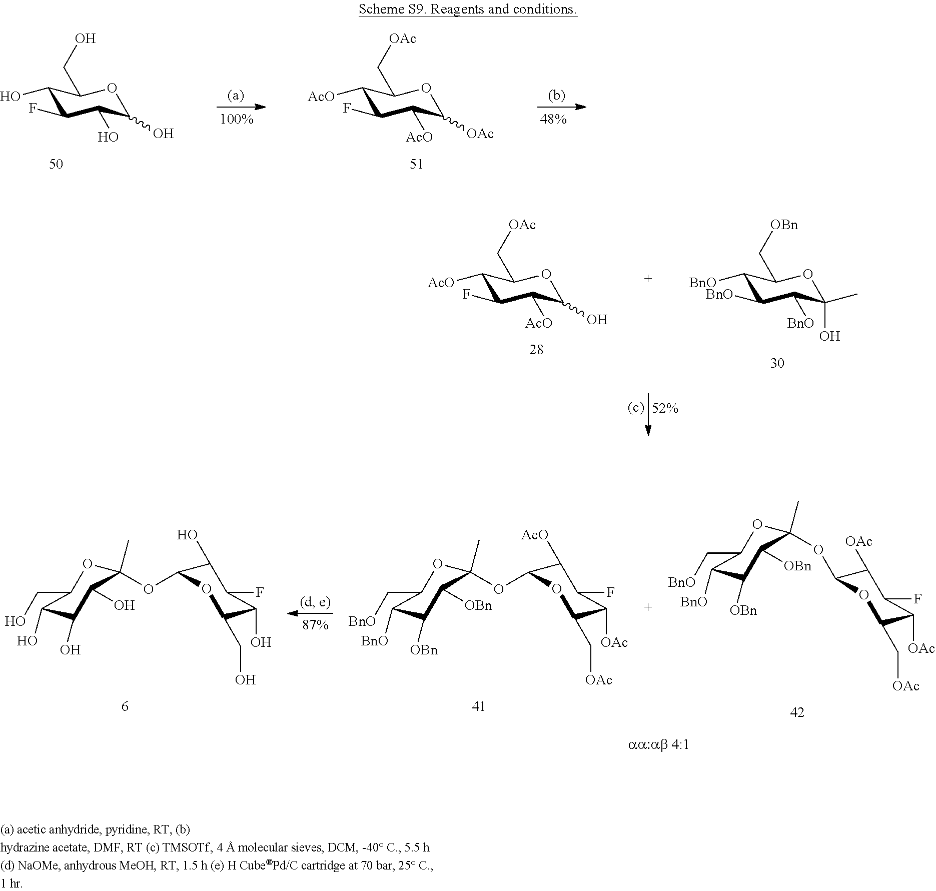

### 3,4,6,7-Tetra-O-benzyl-1-deoxy-α-D-gluco-hept-2-ulopyranosyl-(2→1)-2,4,6-tri-O-acetyl-3-deoxy-3-fluoro-α-D-glucopyranoside (41)

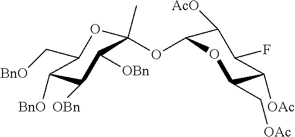

30 (116 mg, 0.21 mmol, 1.16 eq) and 28 (56 mg, 0.18 mmol, 1 eq) were dried under reduced pressure for one hour and then dissolved in anhydrous DCM (8 mL) and added to activated molecular sieves (ca 100 mg). Sugars were stirred with sieves at RT for 1 hour and were then cooled to −40° C. TMSOTf (10 μl, 0.054 mmol, 0.3 eq) was added and 1 hour later additional TMSOTf (10 μl, 0.054 mmol, 0.3 eq) was added to the reaction. Reaction was stirred for 5.5 h, upon which time conversion was detected by TLC (2:1 petrol/ethyl acetate) with conversion to product α,α (R_{f} 0.5) and α,β (R_{f} 0.48) and disappearance of starting sugars (Rf 0.6) and (Rf 0.05). Reaction was then quenched with 1 drop triethylamine, filtered through Celite® to remove molecular sieves, concentrated under reduced pressure and purified by column chromatography (2.5:1 petrol/ethyl acetate). The desired product was obtained as a clear oil (79 mg, 52%) α,α (63 mg) and α,β (16 mg) (4:1α,α:α,β) as well as recovered 28 (10 mg).

[α]_{D}²⁵=66.3 (c=1.0, CHCl₃); ¹H NMR (400 MHz, CHLOROFORM-d) δ ppm 1.47 (3H, s, C-1′), 2.02 (3H, s, 1×OCOCH₃), 2.04 (3H, s, 1×OCOCH₃), 2.13 (3 H, s, 1×OCOCH₃), 3.33 (1H, d, J_{3,4}=9.6 Hz, H-3′), 3.58-3.70 (3H, m, H-7a′,H-7b′, H-5′), 3.81 (1H, dd, J_{6a,6b}=12.4 Hz, J_{5,6b}=1.6 Hz, H-6b), 3.90 (1H, ddd, J_{5,6}=10.1 Hz, J_{6,7a}=4.0 Hz, J_{6,7b}=2.3 Hz, H-6′), 4.02 (1H, at, J_{3,4}=J_{4,5}=9.3 Hz, H-4′), 4.05 (1H, dd, J_{6a,66}=12.9 Hz, J_{5,6a}=4.5 Hz, H-6a), 4.25 (1H, ddd, J_{4,5}=10.2 Hz, J_{5,6a}=4.2 Hz, J_{5,6b}=2.0 Hz, H-5), 4.50 (1H, d, J=12.1 Hz, 1×OCH₂Ph), 4.58 (1H, d, J=9.3 Hz, 1×OCH₂Ph), 4.60 (1H, d, J=12.1 Hz, 1×OCH₂Ph), 4.64 (1H, d, J=11.4 Hz, 1×OCH₂Ph), 4.87 (1H, d, J=10.9 Hz, 1×OCH₂Ph), 4.90 (1H, dt, J_{3,F}=54.0 Hz, J_{2,3}=9.4 Hz, J_{3,4}=9.4 Hz, H-3), 4.92 (2H, m, 2×OCH₂Ph), 4.99 (1H, d, J=11.1 Hz, 1×OCH₂Ph), 5.08 (1H, ddd, J_{2,F}=11.4 Hz, J_{2,3}=9.9 Hz, J_{1,2}=3.5 Hz, H-2), 5.21 (1H, ddd, J_{4,F}=13.6 Hz, J_{4,5}=10.4 Hz, J_{3,4} 9.1 Hz, H-4), 5.39 (1H, at, J_{1,2}=3.4 Hz, H-1), 7.08-7.44 (20H, m, Ar—H); ¹³C NMR (126 MHz, CHLOROFORM-d) δ ppm 20.6, 20.6, 20.7 (3×OCOCH₃), 22.5 (C-1′), 61.5 (C-6), 67.1 (1C, d, J_{C-5,F}=6.3 Hz, C-5), 68.3 (1C, d, J_{C-4,F}=17.4 Hz, C-4), 68.5 (C-7′), 70.7 (1C, d, J_{C-2F}=16.4 Hz, C-2) 72.1 (C′-6), 73.5 (OCH₂Ph), 74.8 (OCH₂Ph), 75.5 (OCH₂Ph), 75.5 (OCH₂Ph), 78.2 (C-5′), 82.74 (C-4′), 84.5 (C-3′), 89.6 (1C=d, J_{C-1,F}=8.8 Hz C-1), 89.6 (1C, d, J_{C-3,F}=189 Hz, C-3), 101.4 (C-2′), 127.5, 127.6, 127.7, 127.7, 127.8, 127.9, 127.9, 128.2, 128.3, 128.4 (4×OCH₂Ph), 137.8, 138.2, 138.4, 138.6 (4×1C, 4×OCH₂Ph), 169.3, 169.7, 170.5 (3×C═O); ¹⁹F NMR (1H) (377 MHz, CHLOROFORM-d) δ ppm −199.9 (1F, s); IR (thin film): ν=3062.8, 3030.0, 2923.4, 2863.6 (C═CH), 1751 (C═O), 1496, 1453, 1367, 1219, 1131, 1089, 1067, 1038, 736; MS m/z (ESI⁺) 903.3 (M+MeCN+NH₄⁺); HRMS (ESI⁺) calcd. for C₄₇H₅₃FO₁₃ (M+NH₄¹) 867.3362 Found: 867.3338.

### 3,4,6,7-Tetra-O-benzyl-1-deoxy-α-D-gluco-hept-2-ulopyranosyl-(2→1)-2,4,6-tri-O-acetyl-3-deoxy-3-fluoro-β-D-glucopyranoside (42)

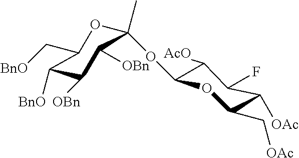

42 was isolated as the lower spot TLC (2:1 petrol/ethyl acetate) (R_{f} 0.45) of the reaction between 30 and 28 as a clear oil (16 mg, 10%).

[α]_{D}²⁵=12.4 (c=1.0, CHCl₃); ¹H NMR (500 MHz, CHLOROFORM-d) δ ppm 1.43 (3H, s, C-1′), 2.04-2.20 (9H, m, 3×OCOCH₃), 3.34 (1H, d, J_{3,4}=9.8 Hz, H-3′), 3.52 (1H, adt, J_{4,5}=9.9 Hz, J_{5,6a}=J_{5,6b}=3.3 Hz, H-5), 3.59 (1H, dd, J_{7a,7b}=10.7, J_{6,7b}=1.9 Hz, H-7b′), 3.69 (1H, at, J_{4,5}=J_{4,6}=9.5 Hz, H-5′), 3.70 (1H, dd, J_{7a,7b}=10.4 Hz, J_{6,7a}=3.8 Hz, H-7a′), 4.06 (2H, at, J_{3,4}=J_{4,5}=9.5 Hz, H-4′), 4.07 (2H, dd, J_{6a,6b}=12.1 Hz, J_{5,6b}=2.2 Hz, H-6b), 4.12 (2H, dd, J_{6a,6b}=12.3 Hz, J_{5,6a}=4.7 Hz, H-6a), 4.16 (1H, ddd, J_{5,6}=10.2 Hz, J_{6,7a}=3.5 Hz, J_{6,7b}=2.0 Hz, H-6′), 4.51 (1H, dt, J_{3,F}=53.0 Hz, J_{2,3}=9.1 Hz, J_{3,4}=9.1 Hz H-3), 4.47 (1H, d, J=12.0 Hz, 1×OCH₂Ph), 4.53 (1H, d, J=11.0 Hz, 1×OCH₂Ph), 4.59 (1H, d, J=12.3 Hz, 1×OCH₂Ph), 4.60 (1H, d, J=11.3 Hz, 1×OCH₂Ph), 4.81 (1H, d, J_{1,2}=7.9 Hz, H-1), 4.84 (1H, d, J=10.7 Hz, 1×OCH₂Ph), 4.86-4.90 (2H, m, 2×OCH₂Ph), 4.94 (1H, d, J=11.3 Hz, 1×OCH₂Ph), 5.14 (2H, m, H-4, H-2), 7.21-7.37 (20H, m, Ar—H); ¹³C NMR (126 MHz, CHLOROFORM-d) δ ppm 20.4, 20.5, 20.6 (3×OCOCH₃), 22.0 (C-1′), 61.9 (C-6), 68.4 (1C, d, J_{C-4,F}=12.6 Hz, C-4), 68.5 (C-7′) 70.9 (1C, d, J_{C-2,F}=18.9 Hz, C-2), 71.2 (1C, d, J_{C-5,F} 7.5 Hz C-5), 72.4 (H-6′), 73.4 (OCH₂Ph), 74.9 (OCH₂Ph), 75.5 (OCH₂Ph), 75.6 (OCH₂Ph), 78.1 (H-5′), 82.5 (C-4′), 84.5 (C-3′), 91.7 (1C, d, J_{C-2,F}=191.5 Hz, C-3), 94.2 (1C, d, J_{C-1,F}=12.6 Hz, C-1), 102.4 (C-2′), 127.5, 127.6, 127.6, 127.6, 127.8, 128.3, 128.3, 128.4, 128.4 (4×OCH₂Ph), 138.2, 138.2, 138.4, 138.6 (4×1C. 4×OCH₂Ph), 168.8, 169.15, 170.6 (3×C═O); IR (thin film): ν=3062, 3030, 2923, 2855 (C═CH), 1752 (C═O), 1496, 1453, 1368, 1218, 1151, 1126, 1063, 1043. 737; MS m/z (ESI⁺) 903.3 (M+MeCN+NH₄⁺); HRMS (ESI⁺) calcd. for C₄₇H₅₃FO₁₃ (M+NH₄⁺): 867.3362. Found: 867.3338.

### 1-Deoxy-α-D-gluco-hept-2-ulopyranosyl-(2→1)-3-fluoro-3-deoxy-α-D-glucopyranoside (6)

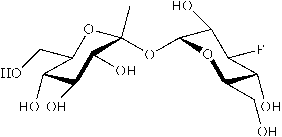

41 (42 mg, 0.049 mmol, 1 eq) was dissolved in anhydrous methanol (10 mL) to this was added sodium methoxide (20 mg, 0.37 mmol, 7.5 eq) and reaction was stirred under argon atmosphere for 1.5 hours until complete conversion to product was detected by TLC (2:1 petrol/ethyl acetate) (R_{f} 0.0) with disappearance of starting sugar (R_{f} 0.5). Reaction was neutralized with DOWEX, 50WX8 (H⁺ form) cation exchange resin. Resin was removed by filtration and filtrate was concentrated under reduced pressure and redissolved in ethanol (10 mL). Reaction mixture was circulated through a Thales Nano H Cube® Pd/C cartridge at 70 bar, 25° C. for 1 hour. Near complete deprotection was detected by TLC (1:1 methanol/ethyl acetate) (R_{f} 0.45). Reaction mixture was partitioned between water and DCM and aqueous layer was lyophilized. Further purification was obtained utilizing Isolute SPE C18 cartridge to yield the desired product (15.6 mg, 87%) as a white, amorphous solid.

[α]_{D}²⁵=108.2 (c=0.22, MeOH); ¹H NMR (500 MHz, DEUTERIUM OXIDE) δ ppm 1.44 (3H, s, C-1′), 3.23 (1H, d, J_{3,4}=9.8 Hz, C-3′), 3.34 (1H, at, J_{4,5}=J_{5,6}=9.6 Hz, C-5′), 3.62-3.75 (6H, m, H-6a, H-6b, H-7a, H-7b, H-4, H-4′), 3.79 (1H, ddd, J_{2,F}=16.4 Hz, J_{2,3}=9.0 Hz, J_{1,2}=3.6 Hz, H-2), 3.80 (3H, ddd, J_{4,5}=11.7 Hz, J_{5,6a}=6.3 Hz, J_{5,6b}=3.5 Hz, H-5′), 3.97 (2H, ddd, J_{5,6}=10.1 Hz, J_{6,7a}=5.4 Hz, J_{6,7b}=2.2 Hz, H-6′), 4.68 (1H, dt, J_{3,F}=55.0 Hz, J_{3,4}=9.1 Hz, J_{2,3}=9.1 Hz, H-3), 5.26 (2 H, at, J=3.6 Hz, H-1); ¹³C NMR (126 MHz, DEUTERIUM OXIDE) δ ppm 22.4 (C-1′), 60.1 (C-6), 60.5 (C-7′), 68.1 (1C, d, J_{C-4,F}=16.4 Hz, C-4), 69.8 (C-5′), 69.9 (1C, d, J_{C-2,F}=16.4 Hz, C-2), 71.3 (1C, d, J_{C-5,F}=7.5 Hz, C-5), 72.3 (C-6′) 72.8 (C-4′), 76.3 (C-3′), 91.7 (1C, d, J_{C-3,F}=11.3 Hz, C-1), 94.5 (1C, d, J_{C-3,F}=180.1 Hz, C-3), 101.1 (C-2′); ¹⁹F NMR (377 MHz, DEUTERIUM OXIDE) δ ppm −199.2 (83 F, dtt, J_{3,F}=55.1, J_{2,F}=J_{4,F}=14.9 Hz, J_{1,F}=J_{5,F}=3.4 Hz); MS m/z (ESI⁻) 357.1 (M−H); HRMS (ESI⁺) calcd. for C₁₃H₂₃FO₁₀ (M−H+): 357.1205. Found: 357.1197.

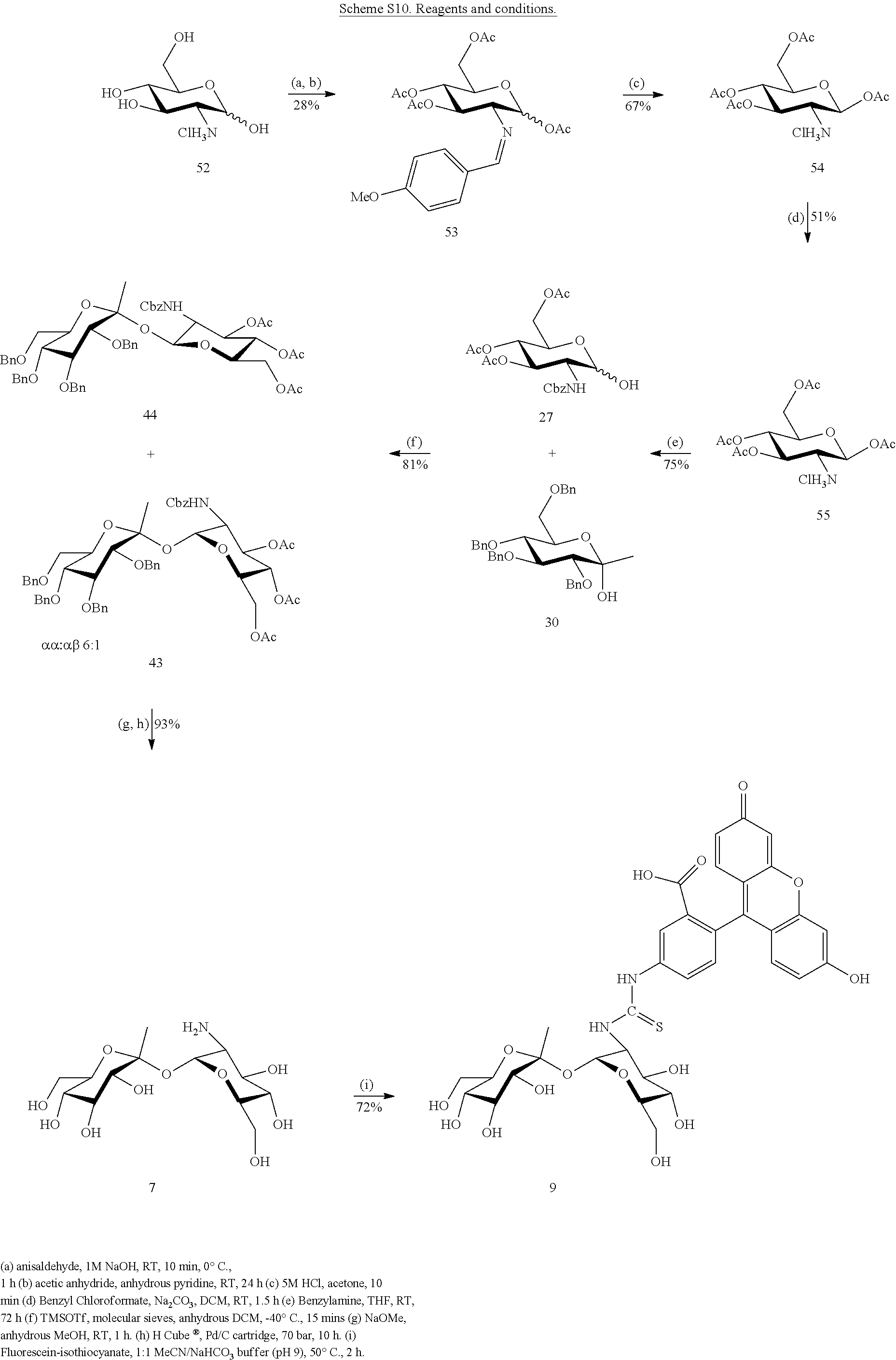

### 3,4,6,7-Tetra-O-benzyl-1-deoxy-α-D-gluco-hept-2-ulopyranosyl-(2→1)-3,4,6-tetra-O-acetyl-2-deoxy-2-benzylcarbamate-α-D-glucopyranoside (43)

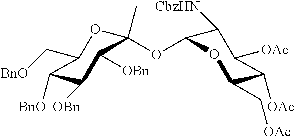

30 (173.8 g, 0.31 mmol, 1 eq) and 27 (168.0 mg, 0.38 mmol, 1.2 eq) were dried under reduced pressure for 1 hour and then dissolved in anhydrous DCM (10 mL) and added to a dry flask in the presence of molecular sieves (ca. 100 mg). Mixture was stirred with molecular sieves for 30 minutes at room temperature and then was cooled to −40° C. To this was added TMSOTf (10 μl, 0.054 mmol. 0.15 eq) at −40° C. in the under an Ar atmosphere. The resulting mixture was stirred for 15 minutes until TLC (1.5:1 petrol/ethyl acetate) revealed the production of two new products (R_{f} 0.6 and R_{f} 0.35) and consumption of starting material (R_{f} 0.35 and R_{f} 0.05). The reaction was then quenched by the addition triethylamine (0.1 mL), filtered through Celite® and concentrated. The crude product was purified by column chromatography (5:2 petrol/ethyl acetate) to yield the desired compound α,α (214 mg) as well as the α,β (32 mg) for a net yield (81%, 6:1α,α: α,β) as well as recovered 30 (14.5 mg, 8%).

[α]_{D}²⁵=67.3 (c=0.94, CHCl₃). ¹H NMR (500 MHz, CHLOROFORM-d) δ ppm 1.48 (3H, s, C-1′), 1.96 (3H, s, 1×OCOCH₃), 2.01 (3H, s, 1×OCOCH₃), 2.04 (3H, s, 1×OCOCH₃), 3.33 (1H, d, J_{3,4}=9.4 Hz, C-3′), 3.43 (1H, dd, J_{7a,7b}=10.9 Hz, J_{6,7b}=1.42 Hz, H-7b′), 3.53 (1H, dd, J_{7a,7b}=11.03 Hz, J_{6,7a}=2.8 Hz, H-7a′), 3.68-3.79 (3H, m, H-5′, H-6b, H-6′), 3.97 (1H, at, J_{3,4}=J_{4,5}=9.1 Hz, H-4′), 4.04 (2H, dd, J_{5,6a}=12.6 Hz, J_{6a,6b}=4.41 Hz, H-6a), 4.05 (1H, dd, J_{2,3}=10.7 Hz, J_{1,2}=3.8 Hz, H-2), 4.26 (1H, ddd, J_{4,5}=10.3 Hz, J_{5,6a} 4.5 Hz, J_{5,6b}=2.21 Hz, H-5), 4.38 (1 H, d, J=12.3 Hz, 1×OCH₂Ph), 4.53 (1H, d, J=12.3 Hz, 1×OCH₂Ph), 4.56 (1H, d, J=11.0 Hz, 1×OCH₂Ph), 4.61 (1H, d, J=11.4 Hz, 1×OCH₂Ph), 4.82 (1H, d, J=11.0 Hz, 1×OCH₂Ph), 4.91 (1H, d, J=11.9 Hz, 1×OCH₂Ph Cbz), 4.93 (1H, d, J=10.9 Hz, 1×OCH₂Ph), 4.95 (1H, d, J=12.6 Hz, 1×OCH₂Ph), 4.97 (1H, d, J=11.4 Hz, 1×OCH₂Ph Cbz), 5.05 (1H, d, J=12.0 Hz, 1×OCH₂Ph), 5.08 (1H, at, J_{3,4}=J_{4,5=9.1} Hz, H-4), 5.30 (1H, dd, J_{2,3}=10.7 Hz, J_{3,4}=9.4 Hz, H-3), 5.36 (1H, d, J_{1,2}=3.5 Hz, H-1), 6.98-7.41 (25H, m, Ar—H); ¹³C NMR (126 MHz, CHLOROFORM-d) δ ppm 20.6, 20.7 (3×OCOCH₃), 23.1 (C-1′), 54.6 (C-2), 61.7 (C-6), 67.0 (OCH₂Ph Cbz), 67.2 (C-5), 68.1 (C-7′), 68.3 (C-4), 70.9 (C-3), 72.3 (C-5′), 73.4 (OCH₂Ph), 74.9 (OCH₂Ph), 75.5 (OCH₂Ph), 75.7 (OCH₂Ph), 77.9 (C-6′), 82.8 (C-4′), 84.7 (C-3′), 90.7 (C-1), 101.6 (C-2′), 127.6, 127.6, 127.6, 127.8, 127.8, 128.1, 128.3, 128.4, 128.4 (5×OCH₂Ph), 136.0, 138.1, 138.2, 138.3, 138.5 (5×1C. 5×OCH₂Ph), 155.6 (1×C═O Cbz), 169.4, 170.6, 171.4 (3×CO)═; IR (thin film): ν=3345.5, (br. Amide), 3088.8, 2924.1, 2863.6 (C═CH), 1745.4 (C═O), 1720.0 (C═O), 1564.1, 1529.7, 1453.7, 1365.4, 1230.0, 1153.1, 1028.11, 736.6; MS m/z (ESI⁺) 993.4 (M+NH₄⁺); HRMS (ESI⁺) calcd. for C₅₅H₆₁N₁O₁₅ (M+NH₄⁺): 993.4379. Found: 993.4377.

### 3,4,6,7-Tetra-O-benzyl-1-deoxy-α-D-gluco-hept-2-ulopyranosyl-(2→1)-3,4,6-tri-O-acetyl-2-deoxy-2-benzylcarbamate-β-D-glucopyranoside (44)

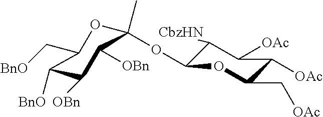

44 was isolated at the lower spot (R_{f} 0.15) of the reaction between 30 and 27 (212 mg, 7%). [α]_{D}²⁵=37.7 (c=1.0, CHCl₃). ¹H NMR (500 MHz, CHLOROFORM-d) δ ppm 1.36 (3H, s, C-1′), 1.94 (3H, s, 1×OCOCH₃), 2.00 (3H, s, 1×OCOCH₃), 2.01 (3H, s, 1×OCOCH₃), 3.35 (1H, d, J_{3,4}=9.5 Hz, H-3′), 3.46 (1H, br. s., H-2), 3.62 (2H, adt, J_{4,5}=10.7 Hz, J_{5,6a}=J_{5,6b}=1.1 Hz, H-5, H-7b′), 3.71 (2H, at, J_{4,5}=J_{45,6}=9.8 Hz, H-5′), 3.74 (1H, dd, J_{7a,7b}=10.7 Hz, J_{6,7a}=3.2 Hz, H-7a′), 3.99 (1H, at, J_{3,4}=J_{4,5}=9.5 Hz, H-4′), 4.05 (1H, dd, J_{6a,6b}=12.3 Hz, J_{5,6b}=2.2 Hz, H-6b), 4.12 (1H, dd, J_{6a,6b}=12.0 Hz, J_{5,6a}=4.7 Hz, H-6a), 4.17 (1H, ddd, J_{5,6}=10.7 Hz, J_{6,7a}=3.3 Hz, J_{6,7b}=2.0 Hz, H-6′), 4.49 (1H, d, J=12.3 Hz, 1×OCH₂Ph), 4.56 (1 H, d, J=10.7 Hz, 1×OCH₂Ph), 4.60 (1H, d, J=10.7 Hz, 1×OCH₂Ph), 4.61 (1H, d, J=12.3 Hz, 1×OCH₂Ph), 4.77-4.85 (3H, m, 1×OCH₂Ph Cbz, 2×OCH₂Ph), 4.88 (2H, d, J=11.0 Hz, 1×OCH₂Ph), 4.95 (1H, at, J_{3,4}=J_{4,5}=9.8 Hz, H-4), 5.06-5.18 (2H, m, H-1, 1×OCH₂Ph Cbz), 5.46 (1H, at, J_{2,3}=J_{3,4}=9.6 Hz H-3), 7.14-7.37 (35 H, m, Ar—H); ¹³C NMR (126 MHz, CHLOROFORM-d) δ ppm 20.6, 20.7, 21.0 (3×OCOCH₃), 22.1 (CH₃), 56.5 (C-2), 62.4 (C-6), 66.9 (OCH₂Ph Cbz), 68.4 (C-4), 68.8 (C-7′), 71.85 (C-3), 72.0 (C-5), 72.3 (C-6′), 73.5 (OCH₂Ph), 74.9 (OCH₂Ph), 75.4 (OCH₂Ph), 75.8 (OCH₂Ph), 78.2 (C-5′), 82.8 (C-4′), 84.7 (C-3′), 94.6 (C-1), 102.3 (C-2′), 127.5, 127.6, 127.6, 127.7, 127.8, 127.9, 128.0, 128.1, 128.3, 128.4, 128.5 (5×OCH₂Ph), 136.2, 137.9, 138.2, 138.3, 138.7 (5×1C. 5×OCH₂Ph), 155.5 (1×C═O Cbz), 169.6, 170.4, 170.6 (3×C═O); IR (thin film): ν=3356.2 (Br. Amide), 3031.3, 2939.0 (C═CH), 1747.9 (C═O), 1586.5, 1454.1, 1367.1, 1230.5, 1043.1, 739.3; MS m/z (ESI⁺) 993.4 (M+NH₄⁺); HRMS (ESI⁺) calcd. for C₅₅H₆₁NO₁₅ (M+Na⁺): 998.3939. Found: 998.3905

### 1-Deoxy-α-D-gluco-hept-2-ulopyranosyl-(2→1)-2-amino-2-deoxy-α-D-glucopyranoside (7)

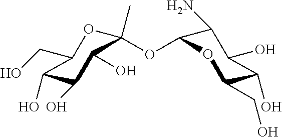

43 (578 mg, 0.59 mmol, 1 eq) was dissolved in anhydrous methanol (15 mL). To this was added sodium methoxide (25 mg, 0.46 mmol, 0.94 eq) and reaction was stirred for 1 hour, upon which time full conversion to product was detected by TLC (ethyl acetate) (R_{f} 0.75) and disappearance of starting sugar (R_{f} 1). Reaction was neutralized with DOWEX 50WX8 (H⁺ form) cation exchange resin (ca 50 mg). DOWEX was removed by filtration and reaction was concentrated under reduced pressure to yield the deacetylated product (507 mg, 100%). This product was split into two portions and each portion was dissolved in 20 mL (1:1 trifluoroethanol/water) with formic acid (50 μl) and cycled through the Thales Nano H Cube® over a Pd/C cartridge at 70 bar for 10 hours. Upon completion product was detected by TLC (1:2:2 water/isopropanol/ethyl acetate) (R_{f} 0.05) with complete disappearance of starting sugar (Rf 1). Reactions were concentrated under reduced pressure, redissolved in water and lyophilized to yield the desired product as a brownish, amorphous solid as the formate salt. Amine was further purified by a 10 ml column of DOWEX 50WX8 (H⁺ form) cation exchange resin. Amine was loaded onto resin, washed with water (20 ml), 0.1% NH₄OH (20 ml) and eluted with 5% NH₄OH (20 ml) to yield the desired product as a white amorphous solid. (221 mg, 93%).

[α]_{D}²⁵=^{83.8} (c=0.21, MeOH); ¹H NMR (500 MHz, DEUTERIUM OXIDE) δ ppm 1.48 (3H, s, C-1′), 2.74 (1H, d, J=10.4 Hz, H-2), 3.23 (1H, d, J_{3,4}=9.7 Hz, H-3′), 3.35 (2H, atd, J=9.6, 5.7 Hz, H-5, H-6′), 3.60-3.83 (8H, m, H-7a′, H-7b′, H-6a, H-6b, H-4′, H-5′, H-3, H-4), 5.24 (1H, d, J₁₂=3.2 Hz, H-1); ¹³C NMR (126 MHz, DEUTERIUM OXIDE) δ ppm 22.9 (C-1′), 55.5 (C-2), 60.5 (C-6), 60.6 (C-7′), 69.7 (C-5 or C-6′), 69.8 (C-5 or C-6′), 72.1 (C-4), 72.6 (C-4′ or C-5′), 72.7 (C-4′ or C-5′), 73.5 (C-3) 76.4 (C-3′), 91.95 (C-1), 101.3 (C-2′); MS m/z (ESI) 378.1 (M+Na⁺); HRMS (ESI⁺) calcd. for C₁₃H₂₅NO₁₀ (M+Na) 378.1371 Found: 378.1361.

### 1-Deoxy-α-D-gluco-hept-2-ulopyranosyl-(2→1)-2-N-fluorescein-2-deoxy-α-D-glucopyranoside (9)

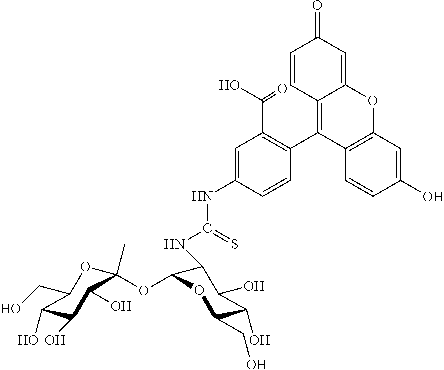

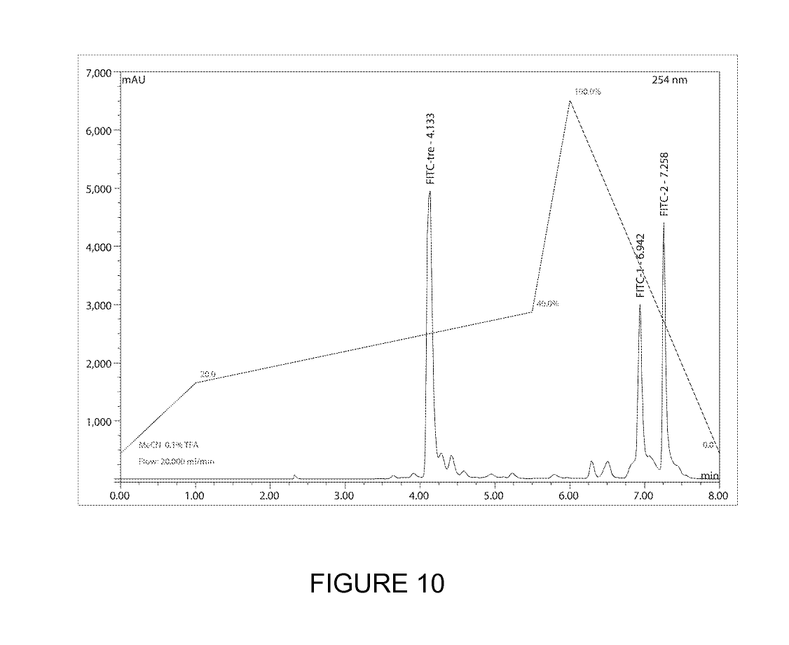

7 (11 mg, 0.03 mmol, 1 eq) and fluorescein isothiocyanate (17 mg, 0.044 mmol, 1.4 eq) were dissolved in 75 mM NaHCO₃ buffer at pH 9 (1 mL) with acetonitrile (0.5 mL). Reaction was heated to 50° C. for 2 upon which time product (R_{f} 0.3) was detected by TLC (1:2:2 water/isopropanol/ethyl acetate) with near complete disappearance of florescein starting material (R_{f} 0.8) and starting amine (R_{f} 0.05). Reaction mixture was purified by HPLC with a Phenomenex Synergi Hydro C18 column (150 mm×21.2 mm, 4 μm) and a acetonitrile gradient with 1% aqueous TFA, as shown in FIG. 10. Lyophilization yielded the desired product as a yellow solid (16 mg, 72%). [α]_{D}²⁵=72.2 (c=0.18, MeOH); ¹H NMR (500 MHz, DEUTERIUM OXIDE) δ ppm 1.48 (3H, s, C-1′), 3.26 (1H, d, J_{3,4}=9.8 Hz, H-3′), 3.49 (3H, m, H4′, H-5′, H-4), 3.74 (6H, m, H5, H-6′, H-6a, H-6b, H-7a′, H-7b′), 4.00 (1H, at, J_{2,3}=J_{3,4}=9.8 Hz, H-3), 4.41 (1H, dd, J_{2,3}=10.9 Hz, J_{1,2}=2.9 Hz, H-2), 5.59 (1H, d, J_{1,2}=3.6 Hz, H-1), 6.84 (4H, t, J=0.9 Hz), 7.12-7.18 (3H, m), 7.68 (1H, dt, J=7.7, J=0.9 Hz), 8.00 (1H, d, J=1.0 Hz); ¹³C NMR (126 MHz, DEUTERIUM OXIDE) δ ppm 22.79 (C-1′), 58.8 (C-2), 60.0 (C-7′), 60.56 (C-6), 68.9 (C-4), 70.4 (C-3, C-4′), 71.9 (C-6′), 72.8 (C-5), 72.8 (C-5′), 76.3 (C-3′), 89.6 (C-1), 101.5 (C-2′), 102.5 (C-isothiourea), 113.9, 115.1, 117.1, 117.4, 128.3, 130.2, 131.4, 140.4, 156.2, 162.8, 163.1, 166.0, 170.2, 181.6; MS m/z (ESI⁺) 743.2 (M−H); HRMS (ESI⁺) calcd. for C₃₄H₃₆NO₁₅S (M−H) 743.1758 Found: 743.1754.

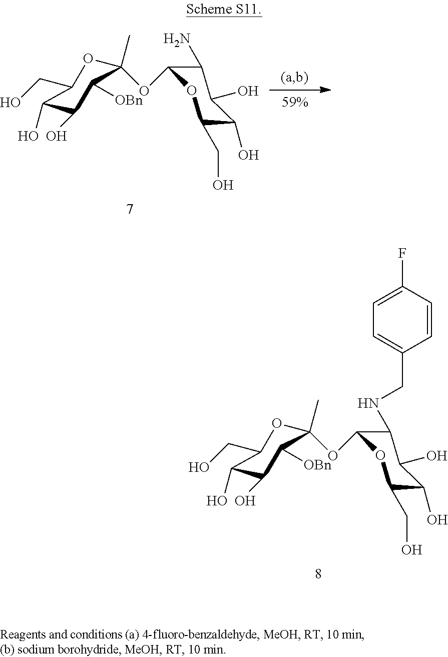

### 1-Deoxy-α-D-gluco-hept-2-ulopyranosyl-(2→1)-2-N-parafluorobenzoyl-2-deoxy-α-D-glucopyranoside trifluoroacetic acid salt (8)

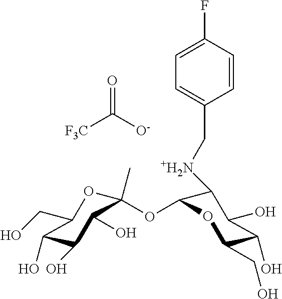

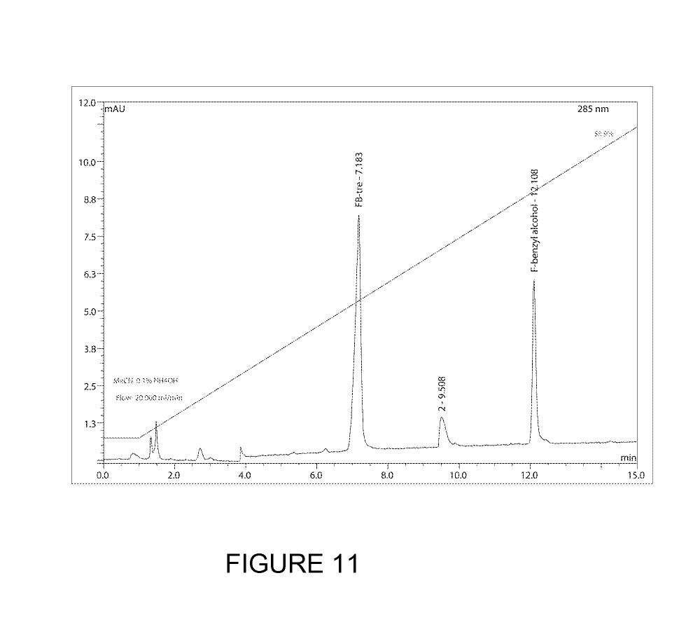

7 (11 mg, 0.03 mmol, 1 eq) was dissolved in methanol (1 mL). 4-F-benzaldehyde (9.5 μl, 0.06 mmol, 2 eq) was added to the reaction. After 10 minutes, complete conversion to imine was detected by TLC (2:1 ethyl acetate/methanol) product (R_{f} 0.4). To this was added NaBH₄ (15.2 mg, 0.4 mmol, 13.1 eq) and reaction was allowed to stir for a further 10 minutes, upon which time reductive amination was detected by ESI mass spec as well as TLC (1:2:2 water/isopropanol/ethyl acetate) product (R_{f} 0.3). Upon conversion, reaction was purified by HPLC with a HPLC with a Phenomenex Synergi Hydro C18 column (150 mm×21.2 mm, 4 μm) and an MeCN/H₂O gradient (5%/min) with 0.1% NH₄OH, as shown in FIG. 11. Lyophilization yielded the desired compound as a off-white amorphous solid as the TFA salt. (8.4 mg, 59%).

[α]_{D}²⁵=32.4 (c=0.29, MeOH); ¹H NMR (500 MHz, DEUTERIUM OXIDE) δ ppm 1.47 (3H, s, C-1′), 2.63 (1H, dt, J_{2,3}=10.4 Hz, J_{1,2}=2.8 Hz, J_{2, NH}=2.8 Hz, H-2), 3.22 (1H, d, J_{3,4}=9.8 Hz, H-3′), 3.29 (1H, at, J_{4,5}=J_{5,6}=9.5 Hz, H-5′), 3.36 (1H, at, J_{3,4}=J_{4,5}=9.6 Hz, H-4), 3.59 (1H, dd, J_{7a,7b}=12.3 Hz, J_{6,7a}=5.0 Hz, H-7a′), 3.65 (1H, dd, J_{6a,6b}=12.3 Hz, J_{5,6a}=5.0 Hz, H-6a), 3.67 (1H, dd, J_{6a,6b}=12.6 Hz, J_{5,6b}=2.5 Hz, H-6b), 3.71-3.77 (3H, m, H-7b′, H-6′, H-4′), 3.79-3.86 (3H, m, H-3, 2×NCH₂Ph), 3.86 (1H, ddd, J_{4,5}=9.8 Hz, J_{5,6a}=4.4 Hz, J_{5,6b}=2.2 Hz, H-5), 5.36 (1H, d, J_{1,2}=2.8 Hz, H-1), 7.05 (2H, at, J_{ortho,meta}=J_{H,F}=9.0 Hz, Ar—Hₒᵣₜₕₒ), 7.30 (2H, dd, J_{ortho,meta}=8.4 Hz, J_{H,F}=5.8 Hz, Ar—Hₘₑₜₐ); ¹³C NMR (126 MHz, DEUTERIUM OXIDE) δ ppm 22.5 (C-1′), 50.0 (NCH₂Ph-F), 60.3 (C-2), 60.6 (C-6), 60.7 (C-7′), 69.6 (C-4), 70.4 (C-5′), 71.8 (C-4′), 72.4 (C-3), 72.6 (C-6′), 72.7 (C-5), 76.5 (C-3′), 90.5 (C-1), 101.4 (C-2′), 115.1, 115.2, 115.3, 117.3, 117.5, 119.8, 129.7, 129.8 (Ar—C), 162.8, 162.9 (C═O, TFA); ¹⁹F NMR (1H) (377 MHz, CHLOROFORM-d) δ ppm −75.6 (TFA), −116.3 (F-Benzyl); MS m/z (ESI⁺) 486.2 (M+Na⁺); HRMS (ESI⁺) calcd. for C₂₀H₃₀FNO₁₀ (M+Na⁺): 486.1751. Found: 486.1770.

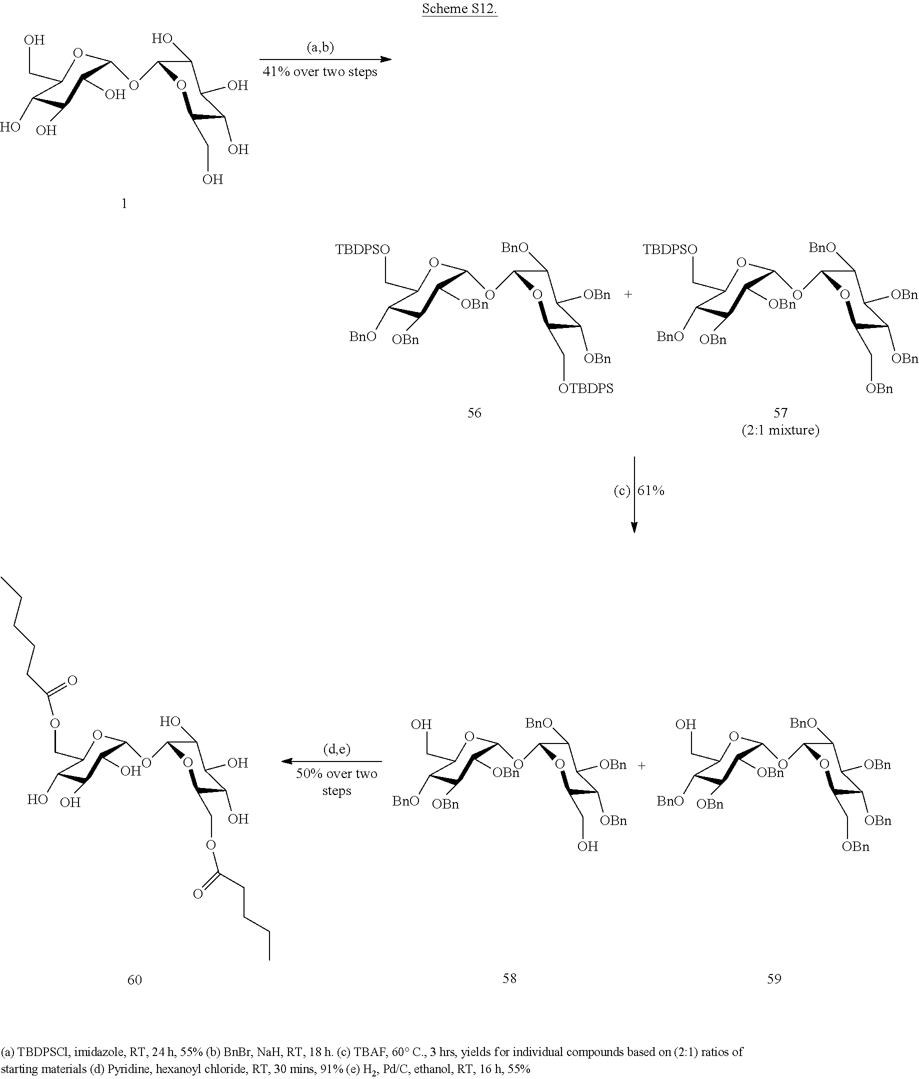

### 6,6′-O-Di-tertbutyldiphenylsilyl-2,3,4,2′,3′,4′-hexa-O-benzyl-α,α-D-trehalose (56)

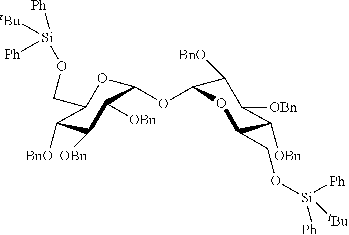

To a stirred suspension of D-trehalose (2 g, 5.85 mmol, 1 eq) and imidazole (0.39 g, 5.29 mmol, 0.9 eq) in dry DMF (10 mL) was added tert-butyl diphenylchlorosilane (TBDPS-Cl) (3 mL, 11.5 mmol, 2.0 eq) at RT. After stirring for 36 hours, TLC (2:1 ethyl acetate/methanol), indicated the complete consumption of starting materials and the formation of the product (R_{f} 0.35) as well as the mono TBDPS-trehalose (R_{f} 0.2). The reaction mixture was concentrated in vacuo and purification attained via silica gel chromatography (2:1, ethyl acetate/isopropanol) to give the desired product as a mixture of mono and di-TBDPS protected compounds and a white solid. (2.3 g, 55%), which were used without further purification. This mixture was dissolved in anhydrous DMF (25 mL), and sodium hydride (60% dispersed in mineral oil) (700 mg, 29.1 mmol) was added portionwise for a period of 10 min at 0° C. Benzyl bromide (2 mL, 11.6 mmol, 6 eq) was then added dropwise and the mixture left to stir under an atmosphere of argon at room temperature. After an 18 h period, TLC (5:1 petrol/ethyl acetate) indicated the formation of product (R_{f} 0.9) with complete consumption of the starting material (R_{f} 0). The reaction mixture was quenched by the slow addition of methanol (150 mL) and stirred for 30 min, at which point the resulting solution was concentrated in vacuo. The residue was dissolved in DCM (800 mL), washed with water and brine, filtered and concentrated in vacuo. Purification by column chromatography (petrol/ethyl acetate, 10:1) afforded 56 and 57 as a 2:1 mixture of products (by NMR), which were not fully separated (2.987 g, 41% over two steps) as a viscous clear oil. Further column chromatography yielded the titled compound (300 mg, 5% over two steps); [α]_{D}²⁵+23.6 (c=1.0 in CHCl₃); ¹H NMR (400 MHz, CHLOROFORM-d) δ ppm 1.28 (18H, s, 2×C(CH₃)), 3.75 (2H, dd, J_{2,3}=9.8 Hz, J_{1,2}=3.8 Hz, H-2, H-2′), 3.79 (2H, dd, J_{6a,6b}=10.5 Hz, J_{5,6b}=1.8 Hz, H-6b, H-6b′), 3.92 (2H, dd, J_{6a,6b}=10.6 Hz, J_{5,6a}=2.8 Hz, H-6a, H-6a′), 4.03 (2H, at, J_{3,4}=J_{4,5}=9.6 Hz, H-4, H-4′), 4.18-4.28 (4H, m, H-5, H-5′, H-3,H-3′), 4.72 (2H, d, J=11.8 Hz, 2×OCH₂Ph), 4.80 (2H, d, J=11.9 Hz, 2×OCH₂Ph), 4.89 (2H, d, J=10.9 Hz, 2×OCH₂Ph), 5.04 (2H, d, J=10.6 Hz, 2×OCH₂Ph), 5.10 (2H, d, J=10.6 Hz, 2×OCH₂Ph), 5.15 (2H, d, J=10.6 Hz, 2×OCH₂Ph), 5.39 (2H, d, J_{1,2}=3.5 Hz, H-1, H-1′), 7.13-7.63 (40H, m, Ar—H) 7.79-7.93 (10H, m, Ar—H); ¹³C NMR (101 MHz, CHLOROFORM-d) δ ppm 19.5 (6 C, s, 3×C(CH₃)₃), 27.0 (2C, s, 2×C(CH₃)₃), 65.6 (2C, C-6, C-6′), 71.7 (2C, C-5, C-5′), 72.9 (2C, OCH₂Ph), 75.3 (2C, OCH₂Ph), 76.0 (2C, OCH₂Ph), 77.7 (2C, C-4, C-4′), 80.3 (2C, C-2, C-2′), 82.1 (2C, C-3, C-3′), 94.2 (2C, C-1, C-1′), 126.1, 127.0, 127.2, 127.4, 127.7, 127.7, 127.8, 127.9, 128.0, 128.1, 128.3, 128.3, 128.6, 128.9, 129.2, 129.8, 129.9, 133.5, 133.6, 133.7, 135.6, 135.8, (6×OCH₂Ph, 4×OTBDPS), 137.9, 138.2, 138.4, 138.7, 138.9, (10 C, 6×1C, OCH₂Ph, 4×1C, OTBDPS); IR (thin film): ν=3069.3, 2930.1, 2856.3 (C═CH), 1453.7, 1427.6, (C═C), 1219.4, 111.2, 1069.3, 1027.4, 824.0, 772.5 cm⁻¹ m/z (ES⁺) 1376.6 (M+NH₄⁺). Isotopic distribution: Species observed (M+Na⁺), peaks observed 1381.59 (91.5%), 1382.59 (100%), 1383.59 (48.3%), 1384.59 (17.8%), 1385.60 (5.8%), 1386.59 (1.7%) peaks calculated 1381.62 (95.5%), 1382.62 (100%), 1383.62 (59.8%), 1384.62 (25.3%), 1385.63 (8.0%).

### 2,3,4,2′,3′,4′-Hexa-O-benzyl-α,α-D-trehalose (58)²¹

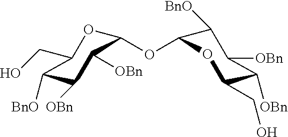

The 2:1 mixture of 56 and 57 (1.01 g, 0.83 mmol) was dissolved in DMF (10 mL) and to this was added TBAF dropwise (70% in H₂O) (1 mL, 2.87 mmol, 3.5 eq). Reaction was stirred for 3 h at 60° C. TLC (2:1 petrol/ethyl acetate) indicated products (R_{f} 0.4) and (R_{f} 0.05) and complete consumption of starting material (R_{f} 1.0). Crude product was washed with satd brine and NaHCO₃, extracted into dichloromethane and concentrated in vacuo. Purification by column chromatography (2:1 petrol/ethyl acetate followed by 1:1 petrol/ethyl acetate and ethyl acetate) yielded 58 (202.5 mg, 36%, based on 2:1 ratio of starting material) as a clear oil and 59 (156 mg, 58% based on 2:1 ratio of starting material) and recovered starting material (130 mg, 7%). Combined yield of both products was 61%.

[α]_{D}²⁵+88.1 (c=1.0 in CHCl₃) [Lit. [α]_{D}²⁵+104 (c=1.6 in CHCl₃)]²′; ¹H NMR (400 MHz, CHLOROFORM-d) δ ppm 3.55 (2H, dd, J_{2,3}=9.8 Hz, J_{1,2}=3.1 Hz, H-2, H-2′), 3.59-3.64 (6H, m, H-6a, H-6a′, H-6b, H-6b′), 3.61 (2H, at, J_{3,4}=J_{4,5}=10.1 Hz, H-4, H-4′), 4.05-4.13 (4H, m, H-3, H-3′, H-5, H-5′), 4.67 (2H, d, J=10.9 Hz, 2×OCH₂Ph), 4.68 (2H, d, J=11.9 Hz, 2×OCH₂Ph), 4.73 (2H, d, J=11.9 Hz, 2×OCH₂Ph), 4.90 (2H, d, J=10.9 Hz, 2×OCH₂Ph), 4.91 (2H, d, J=11.4 Hz, 2×OCH₂Ph), 5.02 (2H, d, J=10.8 Hz, 2×OCH₂Ph), 5.16 (2H, d, J₁₂=3.8 Hz, H-1, H-1), 7.15-7.52 (30H, m, Ar—H); ¹³C NMR (101 MHz, CHLOROFORM-d) δ ppm 61.4 (2C, C-6, C-6′), 71.4 (2C, C-5, C-5′), 72.9 (2C, 2×OCH₂Ph), 75.0 (2C, 2×OCH₂Ph), 75.6 (1C, 2×OCH₂Ph), 76.7 (2C, s, C-4, C-4′), 79.4 (2C, C-2, C-2′), 81.6 (2C, C-3, C-3′), 93.9 (2C, C-1, C-1′), 123.8, 127.5, 127.6, 127.7, 127.9, 128.1, 128.4, 128.5 (6×OCH₂Ph), 138.0, 138.2, 138.7 (6×1C, 6×OCH₂Ph); MS m/z (ESI⁺) 900.4 (M+NH₄⁺).

### 6,6′-Dihexanoyl-O-2,3,4,2′,3′,4′-hexa-O-benzyl-α,α-D-trehalose (61)

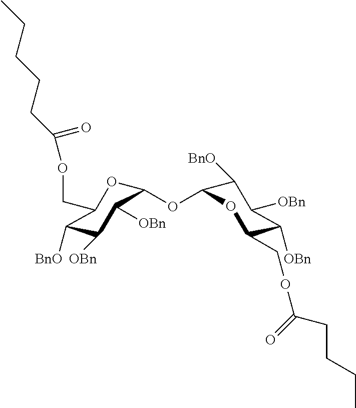

58 (1 g, 1.13 mmol, 1 eq) was dissolved in anhydrous pyridine (2 mL) and to this was added hexanoyl chloride (0.5 mL, 3.8 mmol, 3.3 eq). Reaction was stirred for 30 min at RT upon which time TLC (2:1 petrol/ethyl acetate) indicated complete conversion from starting material (R_{f} 0.05) to product (R_{f} 0.9). Reaction was washed with satd. NaHCO₃ solution and concentrated in vacuo. Column chromatography (10:1 petrol/ethyl acetate, 1% triethylamine) yielded the desired product as a clear oil (1.12 g, 91%). [α]_{D}²⁵+75.6 (c=0.39 in CHCl₃); ¹H NMR (400 MHz, CHLOROFORM-d) δ ppm 0.86 (6H, t, J=6.9 Hz, 2×OCO(CH₂)₄CH₃), 1.23-1.29 (8H, m, 2×OCO(CH₂)₂(CH₂)₂CH₃), 1.54-1.65 (8H, m, 2×OCO(CH₂)₂(CH₂)₂CH₃), 3.54 (4H, at, J_{3,4}=J_{4,5}=9.6 Hz, H-4, H-4′), 3.57 (4H, dd, J_{2,3}=9.6 Hz, J_{1,2}=3.5 Hz, H-2, H-2′), 4.06 (4H, at, J_{2,3}=J_{3,4}=9.3 Hz, H-3, H-3′), 4.06 (4H, dd, J_{6a,6b}=12.3 Hz, J_{5,6a}=2.0 Hz, H-6a, H-6a′), 4.16 (2H, dd, J_{6a,6b}=12.3 Hz, J_{5,6b}=3.3 Hz H-6b, H-6b′), 4.24 (2H, ddd, J_{4,5}=10.0 Hz, J_{5,6b}=3.4 Hz, J_{5,6a}=2.0 Hz, H-5, H-5′), 4.53 (2H, d, J=10.6 Hz, OCH₂Ph), 4.69 (2H, d, J=12.1 Hz, OCH₂Ph), 4.73 (2H, d, J=11.9 Hz, OCH₂Ph), 4.88 (4H, d, J=10.6 Hz, OCH₂Ph), 4.88 (3H, d, J=10.9 Hz, OCH₂Ph), 5.02 (2H, d, J=10.6 Hz, OCH₂Ph), 5.18 (2H, d, J_{1,2}=3.5 Hz, H-1, H-1′), 7.04-7.51 (40H, m, Ar—H); ¹³C NMR (101 MHz, CHLOROFORM-d) δ ppm 13.9 (2C, OCO(CH₂)₄CH₃), 22.3 (2C, OCO(CH₂)₄CH₂CH₃), 24.5 (2C, OCO(CH₂)₄CH₂CH₂CH₃), 31.3 (2C, OCOCH₂CH₂ (CH₂)₂CH₃), 34.0 (2C, OCOCH₂(CH₂)₃CH₃), 62.5 (2C, C-6, C-6′), 69.1 (2C, C-5, C-5′), 72.9 (2C, OCH₂Ph), 75.2 (4 C, 2×OCH₂Ph), 75.7 (2C, OCH₂Ph), 77.4 (2C, C-4, C-4′), 79.2 (2C, C-2, C-2′), 81.6 (2C, C-3, C-3′), 94.0 (2C, C-1, C-1′), 127.5, 127.7, 127.8, 127.9, 128.1, 128.4, 128.4 (6×OCH₂Ph); 137.8, 137.8, 138.5 (6×1C=6×OCH₂Ph); 173.5 (2C, 2×C═O); IR (thin film): ν=3419 br (OH), 2956, 2925, 2854 (C═CH), 1733 (C═O), 1637, 1456 (C═C), 1219, 1177, 1150, 1101, 1078, 1053, 1026, 990, 772 cm⁻¹; MS m/z (ESI⁺) 1096.5 (M+NH₄); Isotopic distribution: Species observed (M+Na⁺), peaks observed 1101.53 (100%), 1102.53 (72.8%), 1103.54 (28.8%), 1104.54 (8.1%), 1105.54 (1.8%), peaks calculated 1101.52 (100%), 1102.52 (74.4%), 1103.53 (30.0%), 1104.53 (8.3%), 1105.53 (1.9%), 1105.49 (1.4%).

### 6,6′-O-dihexanoyl-α,α-D-trehalose (60)

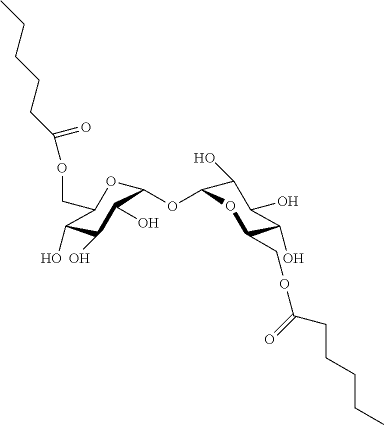

61 (474 mg, 0.44 mmol) was dissolved in 20 mL ethanol and the solvent was degassed under alternating reduced pressure and argon. 10% Pd/C (300 mg) was added to the solution, which was then activated through repeated vacuum, flush cycles with 2 hydrogen balloons. Reaction was stirred at RT for 16 h, upon which time ESI mass spec showed complete conversion to the desired product. TLC (2:1 ethyl acetate/methanol) indicated complete conversion from starting material (R_{f} 1) to product (R_{f} 0.6). Reaction was concentrated in vacuo to produce the desired product as a crystalline white solid (120.0 mg, 55%). M.p.=157.7-159.0° C.; [α]_{D}²⁵+152.3 (c=0.13 in H₂O); ¹H NMR (500 MHz, DEUTERIUM OXIDE) δ ppm 0.78 (6H, t, J=6.9 Hz, 2×OCO(CH₂)₄CH₃), 1.11-1.29 (8H, m, 2×OCO(CH₂)₂(CH₂)₂CH₃) 1.53 (4H, quin, J=7.3 Hz, 2×OCOCH₂(CH₂)(CH₂)₂CH₃), 2.33 (4H, t, J=7.3 Hz, 2×OCOCH₂(CH₂)₄CH₃), 3.39 (2H, at, J_{3,4}=J_{4,5}=9.6 Hz H-4, H-4′), 3.54 (2H, dd, J_{2,3}=9.8 Hz, J_{1,2}=3.8 Hz, H-2, H-2′), 3.76 (2H, at, J_{2,3}=J_{3,4}=9.5 Hz, H-3, H-3′), 3.92 (2H, ddd, J_{4,5}=10.1 Hz, J_{5,6a}=5.0 Hz, J_{5,6b}=2.2 Hz, H-5, H-5′), 4.21 (2H, dd, J_{6a,6b}=12.3, J_{5,6a}=5.4 Hz, H-60, 4.33 (2H, dd, J_{6a,6b}=12.3 Hz, J_{5,6b} 2.2 Hz, H-6b), 5.05 (2H, d, J_{1,2}=3.8 Hz, H-1, H-1′); ¹³C NMR (126 MHz, DEUTERIUM OXIDE) δ ppm 13.1 (2C, 2×OCO(CH₂)₄CH₃), 21.6 (2C, 2×OCO(CH₂)₃CH₂CH₃), 24.0 (2 C, 2×OCO(CH₂)₂CH₂CH₂CH₃), 30.5 (2C, 2×OCO CH₂CH₂(CH₂)₂CH₃), 33.8 (2C, 2×OCOCH₂(CH₂)₄CH₃), 62.9 (2C, C-6, C-6′), 69.7 (2C, C-5, C-5′), 70.0 (2C, C-4, C-4′), 70.92 (2C, C-2, C-2′), 72.4 (2C, C-3, C-3′), 93.3 (2C, C-1, C-1′), 176.8 (2C, 2×C═O); MS m/z (ESI) 561.3 (M+Na⁺); HRMS (ESI) calcd. for C₂₄H₄₂O₁₃ (M+Na⁺): 561.2523. Found: 561.2513.

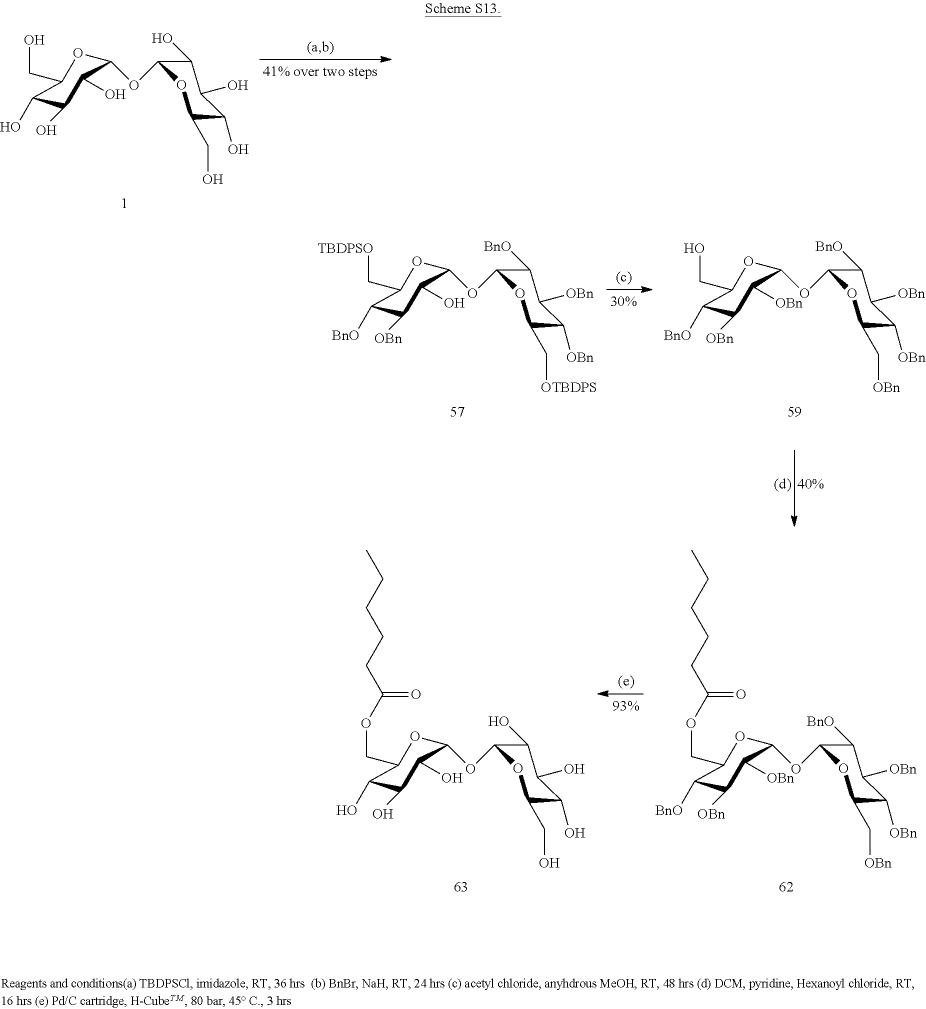

### 6-O-tertbutyldiphenylsilyl-2,3,4,2′,3′,4,′6′-hepta-O-benzyl-α,α-D-trehalose (57)

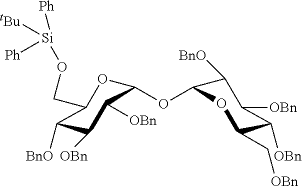

D-trehalose (7.4 g, 21.75 mmol, 1 eq) was dissolved in anhydrous DMF (40 mL). To this was added tert-butyl diphenylchlorosilane (TBDPS-C1) (5 mL, 18 mmol, 0.9 eq) and imidazole (1.4 g, 21 mmol, 0.95 eq). Solution was stirred at RT under Ar atmosphere for 18 h. TLC (2:1 petrol/ethyl acetate) indicated primarily starting material (R_{f} 0.1) and a small amount of the mono-TBDPS-trehalose. Additional TBDPS-Cl was added (2.5 mL, 9 mmol, 0.45 eq) and reaction was left for a further 18 hours. NaH (8 g, 339 mmol, 15 eq) and benzyl bromide (25 mL, 145 mmol, 7 eq) were added in situ. Reaction was stirred for a further 24 hours under argon, until the desired product could be detected by TLC (5:1 petrol/ethyl acetate) (R_{f} 0.85). Column chromatography yielded the desired product as a slightly yellow oil. (4.83 g, 19% over two steps).

[α]_{D}²⁵+16.13 (c=0.88 in CHCl₃); ¹H NMR (400 MHz, CHLOROFORM-d) δ ppm 1.14 (9H, s, 3×C(CH₃)₃), 3.47 (1H, dd, J_{6a,6b}=10.2 Hz, J_{5,6b}=1.8 Hz, H-6b′), 3.60 (1H, dd, J J_{6a,6b}=10.2 Hz, J_{5,6a}=2.7 Hz, H-6a′), 3.64 (1H, dd, J_{2,3}=7.7 Hz, J_{1,2}=4.2 Hz, H-2′), 3.67 (1H, dd, J_{2,3}=7.8 Hz, J_{1,2}=3.8 Hz, H-2), 3.72 (1H, dd, J_{6a,6b}=9.7 Hz, J_{5,6a}=3.7 Hz, H-6a), 3.78 (1H, at, J_{3,4}=J_{4,5}=9.4 Hz, H-4′), 3.87 (1 H, dd, J_{6a,6b}=10.4 Hz, J_{5,6b}=2.3 Hz, H-6b), 3.97 (1H, at, J_{3,4}=J_{4,5}=9.5 Hz, H-4), 4.13-4.17 (3H, m, H-5, H-3, H-3′) 4.27 (1H, ddd, J_{4,5}=10.0 Hz, J_{5,6a}=2.8, J_{5,6b}=2.7 Hz, H-5′), 4.47 (1H, d, J=11.9 Hz, 1×OCH₂Ph), 4.52 (1H, d, J=10.6 Hz, 1×OCH₂Ph), 4.57 (1H, d, J=12.6 Hz, 1×OCH₂Ph), 4.58 (1H, d, J=12.1 Hz, 1×OCH₂Ph), 4.87 (1H, d, J=12.1 Hz, 1×OCH₂Ph), 4.90 (1H, d, J=11.1 Hz, 1×OCH₂Ph), 4.94 (1H, d, J=9.3 Hz, 1×OCH₂Ph), 4.96 (1H, d, J=11.6 Hz, 1×OCH₂Ph), 4.99 (1H, d, J=9.6 Hz, 1×OCH₂Ph), 5.01 (1H, d, J=9.6 Hz, 1×OCH₂Ph), 5.04 (1H, d, J=12.2 Hz, 1×OCH₂Ph), 5.07 (1H, d, J=11.0 Hz, 1×OCH₂Ph), 5.09 (1H, d, J=10.6 Hz, 1×OCH₂Ph), 5.28 (1H, d, J_{1,2}=3.8 Hz, H-1′), 5.37 (1H, d, J_{1,2}=3.8 Hz, H-1), 7.00-7.61 (45H, m, Ar—H); ¹³C NMR (101 MHz, CHLOROFORM-d) δ ppm 20.7 (3×C(CH₃)₃), 28.1 (1×C(CH₃)₃), 62.2 (C-6), 68.2 (C-6′), 70.6 (C-5′), 71.69 (C-5), 72.8 (OCH₂Ph), 72.9 (OCH₂Ph), 73.6 (OCH₂Ph), 75.1 (OCH₂Ph), 75.3 (OCH₂Ph), 75.7 (OCH₂Ph), 75.9 (OCH₂Ph), 77.70 (C-4 or C-4′), 77.7 (C-4 or C-4′), 79.5 (C-2′), 80.1 (C-2), 81.9 (C-3 or C-3′), 81.9 (C-3 or C-3′), 94.2 (2C, C-1, C-1′), 126.0, 126.3, 126.4, 127.1 127.2, 127.3, 127.4, 127.5, 127.6, 127.7, 127.8, 127.9, 128.0, 128.0, 128.1, 128.2, 128.4, 128.4, 128.5, 128.6, 128.9, 129.1, 129.5, 129.7 (7×OCH₂Ph), 138.0, 138.4, 138.5, 138.6, 138.9, 139.0, 139.6 (7×1C, 7×OCH₂Ph); IR (thin film): ν=2925, 2875 (C═CH), 1454, 1444 (C═C), 698 cm⁻¹; MS m/z (ESI⁺) 1228.6 (M+NH₄⁺); Isotopic distribution: Species observed (M+Na⁺), peaks observed 1233.53 (100%), 1234.53 (86.6%), 1235.53 (43.1%), 1236.53 (14.5%), 1237.53 (4.0%), 1238.54 (1.1%) peaks calculated 1233.55 (100.0%), 1234.55 (89.7%), 1235.55 (45.2%), 1236.56 (16.0%), 1237.56 (4.2%), 1238.56 (0.6%)

### 2,3,4,6,2′,3′,4′-hepta-O-benzyl-α,α-D-trehalose (59)²¹

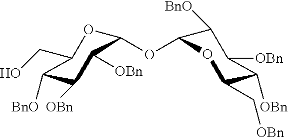

57 (4 g, 3.3 mmol, 1 eq) was dried under reduced pressure for 1 hour and dissolved in anhydrous methanol (20 mL). To this was added acetyl chloride (5 ml), generating acetic acid in situ. Reaction was stirred in acid for 48 hours, upon which time TLC (2:1 petrol/ethyl acetate) indicated product (R_{f} 0.4) and complete consumption of starting material (R_{f} 0.95). Reaction was quenched with water and poured into a satd. NaHCO₃ soln. When gas ceased to evolve, reaction was extracted into DCM and concentrated in vacuo. Column chromatography (10:1 to 1:1 petrol/ethyl acetate) yielded the desired product (1.1 g, 30%, 9% over three steps). [α]_{D}²⁵+54.3 (c=0.77 in CHCl₃); ¹H NMR (500 MHz, CHLOROFORM-d) δ ppm 3.38 (1H, dd, J_{6a,6b}=10.7, J_{5,6b}=2.2 Hz, H-6b), 3.51 (1H, dd, J_{6a,6b}=10.4 Hz, J_{5,6a}=3.2 Hz, H-6a), 3.52 (1H, dd, J_{6a,6b}=9.6 Hz, J_{5,6a}=3.6 Hz, H-6a′) 3.57-3.61 (4H, m, H-2′, H-2, H-4, H-6b′), 3.68 (1H, at, J_{3,4}=J_{4,5}=9.6 Hz, H-4′), 4.03 (1H, at, J_{2,3}=J_{3,4}=9.5 Hz, H-3), 4.06 (1H, at, J_{2,3}=J_{3,4}=9.3 Hz, H-3′), 4.05-4.09 (1H, m, H-5′), 4.15 (1H, ddd, J_{4,5}=10.0 Hz, J_{5,6a}=3.5 Hz, J_{5,61})=2.4 Hz, H-5), 4.38 (1H, d, J=12.0 Hz, 1×OCH₂Ph), 4.46 (1H, d, J=11.0 Hz, 1×OCH₂Ph), 4.54 (1H, d, J=12.0 Hz, 1×OCH₂Ph), 4.65 (1H, d, J=11.0 Hz, 1×OCH₂Ph), 4.69 (2H, d, J=10.0 Hz, 2×OCH₂Ph), 4.71 (2H, d, J=2×OCH₂Ph), 4.82 (1H, d, J=10.7 Hz, 1×OCH₂Ph), 4.87 (1H, d, J=11.0 Hz, 1×OCH₂Ph), 4.88 (2H, d, J=10.7 Hz, 2×OCH₂Ph), 4.99 (1H, d, J=11.0 Hz, 1×OCH₂Ph), 5.00 (1H, d, J=10.7 Hz, 1×OCH₂Ph), 5.18 (1H, d, J_{1,2}=3.5 Hz, H-1), 5.19 (1H, d, J_{1,2}=3.8 Hz, H-1′), 7.25-2.40 (35H, m, Ar—H); ¹³C NMR (126 MHz, CHLOROFORM-d) δ 61.5 (C-6), 68.12 (C-6′), 70.6 (C-5), 71.1 (C-5′), 72.1 (OCH₂Ph), 72.8 (OCH₂Ph), 72.8 (OCH₂Ph), 73.5 (OCH₂Ph), 75.0 (OCH₂Ph), 75.5 (2C, 2×OCH₂Ph), 77.0 (C-4′), 77.6 (C-4), 79.3 (C-2 or C-2′), 79.5 (C-2 or C-2′), 81.5 (C-3), 81.7 (C-3′), 94.1 (C-1 or C-1′), 94.3 (C-1 or C-1′), 127.4, 127.5, 127.6, 127.6, 127.7, 127.8, 127.9, 128.1, 128.3, 128.3, 128.4, 128.4 (7×OCH₂Ph), 134.6, 137.7, 138.1, 138.1, 138.3, 138.7, 138.8 (7×1C, 7×OCH₂Ph). MS m/z (ES⁺) 990.4 (M+NH₄⁺)

### 6-O-Hexanoyl-2,3,4,2′,3′,4,6′-hepta-O-benzyl-α,α-D-trehalose (62)

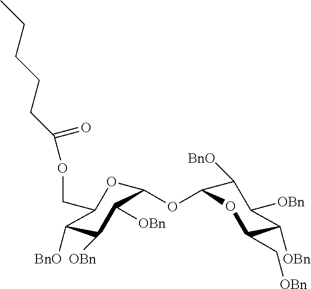

59 (116 mg, 0.119 mmol, 1 eq) was dissolved in anhydrous DCM (10 mL) with anhydrous pyridine (2 mL) and to this was added hexanoyl chloride (0.02 mL, 0.15 mmol, 1.25 eq), which, upon addition caused the reaction mixture to yellow. Reaction was stirred for 16 h at RT upon which time TLC (2:1 petrol/ethyl acetate) indicated incomplete conversion from starting material (R_{f} 0.1) to product (R_{f} 0.6). An additional portion of hexanoyl chloride (0.05 mL, 0.375 mmol, 3.125 eq) was added and reaction was left at RT for an additional 24 h. Reaction was washed with satd NaHCO₃ solution and concentrated in vacuo. Column chromatography yielded the desired product as a clear oil (52.1 mg, 40%).

[α]_{D}²⁵+30.4 (c=0.56 in CHCl₃); ¹H NMR (500 MHz, CHLOROFORM-d) δ ppm 0.86 (3H, t, J=6.8 Hz, OCO(CH₂)₄CH₃), 1.20-1.30 (2H, m OCO(CH₂)₃CH₂CH₃), 2.16-2.31 (4H, m, OCOCH₂(CH₂)₂CH₂CH₃), 2.24 (2H, t, J=7.3 Hz, OCO OCOCH₂(CH₂)₃CH₃), 3.38 (1H, dd, J_{6a,6b}=10.7 Hz, J_{5,6b}=1.6 Hz, H-6b), 3.52 (1H, dd, J_{6a,6b}=10.8 Hz, J_{5,6a}=3.7 Hz, H-6a), 3.53 (1H, at, J_{3,4}=J_{4,5}=9.2 Hz, H-4), 3.56 (1H, dd, J_{2,3}=9.4 Hz, J_{1,2}=3.5 Hz, H-2), 3.60 (1H, dd, J_{2,3}=9.8 Hz, J_{1,2}=3.8 Hz, H-2′), 3.68 (1H, at, J_{3,4}=J_{4,5}=9.6 Hz, H-4′), 4.03 (1H, at, J_{2,3}=J_{3,4}=9.2 Hz, H-3), 4.06 (1H, at, J_{2,3}=J_{3,4}=8.8 Hz, H-3′), 4.06 (1H, dd, J_{6a,6b}=10.8 Hz, J_{5,6b}=2.2 Hz, H-6b′), 4.11-4.18 (2H, m, H-6a′, H-5′), 4.25 (1H, ddd, J_{4,5}=9.9 Hz, J_{5,6b}=3.4 Hz, J_{5,6a}=2.2 Hz, H-5), 4.38 (1H, d, J=12.3 Hz, 1×OCH₂Ph), 4.46 (1H, d, J=10.7 Hz, 1×OCH₂Ph), 4.52 (1H, d, J=11.0 Hz, 1×OCH₂Ph), 4.54 (1 H, d, J=12.0 Hz, 1×OCH₂Ph), 4.68 (2H, d, J=11.7 Hz, 2×OCH₂Ph), 4.72 (2H, d, J=12.0 Hz, 2×OCH₂Ph), 4.81 (1H, d, J=10.7 Hz 1×OCH₂Ph), 4.86 (3H, d, J=11.1 Hz, 3×OCH₂Ph), 4.99 (1H, d, J=10.8 Hz, 1×OCH₂Ph), 5.01 (1H, d, J=10.7 Hz, 1×OCH₂Ph), 5.20 (1H, d, J₁₂=3.3 Hz, H-1), 5.21 (1H, d, J_{1,2}=3.2 Hz, H-1′), 7.21-7.35 (35H, m, Ar—H); ¹³C NMR (126 MHz, CHLOROFORM-d) δ ppm 13.9 (OCO(CH₂)₄CH₃), 22.2 (1C, 1×OCO(CH₂)₃CH₂CH₃), 24.5 (1C, 1×OCO(CH₂)₂CH₂CH₂CH₃), 31.2 (1C, OCOCH₂CH₂(CH₂)₂CH₃), 34.0 (1C, OCOCH₂(CH₂)₃CH₃), 62.5 (C-6′), 68.1 (C-6), 69.0 (C-5), 70.7 (C-5′), 72.7 (OCH₂Ph), 72.9 (OCH₂Ph), 73.5 (OCH₂Ph), 75.1 (OCH₂Ph), 75.1 (OCH₂Ph), 75.6 (OCH₂Ph), 75.7 (OCH₂Ph), 77.5 (C-4 or C-4′), 77.6 (C-4 or C-4′), 79.2 (C-2 or C-2′), 79.4 (C-2 or C-2′), 81.6 (C-3 or C-3′), 81.8 (C-3 or C-3′), 94.0 (C-1 or C-1′), 94.4 (C-1 or C-1′), 127.3, 127.5, 127.5, 127.6, 127.6, 127.7, 127.8, 127.9, 127.9, 127.9, 128.1, 128.3, 128.4, 128.4, 128.5 (7×OCH₂Ph), 137.7, 137.9 (2C), 138.0, 138.3, 138.6, 138.8, (7×1C=7×OCH₂Ph) 173.5 (C═O); IR (thin film): ν=3063, 3031, 2925, 2855 (C═CH), 1735 (C═O), 1586, 1496, 1454, 1360 (C═C), 1262, 1219, 1156, 1100, 772 cm⁻¹; MS m/z (ESI⁺) 1093.4 (M+Na⁺); Isotopic distribution: species observed (M+Na), peaks observed 1093.47 (100%), 1094.48 (73.7%) 1095.49 (27.4%), 1096.50 (6.4%) peaks calculated 1093.50 (100%), 1094.52 (73.8%), 1096.51 (8.1%)

### 6-O-Hexanoyl-α,α-D-trehalose (63)

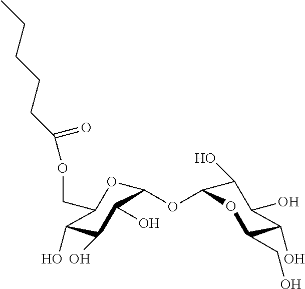

62 (6.2 mg, 0.019 mmol) was dissolved in 20 mL ethanol and circulated through a Pd/C cartridge in the H-Cube™ at 80 bar, 45° C. and 1 mL/min. Reaction was monitored by mass spec and after 3 hours TLC (2:1 ethyl acetate/methanol) indicated complete conversion from starting material (R_{f} 1) to product (R_{f} 0.4). Reaction was concentrated in vacuo and purified via column chromatography (3:1 ethyl acetate:methanol) to produce 63 as a white crystalline solid (2.6 mg, 93%). M.p.=135-137° C.;

[α]_{D}²⁵=111.3 (c=1.0, MeOH). ¹H NMR (500 MHz, DEUTERIUM OXIDE) δ ppm 0.79 (3H, t, J=6.9 Hz, OCO(CH₂)₄CH₃), 1.17-1.26 (4H, m, OCOCH₂CH₂(CH₂)₂CH₃), 1.54 (2H, quin, J=7.3 Hz, OCOCH₂CH₂(CH₂)₂CH₃), 2.35 (2H, t, J=7.4 Hz, OCOCH₂CH₂(CH₂)₂CH₃), 3.36 (1H, at, J_{3,4}=J_{4,5}=9.5 Hz, H-4), 3.41 (1H, at, J_{3,4}=J_{4,5}=9.6 Hz, H-4′), 3.55 (1H, dd, J_{2,3}=9.9 Hz, J_{1,2}=3.8 Hz, H-2), 3.57 (1H, dd, J_{2,3}=10.1 Hz, J_{1,2}=3.8 Hz, H-2′), 3.66-3.82 (5H, m, H-6a, H-6b, H-5, H-3, H-3′), 3.94 (2H, ddd, J_{4,5}=10.2 Hz, J_{5,6a}=4.8 Hz, J_{5,6b}=2.0 Hz, H-5′), 4.22 (2H, dd, J_{6a,6b}=12.3 Hz, J_{5,6a}=5.0 Hz, H-6a′), 4.35 (2H, dd, J_{6a,6b}=12.3 Hz, J_{5,6b}=2.2 Hz, H-6b′), 5.07 (1H, d, J_{1,2}=3.9 Hz, H-1), 5.10 (1H, d, J_{1,2}=3.8 Hz, H-1′); ¹³C NMR (126 MHz, DEUTERIUM OXIDE) δ ppm 13.1 (1C, OCO(CH₂)₄CH₃), 21.6 (1C, OCO(CH₂)₃CH₂CH₃), 24.0 (1C, OCO(CH₂)₃CH₂CH₂CH₃), 30.5 (1C, OCOCH₂CH₂(CH₂)₂CH₃), 33.7 (1C, OCOCH₂(CH₂)₃CH₃), 60.5 (C-6), 62.9 (C-6′), 69.6 (C-5′), 69.6 (C-4 or C-4′), 69.9 (1 C-4 or C-4′), 70.9 (C-2 or C-2′), 70.9 (C-2 or C-2′), 72.2 (C-5), 72.3 (C-3 or C-3′), 72.6 (C-3 or C-3′), 93.2 (C-1′), 93.4 (C-1), 176.8 (C═O); MS m/z (ESI⁻) 475.2 (M+Cl); HRMS (ESI⁺) calcd. for C₁₈H₃₂O₁₂ (M+Na⁺): 463.1791. Found: 463.1793.

### Trehalose Quantum Dots

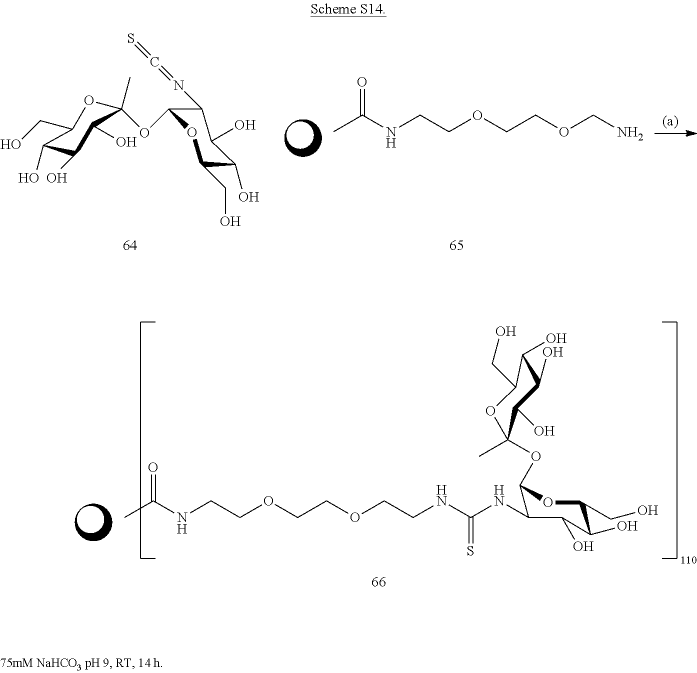

### 1-Deoxy-α-D-gluco-hept-2-ulopyranosyl 2-N-isothiocyanate-2-deoxy-α-D-glucopyranoside (64)

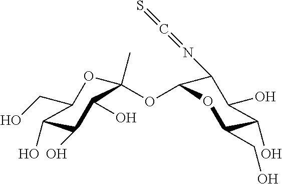

7 (2.5 mg, 0.007 mmol, 1 eq) was dissolved in 75 mM NaHCO₃ buffer pH 9 (200 μl). To this was added thiophosgene as a solution (20 μl of thiophosgene into 1 mL of chloroform). 100 μl of thiophosgene solution was added to reaction (3 mg, 0.26, 4.2 eq). The resulting biphasic mixture was stirred at room temperature for 3 hours upon which TLC (5 ethanol:3 NH₄OH:1 water) showed complete consumption of starting material (R_{f} 0.2) and conversion to a single product (R_{f} 0.65). Excess thiophosgene and chloroform were removed in vacuo and crude product was used without further purification.

### Trehalose Quantum Dots (66)

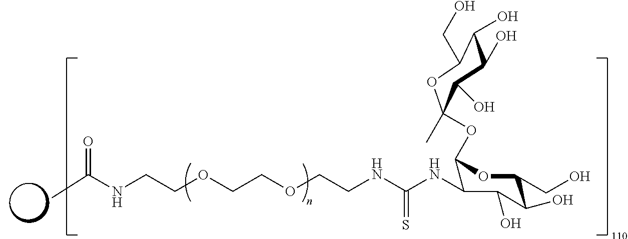

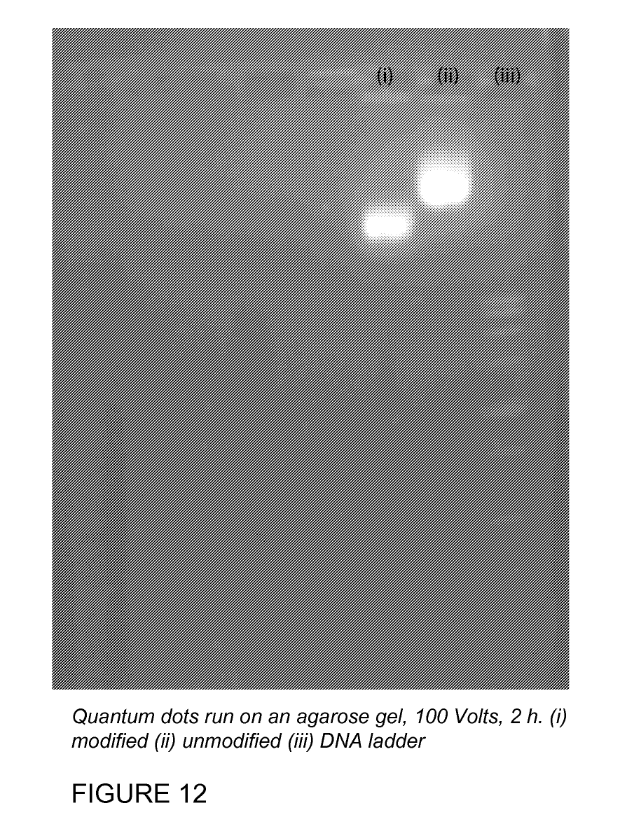

An 8 μM solution of CdSe—ZnS (50 μl) core-shell quantum dots (emission λₘₐₓ 655 nm) in borate buffer at pH 8 (Invitrogen) was buffer exchanged into water by repeated (×5) centrifugal filtration through a 10 kDa cutoff spin filter. Dots were then resuspended in water. 50 μl of this 8 μM quantum dot solution was then added to the solution of 64, and the total volume made up to 1.0 mL with 75 mM NaHCO₃ buffer at pH 9.0 (pH electrode). Reaction mixture was shaken at 4° C. for 14 hours. Excess sugar and salt was removed from the reaction mixture by size exclusion chromatography (PD 10 column, Amersham) with water as the mobile phase. The quantum dot solution was concentrated to 1 mL, using 10 Kda spin filter, and the concentration determined using previously reported procedures²² to be 0.44 μM (ε₃₅₀=3880000 M⁻¹ cm⁻¹). The modification of the quantum dots was confirmed using an agarose gel, as shown in FIG. 12, which shows a standardised curve for determining trehalose concentration on modified quantum dots using the phenol-sulphuric acid method. Shown is the absorbance at 490 nm after reaction with phenol and sulphuric acid against standardized concentrations of trehalose.

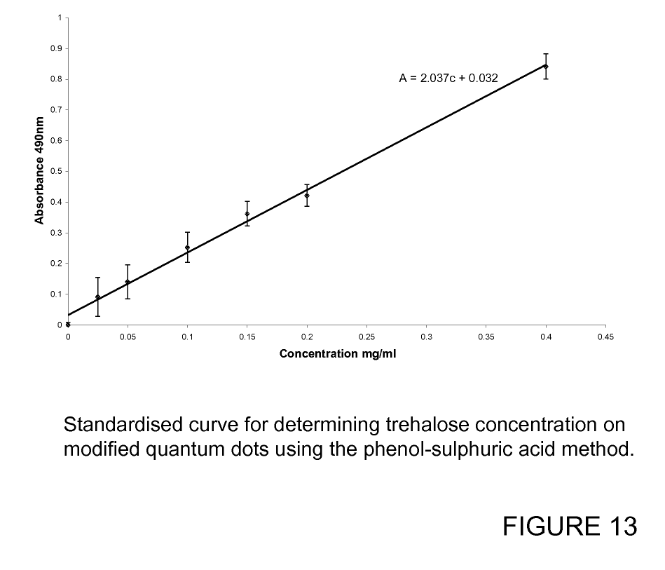

The carbohydrate loading on the quantum dots was determined using the phenol sulphuric acid method. An aliquot of the quantum dot solution (50 μl) was treated with concentrated sulphuric acid (75 μl) and aqueous phenol (5% w/w, 10 μl) and heated to 90° C. After 5 minutes the sample was cooled to room temperature and A₄₉₀ measured, referenced to a solution of carbohydrate modified quantum dots and acid. The concentration of trehalose was determined by comparison to a standardised curve (FIG. 13). The carbohydrate content per dot was calculated from the ratio of trehalose concentration to the concentration of ZnS—CdSe quantum dots and was found to be ˜110 sugars/dot.

### Synthesis of Trehalose-6-Phosphates

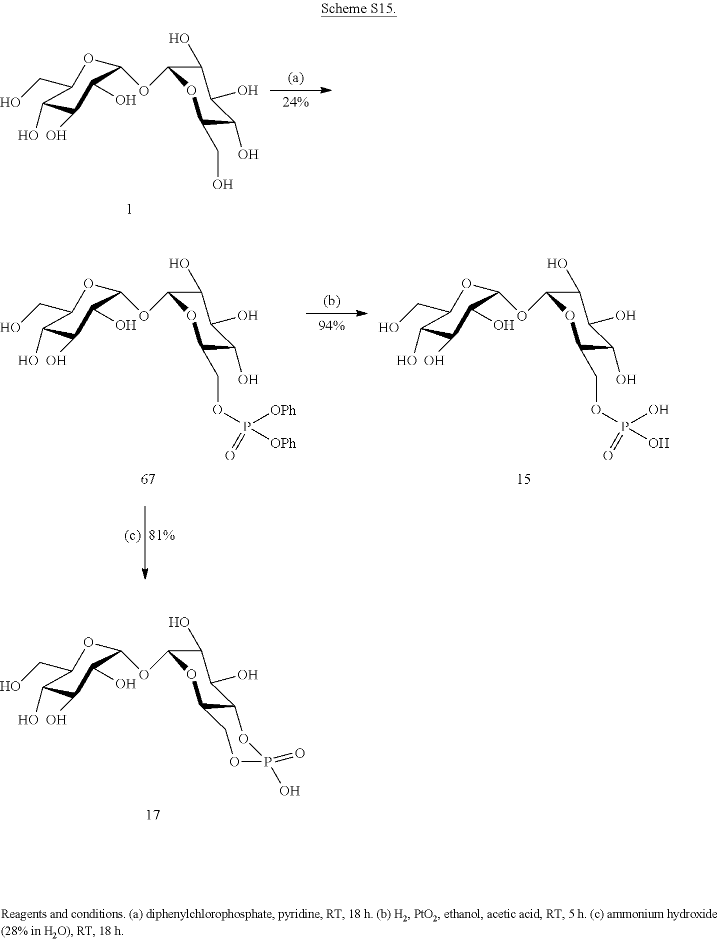

### 6-O-(diphenoxyphosphoryl)-D-trehalose (67)

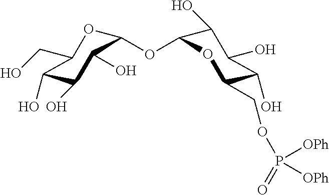

To a suspension of D-trehalose (7.50 g, 21.9 mmol, 1 eq) in anhydrous pyridine (100 mL) was added dropwise diphenylchlorophosphate (4.54 mL, 21.9 mmol, 1 eq). TLC (1:4:4, water/isopropanol/ethyl acetate) after 18 hours showed the presence of two products (desired product R_{f} 0.7). The reaction was quenched with methanol (10 mL). The reaction mixture was concentrated in vacuo, and the residue co-evaporated with toluene to remove pyridine. Silica gel chromatography (1:3:13 water/isopropanol/ethyl acetate) al lowed separation of the two products. Lyophilization yielded the desired compound (3.02 g, 24%) as a white amorphous solid.

[α]_{D}²²+63.9 (c=1.0 in MeOH); ¹H NMR (500 MHz, METHANOL-d) δ ppm 3.34 (1H, at, J_{3,4}=9.1 Hz, J_{4,5}=9.1 Hz, H-4′), 3.38 (1H, at, J_{3,4}=J_{4,5}=9.1 Hz, H-4), 3.43 (1H, dd, J_{2,3}=9.8 Hz, J_{1,2}=3.5 Hz, H-2′), 3.48 (1H, dd, J_{2,3}=9.8 Hz, J_{1,2}=3.8 Hz, H-2), 3.70 (1H, dd, J_{6a,6b} 12.0 Hz, J_{5,6a}=5.4 Hz, H-6a′), 3.80 (1H, at, J_{2,3}=J_{3,4}=9.5 Hz, H-3′), 3.81 (1H, t, J_{2,3}=J₃₋₄=9.1 Hz, H-3), 3.80-3.86 (2H, m, H-5′), 4.09 (1H, dt, J_{4,5}=10.1 Hz, J_{5,6a}=2.1 Hz, J_{5,6b}=2.1 Hz, H-5), 4.48 (1H, ddd, J_{6a,6b}=11.5 Hz, J_{P,6a}=7.1 Hz, J_{5,6a}=3.5 Hz, H-6a), 4.55 (1H, ddd, J_{6a,6b}=11.5 Hz, J_{P,6b} 6.8 Hz, J_{5,6b}=1.9 Hz, H-6b), 5.09 (1H, d, J_{1′,2′}=3.8 Hz, H-1′), 5.10 (1H, d, J_{1,2}=3.8 Hz, H-1), 7.21-7.31 (3H, m, Ar—Hₒᵣₜₕₒ, Ar—Hₚₐᵣₐ), 7.40-7.43 (2H, m, Ar—Hₘₑₜₐ); ¹³C NMR (126 MHz, METHANOL-d) δ ppm 62.6 (C-6′), 69.8 (1C, d, J_{P,6}=6.7 Hz, C-6), 71.2 (C-4′), 71.9 (C-4), 72.0 (1C, d, J_{P,5}=6.7 Hz, C-5), 73.0 (C-5′), 73.2 (C-2′), 73.9 (C-2), 74.4 (C-3′), 74.6 (C-3), 95.2 (C-1′), 95.3 (C-1), 121.4 (2C, d, J_{P,C}=4.8 Hz, Ar—Cₒᵣₜₕₒ), 126.8 (Ar—Cₚₐᵣₐ), 131.1 (Ar—Cₘₑₜₐ), 151.9 (1C, d, J_{P,C}=7.6 Hz, Ar—Cᵢₚₛₒ) 151.9 (1C, d, J_{P,C}=7.6 Hz, Ar—Cᵢₚₛₒ); ³¹P(¹H) NMR (162 MHz, METHANOL-d) δ ppm −11.9; IR (KBr disc): ν=1287 (P═O), 3271 br (OH) cm⁻¹; (ESI⁺) m/z 596.74 (M+Na); HRMS (ESI) m/z calcd. for C₂₄H₃₁O₁₄P (M+Na): 596.1344. Found: 596.1357.

### Trehalose-6-phosphate (15)²³

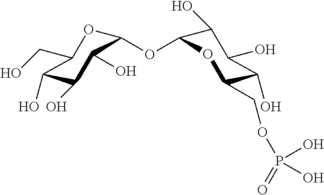

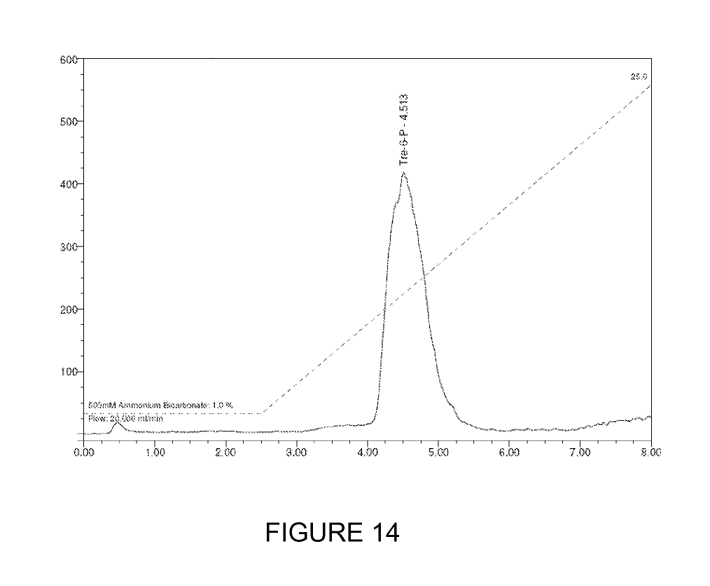

A suspension of 67 (50 mg, 0.072 mmol, 1 eq) and PtO₂ (5 mg, 0.02 mmol, 0.3 eq) in 75% aqueous ethanol (5 mL) with 0.5% glacial acetic acid (25 μL) was repeatedly degassed under high vacuum and the reaction vessel flushed with hydrogen. The reaction was maintained at RT with aggressive stirring under an atmospheric pressure of hydrogen for 5 hours after which TLC (1:4:4, water/isopropanol/ethyl acetate) showed the complete consumption of the starting material (R_{f} 0.7) and the formation of a single product (R_{f} 0). The reaction mixture was filtered through Celite® and the solvent was removed in vacuo. The crude solid was taken up in water (30 mL) and washed with ethyl acetate (2×20 mL). The aqueous layer was lyophilized and the compound purified using HPLC on an Applied Biosystems, Poros® HQ strongly basic anion exchange column (10 mm×100 mm, 50 μm). A gradient from 0 mM to 500 mM aqueous NH₄HCO₃ was used as the mobile phase at a flow rate of 20 mL/min and eluants were detected with an Evaporative Light Scattering (ELSD) detector, as shown in FIG. 14. Fractions containing the product were pooled and repeated lyophilization to removed residual NH₄HCO₃ afforded the title compound as a white amorphous solid (28.6 mg, 94%).

[α]_{D}¹⁸+150.3 (c=1.0 in H₂O), [lit. [α]_{D}²¹+151.2 (c=0.8 in H₂O)]²³; ¹H NMR (500 MHz, DEUTERIUM OXIDE) δ ppm 3.36 (1H, at, J_{3,4}=J_{4,5}=9.5 Hz, H-4′), 3.50 (1H, at, J_{3,4}=J_{4,5}=9.6 Hz, H-4), 3.56 (1H, dd, J_{2,3}=10.1 Hz, J_{1,2}=3.8 Hz, H-2), 3.59 (1H, dd, J_{2,3}=9.8 Hz, J_{1,2}=3.8 Hz, H-2′), 3.67 (1H, dd, J_{6a,6b}=11.8 Hz, J_{5,6b}=5.4 Hz, H-6a′), 3.71-3.83 (5H, m, H-3, H-3′, H-5′, H-6b′, H-5), 3.86 (1H, ddd, J_{6a,6b}=12.1 Hz, J_{P,6b}=5.4 Hz, J_{5,6b}=1.7 Hz, H-6b), 3.94 (1H, ddd, J_{6a,6b}=11.9 Hz, J_{P,6a}=7.7 Hz, J_{5,6a}=4.1 Hz, H-6a), 5.09 (1H, d, J_{1,2}=4.1 Hz, H-1′), 5.12 (1H, d, J_{1,2}=3.8 Hz, H-1); ¹³C NMR (126 MHz, DEUTERIUM OXIDE) δ ppm 60.5 (C-6′), 63.0 (d, J_{P,6}=4.8 Hz, C-6), 69.1 (C-4′), 69.7 (C-4), 70.9 (C-2), 71.1 (C-2′), 71.6 (1C, d, J_{P,5}=6.7 Hz, C-5), 72.1 (C-5′), 72.2 (C-3), 72.4 (C-3′), 93.3 (C-1), 93.4 (C-1′); ³¹P(¹H) NMR (202 MHz, DEUTERIUM OXIDE) δ ppm 3.63; MS (ESI⁻) m/z 421.5 (M−H⁺).

### 4,6-(monohydrogen)phosphoryl-D-trehalose (17)

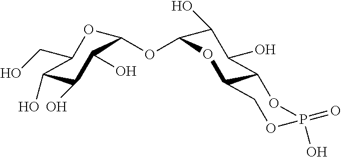

A solution of 67 (30 mg, 0.05 mmol, 1 eq) and ammonium hydroxide (28% in H₂O, 30 μL, 0.2 mmol, 4 eq) in water (4 mL) was stirred at RT for 14 hours after which TLC (1:1:1, water/isopropanol/ethyl acetate) showed the formation of a single product (R_{f} 0.3). The solvent and ammonium hydroxide were removed in vacuo. The crude solid was taken up in water (25 mL) and washed with DCM (3×10 mL). The aqueous layer was retained and the water removed under reduced pressure. The compounds were separated using silica gel chromatography (1 water:2 isopropanol:2 ethyl acetate). Lyophilization gave the title compound as an amorphous white solid (17 mg, 81%).

[α]_{D}¹⁹+52.6 (c=1.0 in H₂O); ¹H NMR (500 MHz, DEUTERIUM OXIDE) δ ppm 3.37 (1H, at, J_{3,4}=J_{4,5}=9.5 Hz, H-4′), 3.56 (1H, dd, J_{2,3}=10.1 Hz, J_{1,2}=3.8 Hz, H-2′), 3.67 (1H, dd, J_{2,3}=9.2 Hz, J_{1,2}=3.8 Hz, H-2), 3.68 (1H, at, J_{6a,6b}=J_{5,6b}=6.0 Hz, H-6′ b), 3.73-3.80 (3H, m, H-3′, H-5′, H-6′ a), 3.89 (1H, ddd, J_{4,5}=9.5 Hz, J_{5,6ax}=9.0 Hz, J_{5,6eq}=0.9 Hz, H-5), 3.92 (1H, at, J_{2,3}=J_{3,4}=9.1 Hz, H-3), 4.01 (1H, td, J_{5,6ax}=8.4 Hz, J_{6ax,6eq}=8.4 Hz, J_{P,6ax}=1.7 Hz, H-6ax), 4.02 (1H, at, J_{3,4}=J_{4,5}=9.5 Hz, H-4), 4.13 (1H, ddd, J_{P,6eq}=22.3 Hz, J_{6ax,6eq}=8.4 Hz, J_{5,6eq}=3.8 Hz, H-6 eq), 5.10 (1H, d, J_{1,2}=3.8 Hz, H-1′), 5.13 (1H, d, J_{1,2}=4.1 Hz, H-1); ¹³C NMR (126 MHz, DEUTERIUM OXIDE) δ ppm 60.4 (C-6′), 64.0 (d, J_{P,4}=4.8 Hz, C-4), 66.5 (d, J_{P,6}=5.7 Hz, C-6), 69.6 (C-4′), 70.4 (d, J_{P,3}=8.6 Hz, C-3), 70.8 (d, J_{P,2}=1.9 Hz, C-2), 71.0 (C-2′), 72.2 (C-3′), 72.5 (C-5′), 78.2 (d, J_{P,5}=4.8 Hz, C-5), 93.7 (C-1′), 94.0 (C-1); ³¹P(¹H) NMR (162 MHz, DEUTERIUM OXIDE) δ ppm −2.46; IR (KBr disc): ν=1139 (P═O), 3407 br (OH) cm⁻¹; MS (ESI⁻) m/z 403.5 (M−H⁺); HRMS (ESI⁻) m/z calcd. for C₁₂H₂₁O₁₃P (M−H⁺): 403.0647. Found: 403.0649.

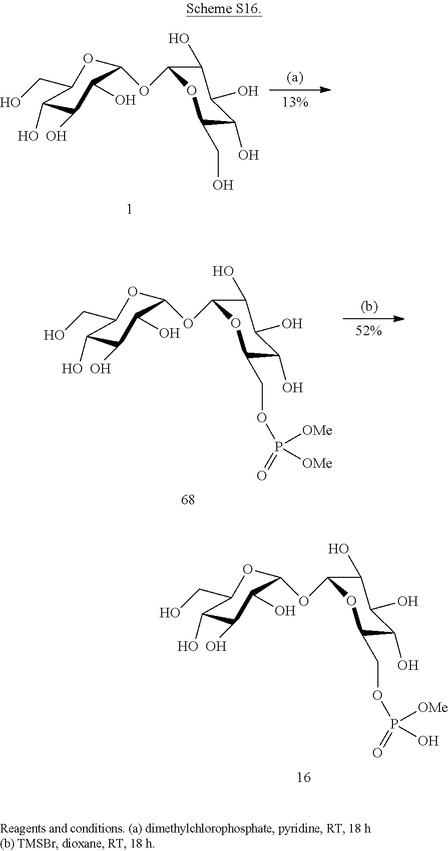

### 6-O-(dimethoxyphosphoryl)-D-trehalose (68)

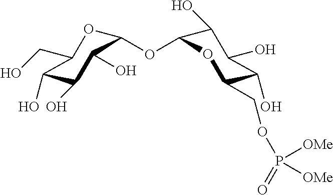

To a suspension of D-trehalose (0.25 g, 0.73 mmol, 1 eq) in anhydrous pyridine (10 mL) was added dropwise dimethylchlorophosphate (79 μL, 0.73 mmol, 1 eq). TLC (1 water:2 isopropanol:4 ethyl acetate) after 18 hours showed the presence of several products (desired product R_{f} 0.12). The reaction was quenched with methanol (5 mL). The reaction mixture was concentrated in vacuo, and the residue co-evaporated with toluene to remove pyridine. Silica gel chromatography (1:2:4, water/isopropanol/ethyl acetate) allowed isolation of the desired product as a white amorphous solid (43 mg, 13%).

[α]_{D}¹⁸+83.3 (c=1.0 in H₂O); ¹H NMR (500 MHz, DEUTERIUM OXIDE) δ ppm 3.36 (1H, at, J_{3,4}=J_{4,5}=9.5 Hz, H-4′), 3.43 (1H, dd, J_{3,4}=10.1 Hz, J_{4,5}=9.1 Hz, H-4), 3.56 (1H, dd, J_{2,3}=10.4 Hz, J_{1,2}=3.8 Hz, H-2′), 3.57 (1H, t, J_{2,3}=10.4 Hz, J_{1,2}=3.8 Hz, H-2), 3.67 (1H, dd, J_{6a,6b}=11.5 Hz, J_{5,6a}=5.0 Hz, H-6a′), 3.72 (1 H, m, H-3′), 3.75 (3H, d, J_{P,H}=11.0 Hz, OMe), 3.75-3.81 (3H, m, H-3, H-5′, H-6b′), 3.76 (3H, d, J_{P,H}=11.0 Hz, OMe), 3.91 (1H, dt, J_{4,5}=10.1 Hz, J_{5,6a}=J_{5,6b}=2.6 Hz, H-5), 4.25-4.29 (2H, m, H-6a, H-6b), 5.10 (1H, d, J_{1,2}=3.8 Hz, H-1′), 5.13 (1H, d, J_{1,2}=3.8 Hz, H-1); ¹³C NMR (126 MHz, DEUTERIUM OXIDE) δ ppm 55.1 (d, J_{P,C}=8.8 Hz, 2×OMe), 60.5 (C6′), 66.7 (d, J_{P,6}=5.7 Hz, C-6), 69.0 (C-4), 69.6 (C-4′), 70.5 (d, J_{P,5} 6.7 Hz, C-5), 70.9 (C-2), 71.0 (C-2′), 72.2 (C-5′), 72.4 (C-3), 72.5 (C-3′), 93.4 (C-1), 93.5 (C-1′); IR (KBr disc): ν=1260 (P═O), 3486 br (OH) cm⁻¹; MS (ESI⁺) m/z 451.1 (M+H⁺), 473.1 (M+Na⁺), MS (ESI⁻) m/z 449.6 (M−H⁺); HRMS (ESI⁺) calcd. for C₁₄H₂₇O₁₄P (M+Na): 473.1031. Found: 473.1027.

### 6-O-monomethoxyphosphoryl-D-trehalose (16)

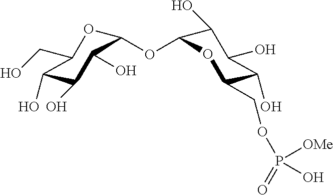

A suspension of 68 (11 mg, 25 μmol, 1 eq) in dioxane was briefly sonicated for 5 minutes. TMSBr (33 μL, 0.25 mmol, 10 eq) was added to this mixture at RT. The reaction was monitored by mass spectrometry (ESI⁻) and TLC (1 water:2 isopropanol:2 ethyl acetate) and after 3 hours, two deprotected analogues were detected (desired product R_{f} 0.1). The reaction was quenched by the addition of water (1 mL) and the solvents removed in vacuo. The crude mixture was taken up in water (3 mL) and washed with ethyl acetate (3×1 mL). The aqueous layer was concentrated in vacuo and the products were separated by HPLC through an Applied Biosystems, Poros® HQ strongly basic anion exchange column (10 mm×100 mm, 50 μm). A gradient from 0 mM to 500 mM aqueous NH₄HCO₃ was used as the mobile phase at a flow rate 20 mL/min and eluants were detected with an Evaporative Light Scattering (ELSD) detector. The title compound was not retained on the column and was immediately eluted whereas 15 was eluted at approximately 4.5 minutes (see previous chromatogram). Repeated lyophilization gave 16 (5.7 mg, 52%) and 15 (1.2 mg, 11%) as white amorphous solids.

[α]_{D}²⁵+38.8 (c=0.24 in H₂O); ¹H NMR (500 MHz, DEUTERIUM OXIDE) δ ppm 3.36 (1H, t, J_{3′,4′}=9.3 Hz, J_{4′,5′}=9.3 Hz, H-4′), 3.45 (1H, t, J_{3,4}=9.5 Hz, J_{4,5}=9.5 Hz, H-4), 3.52 (3H, d, J_{P,H}=11.0 Hz, OMe), 3.57 (1H, dd, J_{2′,3′}=8.3 Hz, J_{1′,2′}=3.3 Hz, H-2′), 3.59 (1H, dd, J_{2,3}=9.3 Hz, J_{1,2}=3.8 Hz, H-2), 3.68 (1H, dd, J_{6′a,6′b}=11.2 Hz, J_{5,6′b}=3.9 Hz, H-6′ b), 3.72 (1H, m, H-5′), 3.76 (2H, t, J_{2,3}=9.5 Hz, J_{3,4}=9.5 Hz, H-3, H-3′), 3.76 (1H, m, H-6′ a), 3.96 (1H, m, H-5), 4.01 (2H, m, H-6a, H-6b), 5.11 (1H, d, J_{1′,2′}=4.1 Hz, H-1′), 5.12 (1H, d, J_{1,2}=4.4 Hz, H-1); ¹³C NMR (126 MHz, DEUTERIUM OXIDE) δ ppm 52.9 (d, J_{P,C}=5.7 Hz, OMe), 60.5 (C-6′), 64.1 (d, J_{P,6}=1.9 Hz, C-6), 69.2 (C-4), 69.7 (C-4′), 70.9 (C-2), 71.0 (C-2′), 71.7 (C-5), 72.1 (C-5′), 72.4 (C-3), 72.4 (C-3′), 93.3 (C-1), 93.4 (C-1′); IR (KBr disc) ν=1137 (P═O), 3440 br (OH) cm⁻¹; (ESI⁻) m/z 435.1 (M−H⁺); HRMS (ESI⁻) calcd. for C₁₃H₂₅O₁₄P (M−H⁺): 435.0909. Found: 435.0923.

### Synthesis of Trehalose-6-Azide

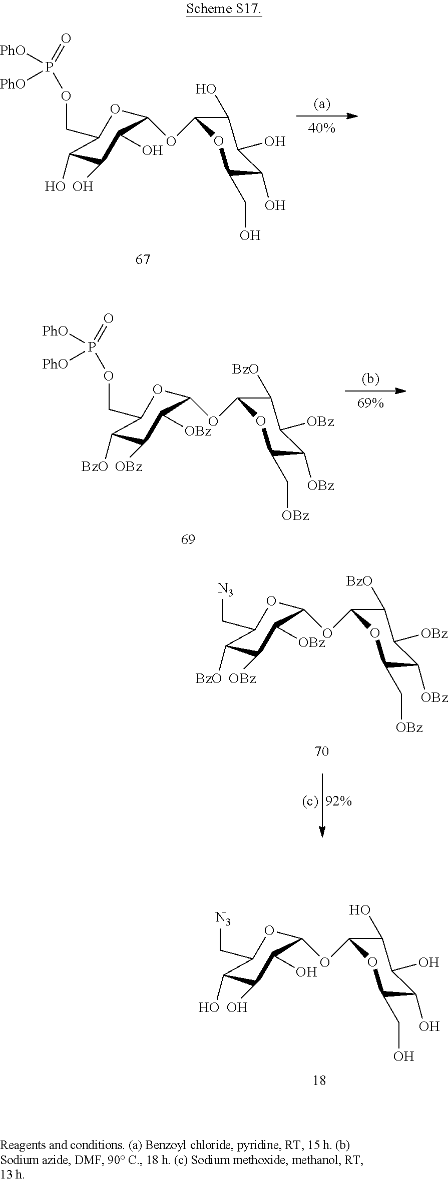

### 6-O-diphenoxyphosphoryl,2,2′,3,3′,4,4′,6′-O-benzoyl-D-trehalose (69)

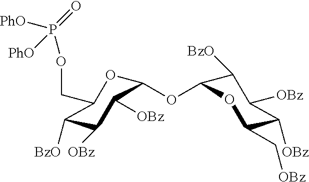

To a solution of 67 (100 mg, 0.18 mmol, 1 eq) in dry pyridine (5 mL) at RT was added dropwise benzoyl chloride (0.22 mL, 1.93 mmol, 11 eq). The reaction mixture was stirred for 15 hours after which TLC (1:1 petrol/ethyl) showed full consumption of starting material (R_{f} 0) and the formation of a single product (R_{f} 0.5). The reaction was quenched with methanol (1 mL) and the mixture was concentrated under reduced pressure. The resultant residue was partitioned between ethyl acetate (25 mL) and water (25 mL) and the aqueous layer was extracted with ethyl acetate (2×25 mL). The combined organics were washed with 1M HCl (3×25 mL), saturated NaHCO₃ (25 mL), brine (25 mL), dried over MgSO₄ and concentrated in vacuo. Silica gel chromatography (1:1 petrol/ethyl) yielded the target compound as a colourless oil (0.091 g, 40%).

[α]_{D}¹⁹+148.2 (c=0.46 in CHCl₃); ¹H NMR (400 MHz, CHLOROFORM-d) δ ppm 3.78 (1H, ddd, J_{6a,6b}=11.4 Hz, J_{P,6a}=6.9 Hz, J_{5,6a}=4.0 Hz, H-6a), 3.87 (1H, m, H-6b), 3.91 (1H, dd, J_{6a,6b}=12.3 Hz, J_{5,6a}=4.6 Hz, H-6′ a), 4.03 (1H, dd, J_{6a,6b}=12.6 Hz, J_{5,6b}=2.7 Hz, H-6′ b), 4.14 (1H, m, H-5), 4.28 (1H, ddd, J_{4,5}=10.1 Hz, J_{5,6a}=4.3 Hz, J_{5,6b}=2.9 Hz, H-5′), 5.31 (1H, dd, J_{2,3}=10.2 Hz, J_{1,2}=3.8 Hz, H-2), 5.48 (1H, dd, J_{2,3}=10.2 Hz, J_{1,2}=3.8 Hz, H-2′), 5.58 (1H, at, J_{3,4}=J_{4,5}=9.9 Hz, H-4), 5.62 (1H, d, J_{1,2}=3.8 Hz, H-1), 5.65 (1H, d, J_{1,2}=3.8 Hz, H-1′), 5.69 (1H, at, J_{3,4}=J_{4,5}=9.9 Hz, H-4′), 6.24 (1H, at, J_{2,3}=J_{3,4}=9.5 Hz, H-3), 6.29 (1H, at, J_{2,3}=J_{3,4}=9.9 Hz, H-3′), 7.12-7.60 (31H, m, Ar—H), 7.80, 7.81, 8.08 (3×2 H, 3×d, Jₒᵣₜₕₒ₋ₘₑₜₐ=7.2 Hz, Bz Ar—Hₒᵣₜₕₒ), 7.94 (4H, d, Jₒᵣₜₕₒ₋ₘₑₜₐ=8.2 Hz, Bz Ar—Hₒᵣₜₕₒ), 8.00, 8.12 (2×2 H, 2×d, Jₒᵣₜₕₒ₋ₘₑₜₐ=7.3 Hz, Bz Ar—Hₒᵣₜₕₒ); ¹³C NMR (101 MHz, CHLOROFORM-d) δ ppm 61.9 (C-6′), 65.6 (d, J_{P,6}=6.4 Hz, C-6), 67.7 (C-4′), 68.5 (C-4), 68.6 (C-5′), 68.7 (d, J_{P,5}=6.4 Hz, C-5), 70.3 (2C, C-3, C-3′), 71.0 (C-2′), 71.2 (C-2), 92.4 (C-1′), 92.4 (C-1), 120.1 (d, J_{P,C}=4.8 Hz, P—OPh Ar—Cₒᵣₜₕₒ), 125.4 (d, J_{P,C}=7.2 Hz, P—OPh Ar—Cₚₐᵣₐ), 128.4-129.9 (m, ArC), 133.2, 133.3, 133.3, 133.5, 133.6, 133.7, 133.8 (7×Bz Ar—Cₚₐᵣₐ), 150.3 (d, J_{P,C}=7.2 Hz, P—OPh Ar—Cᵢₚₛₒ), 150.4 (d, J_{P,C}=6.4 Hz, P—OPh Ar—Cᵢₚₛₒ), 164.6, 164.9, 165.2, 165.2, 165.6, 165.6, 165.9 (7×C═O); IR (thin film) ν 1640 (C═O) cm⁻¹; (ESI) m/z 1320.4 (M+NH₄⁺), 1325.3 (M+Na⁺); Isotopic distribution: species observed (M+Na⁺), peaks observed 1325.27 (100%), 1326.28 (74%), 1327.28 (29%), 1328.29 (8%), 1329.29 (2%) peaks calculated 1325.32 (100%), 1326.32 (80%), 1327.32 (36%), 1328.32 (12%), 1329.33 (3%).

### 6-Azido-2,2′,3,3′,4,4′,6′-O-benzoyl-D-trehalose (70)

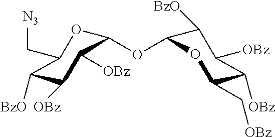

A solution of 69 (90 mg, 0.069 mmol, 1 equi) in DMF (3 mL) was heated to 90° C. with sodium azide (9 mg, 0.14 mmol, 2 equi). The reaction mixture was maintained at this temperature with aggressive stirring for 18 hours after which TLC (1:1 petrol/ethyl acetate) showed complete consumption of starting material (R_{f} 0.5) and the formation of a product (R_{f} 0.4). The reaction mixture was concentrated in vacuo and the residue partitioned between ethyl acetate (30 mL) and water (20 mL). The aqueous layer was extracted with ethyl acetate (2×20 mL) and the combined organics washed with brine (3×25 mL), dried over MgSO₄ and the solvent removed in vacuo. The compound was purified by silica gel chromatography (3:2 petrol/ethyl acetate) to afford the desired compound as a colourless oil (52 mg, 69%).

[α]_{D}¹⁷+210.4 (c=1.0 in CHCl₃); ¹H NMR (400 MHz, CHLOROFORM-d) δ ppm 2.89-2.91 (2H, m, H-6a, H-6b), 3.88 (1H, dd, J_{6a,6b}=12.5 Hz, J_{5,6a}=4.7 Hz, H-6′ a), 4.03 (1H, dd, J_{6a,6b}=12.6 Hz, J_{5,6b}=2.5 Hz, H-6′ b), 4.14 (1H, ddd, J_{4,5}=9.8 Hz, J_{5,6a}=7.6 Hz, J_{5,6b}=3.0 Hz, H-5), 4.32 (1H, ddd, J_{4,5}=10.4 Hz, J_{5,6a}=4.3 Hz, J_{5,6b}=2.8 Hz, H-5′), 5.50 (2H, dd, J_{2,3}=J_{2,3}=10.1 Hz, J_{1,2}=J_{1,2}=3.8 Hz, H-2, H-2′), 5.57 (1H, at, J_{3,4}=J_{4,5}=9.9 Hz, H-4), 5.70 (1H, at, J_{3,4}=J_{4,5}=10.0 Hz, H-4′), 5.76 (1H, d, J_{1,2}=4.0 Hz, H-1), 5.78 (1H, d, J_{1,2}=4.0 Hz, H-1′), 6.26 (1H, at, J_{2,3}=J_{3,4}=9.9 Hz, H-4), 6.30 (1H, at, J_{2,3}=J_{3,4}=10.0 Hz, H-3′), 7.32-7.60 (21H, m, Ar—H), 7.83, 7.85 (2×2 H, 2×dd, Jₒᵣₜₕₒ₋ₘₑₜₐ=8.2 Hz, Jₒᵣₜₕₒ₋ₒᵣₜₕₒ=1.3 Hz, Ar—Hₒᵣₜₕₒ), 7.93, 7.94 (2×2 H, 2×dd, Jₒᵣₜₕₒ₋ₘₑₜₐ=8.7 Hz, Jₒᵣₜₕₒ₋ₒᵣₜₕₒ=1.5 Hz, Ar—Hₒᵣₜₕₒ), 7.96 (2H, dd, Jₒᵣₜₕₒ₋ₘₑₜₐ=8.2 Hz, Jₒᵣₜₕₒ₋ₒᵣₜₕₒ=1.0 Hz, Ar—Hₒᵣₜₕₒ), 8.08, 8.14 (2×2 H, 2×dd, Jₒᵣₜₕₒ₋ₘₑₜₐ=7.4 Hz, Jₒᵣₜₕₒ₋ₒᵣₜₕₒ=1.3 Hz, Ar—Hₒᵣₜₕₒ); ¹³C NMR (101 MHz, CHLOROFORM-d) δ ppm 50.0 (C-6), 61.9 (C-6′), 68.5 (C-5′), 68.6 (C-4′), 69.1 (C-4), 69.7 (C-5), 70.1 (C-3′), 70.2 (C-3), 71.1 (C-2′), 71.3 (C-2), 92.6 (C-1′), 92.7 (C-1), 128.4, 128.4, 128.7, 128.7 (4× as, Ar—Cₘₑₜₐ), 128.5, 128.7, 128.7, 128.8 129.0, 129.0, 129.4 (Ar—Cᵢₚₛₒ), 129.8, 129.9, 129.9, 129.9 (4× as, Ar—Cₒᵣₜₕₒ), 133.1, 133.3, 133.3, 133.5, 133.6, 133.7, 133.8 (Ar—Cₚₐᵣₐ), 164.9, 164.9, 165.2, 165.3, 165.5, 165.6, 165.8 (C═O); IR (thin film) ν=1732 (C═O), 2107 (N₃) cm⁻¹; (ESI⁺) m/z 1113.4 (M+NH₄¹), 1118.4 (M+Na⁺); Isotopic distribution: peaks observed: (M+Na⁺) peaks 1118.30 (100%), 11190.31 (66%), 1120.31 (24%), 1121.31 (7%), 1122.32 (2%) peaks calculated 1118.30 (100%), 1119.30 (68%), 1120.30 (26%), 1121.30 (7%), 1122.31 (2%).

### 6-Azido-D-trehalose (18)²⁴

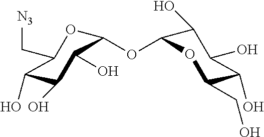

To a stirring solution of 70 (52 mg, 0.047 mmol, 1 equi) in methanol (3 mL) was added sodium methoxide (2.5 mg, 0.047 mmol, 1 equi). The reaction mixture was stirred for 13 hours at which point TLC (1:4:4 water/isopropanol/ethyl acetate) indicated complete consumption of the starting material (R_{f} 0) and the formation of a single product (R_{f} 0.3). The reaction was quenched with Dowex 50WX8 100-200 mesh (H⁺ form), filtered and the solvent removed in vacuo. The compound was purified by silica gel chromatography (1:4:4 water/isopropanol/ethyl acetate). Lyophilization gave the title compound as a white amorphous solid (16 mg, 92%).

[α]_{D}²⁵+167.3 (c=0.26 in H₂O) [Lit. [α]_{D}+149 (c=0.81 in MeOH)]²⁴; ¹H NMR (500 MHz, DEUTERIUM OXIDE) δ ppm 3.35 (1H, at, J_{3,4}=J_{4,5}=9.3 Hz, H-4′), 3.37 (1H, at, J_{3,4}=J_{4,5}=9.5, H-4), 3.47 (1H, dd, J_{6a,6b}=13.6 Hz, J_{5,6a}=5.7 Hz, H-6a′), 3.56 (1H, dd, J_{2,3}=10.4 Hz, J_{1,2}=3.8 Hz, H-2′), 3.58 (1H, dd, J_{2,3}=10.1 Hz, J₁₂=4.0 Hz, H-2), 3.59 (1H, dd, J_{6a,6b}=13.7 Hz, J_{5,6b}=2.5 Hz, H-6b′), 3.67 (1H, dd, J_{6a,6b}=11.7 Hz, J_{5,6a}=5.0 Hz, H-6a), 3.72-3.79 (4H, m, H-5, H-3,H-3′, H-6b), 3.88 (1H, ddd, J_{4,5}=10.1 Hz, J_{5,6a}=3.8 Hz, J_{5,6b}=2.2 Hz, H-5′), 5.10 (1H, d, J_{1,2}=4.2 Hz, H-1), 5.11 (1H, d, J_{1,2}=4.7 Hz, H-1′); ¹³C NMR (126 MHz, DEUTERIUM OXIDE) δ ppm 50.8 (C-6′-azide), 60.4 (C-6), 69.6 (C-4′), 70.4 (C-4), 70.9 (C-2′), 70.9 (C-2), 70.9 (C-5), 72.2 (C-5′), 72.3 (C-3′), 72.5 (C-3), 93.4 (C-1′), 93.6 (C-1); IR (KBr disc) ν=2110 (N₃), 3456 br (OH); (ESI⁺) m/z 390.1 (M+Na⁺).

### Synthesis of Symmetric Trehalose analogues: dehydrative glycosylations

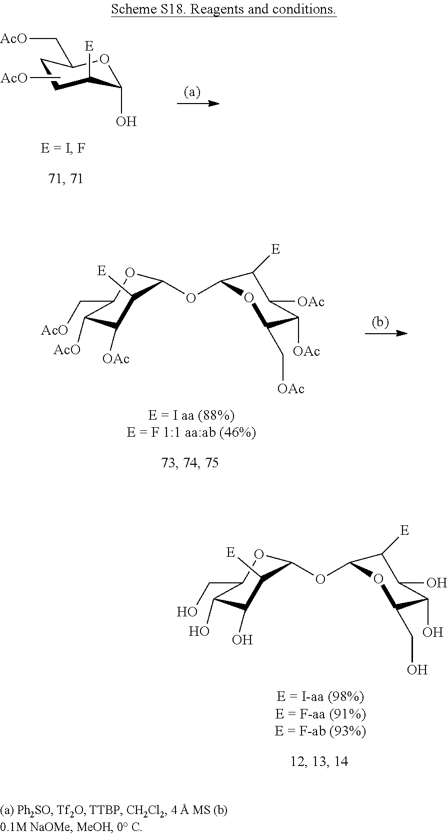

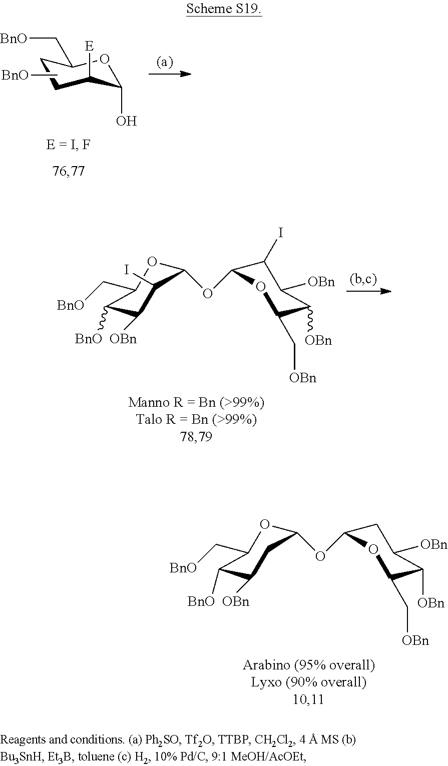

Precursors 73,74,75,78 and 79 were prepared as described in the literature²⁵ and their synthesis will be reported in due course.

### 2′-Deoxy-α-D-arabino-hexopyranosyl-(1→1)-2-deoxy-α-D-arabino-hexopyranoside (10)²⁶

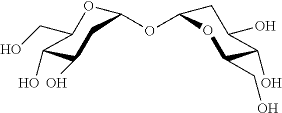

A solution of 78²⁵ (100 mg, 0.091 mmol, 1 eq), Bu₃SnH (151 μL, 0.544 mmol, 6 eq), and 1 M Et₃B in hexane (54 μL, 0.054 mmol, 0.6 eq) in dry toluene (910 μL) was stirred at room temperature for 5 h. The reaction was monitored by TLC (3:1 petrol/ethyl acetate) and upon completion (R_{f} 0.22) was filtered through a short path of silica and concentrated under reduced pressure. The crude yellowish syrup was dissolved in 9:1 methanol/ethyl acetate (9.1 mL) at room temperature and subjected to hydrogenolysis (1 atm) using 10% Pd/C (725 mg). After stirring at the same temperature for 10 h, TLC (7:2:1 ethyl acetate/methanol/water) analysis indicated the presence of a highly polar compound (R_{f} 0.20). The reaction mixture was filtered through Celite® and concentrated under reduced pressure. The residue was purified by column chromatography (7:2 ethyl acetate/methanol) to afford the titled compound (26.7 mg, 95% over two steps) as a white fluffy solid.

¹H NMR (400 MHz, DEUTERIUM OXIDE) δ ppm 1.64 (2H, ddd, J_{2az,2eq}=13.2 Hz, J_{2ax,3}=12.5 Hz, J_{1,2ax}=3.3 Hz, H-2ax, H-2ax′), 2.07 (2H, ddd, J_{2az,2eq}=13.2 Hz, J_{2eq,3}=5.1 Hz, J_{1,2eq}=1 Hz, H-2 eq, H-2 eq′), 3.28 (2H, dd, J_{3,4}=J_{4,5}=9.5 Hz, H-4, H-4′), 3.55 (2H, ddd, J_{4,5}=9.5 Hz, J_{5,6b}=2.2 Hz, J_{5,6a}=5.5 Hz, H-5, H-5′), 3.66 (2H, dd, J_{6a,6b}=12.1 Hz, J_{5,6a}=5.5 Hz, H-6a, H-6a′), 3.75 (2H, dd, J_{6a,6b}=12.1 Hz, J_{5,6b}=2.2 Hz, H-6b, H-6b′), 3.88 (2H, ddd, J_{2ax,3}=12.5 Hz, J_{3,4}=9.5 Hz, J_{2eq,3}=5.1 Hz, H-3, H-3′), 5.15 (2H, d, J_{1,2ax}=3.3 Hz, H-1, H-1′); ¹³C NMR (101 MHz, DEUTERIUM OXIDE) δ ppm 37.0 (2C, C-2, C-2′), 61.3 (2C, C-6, C-6′), 68.8 (2 C. C-3, C-3′), 71.6 (2C, C-4, C-4′), 73.4 (2C, C-5, C-5′), 93.1 (2C, C-1, C-1′); MS (ESI⁺) m/z 333.1 (M+Na⁺). HRMS (ESI⁺) calcd. for C₁₂H₂₂NaO₉ (M+Na⁺): 333.1162. Found: 333.1156; Spectroscopic data are in agreement with those reported²⁶.

### 2′-Deoxy-α-D-lyxo-hexopyranosyl-(1→1)-2-deoxy-α-D-lyxo-hexopyranoside (11)

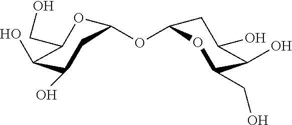

A solution of 79²⁵ (200 mg, 0.18 mmol, 1 eq), Bu₃SnH (302 μL, 1.09 mmol, 6 eq), and 1M Et₃B in hexane (109 μL, 0.11 mmol, 0.6 eq) in dry toluene (1.8 mL) was stirred at room temperature for 24 h. The reaction was monitored by TLC (3:1 petrol/ethyl acetate) and upon completion (R_{f} 0.38) was filtered through a short path of silica and concentrated under reduced pressure. The crude yellowish syrup was dissolved in 9:1 methanol/ethyl acetate (18.1 mL) at room temperature and subjected to hydrogenolysis (1 atm) using 10% Pd/C (1.4 g). After stirring at the same temperature for 22 h, TLC (7:2:1 ethyl acetate/methanol/water) analysis indicated the presence of a highly polar compound (R_{f} 0.15). The reaction mixture was filtered through Celite® and concentrated under reduced pressure. The residue was purified by column chromatography (7:2 ethyl acetate/methanol) to afford the titled compound (50.7 mg, 90% over two steps) as a white fluffy solid.

[α]_{D}²¹+151.8 (c=0.40 in MeOH); ¹H NMR (400 MHz, DEUTERIUM OXIDE) δ ppm 1.79 (2H, ddd, J_{2az,2eq}=13.2 Hz, J_{2ax,3}=11.7 Hz, J_{1,2ax}=4.4 Hz, H-2eq, H-2 eq′), 3.68-3.59 (2H, m, H-6a, H-6a′, H-6b, H-6b′), 3.76 (2H, d, J_{3,4}=2.7 Hz, H-4, H-4′), 3.81 (2H, dd, J_{5,6b}=4.8 Hz, J_{5,6a}=7.3 Hz, H-5, H-5′), 4.02 (2H, ddd, J_{2ax,3}=11.7 Hz, J_{2eq,3}=4.1 Hz, J_{3,4}=2.7 Hz, H-3, H-3′), 5.19 (2H, d, J_{1,2ax}=4.4 Hz, H-1, H-1′); ¹³C NMR (101 MHz, DEUTERIUM OXIDE) δ ppm 31.3 (2C, C-2, C-2′), 62.1 (2C, C-6, C-6′), 65.1 (2C, C-3, C-3′), 68.0 (2C, C-4, C-4′), 71.8 (2C, C-5, C-5′), 92.8 (2C, C-1, C-1′); MS m/z (ESI) 333.1 (M+Na⁺); HRMS (ESI) calcd. for C₁₂H₂₂NaO₉ (M+Na): 333.1162. Found: 333.1156.

### 2′-Deoxy-2′-iodo-α-D-mannopyranosyl-(1→1)-2-deoxy-2-iodo-α-D-mannopyranoside (12)

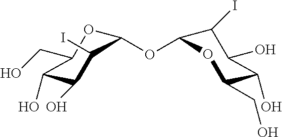

73 (20 mg, 0.025 mmol) was treated with 0.1M NaOMe in methanol (175 μL) at 0° C. The reaction mixture was stirred at the same temperature for 15 minutes and neutralized with DOWEX 50WX8 (H⁺ form) cation exchange resin. The resin was filtered off and washed with methanol. TLC (7:1 ethyl acetate/methanol) analysis indicated the presence of a polar compound (R_{f} 0.30). The resulting solution was concentrated under reduced pressure to afford XX (13.5 mg, 98%) as a white fluffy solid.

[α]_{D}²¹+97.1 (c=0.7 in MeOH); ¹H NMR (METHANOL-d₄, 500 MHz) δ ppm 3.10 (2H, dd, J_{3,4}=8.5 Hz, J_{2,3}=4.1 Hz, H-3, H-3′), 3.61 (2H, dd, J_{3,4}=J_{4,5}=8.5 Hz, H-4, H-4′), 3.69-3.65 (4H, m, H-5, H-6b, H-5′, H-6b′), 3.84 (2H, dd, J_{6a,6b}=14.1 Hz, J_{5,6a}=5.3 Hz, H-6a, H-6a′), 4.44 (2H, d, J_{2,3}=4.1 Hz, H-2, H-2′), 5.48 (1 H, s, H-1); ¹³C NMR (METHANOL-d₄, 125.8 MHz) δ ppm 37.2 (2C, C-2, C-2′), 63.0 (2C, C-6, C-6′), 69.8 (2C, C-3, C-3′), 70.9 (2C, C-4, C-4′), 76.7 (2C, C-5, C-5′), 99.3 (2C, C-1, C-1′); IR (KBr): ν=3356, 2924, 1594, 1436 cm⁻¹; MS m/z (ESI 584.9 (M+Na⁺); HRMS (ESI) calcd. for C₁₂H₂₀I₂NaO₉ (M+Na⁺): 584.9094. Found: 584.9089.

### 2′-Deoxy-2′-fluoro-α-D-mannopyranosyl-(1→1)-2-deoxy-2-fluoro-α-D-mannopyranoside (13)

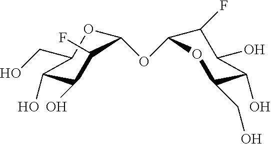

74 (20 mg, 0.033 mmol, 1 eq) was treated with 0.1M NaOMe in methanol (234 μL, 0.02 mmol, 0.6 equi) at 0° C. The reaction mixture was stirred at the same temperature for 15 minutes and neutralized with DOWEX, 50WX8 (H⁺ form) cation exchange resin. The resin was filtered off and washed with methanol. TLC (7:1 ethyl acetate/methanol) analysis indicated the presence of a polar compound (R_{f} 0.27). The resulting solution was concentrated under reduced pressure to afford the titled compound (10.6 mg, 91%) as a white fluffy solid.

[α]_{D}²¹+109.9 (c=0.16 in MeOH); ¹H NMR (METHANOL-d₄, 500 MHz) δ ppm 3.57-3.77 (8H, m, H-3, H-3′, H-4, H-4′, H-5, H-5′, H-6b, H-6b′), 3.76 (2H, d, J_{6a,6b}=11.7 Hz, H-6a, H-6a′), 4.64 (2H, d, J_{2,F}=50.1 Hz, H-2, H-2′), 5.34 (2H, d, J_{1,F}=7.6 Hz, H-1, H-1′); ¹³C NMR (METHANOL-d₄, 126 MHz) δ ppm 62.8 (2C, C-6, C-6′), 68.7 (2C, C-4, C-4′), 71.6 (2C, d, J_{3,F}=13.4 Hz, C-3, C-3′), 76.0 (2C, C-5, C-5′), 91.0 (2C, d, J_{2,F}=175.5 Hz, C-2, C-2′), 94.4 (2C, d, J_{1,F}=30.5 Hz, C-1, C-1′); ¹⁹F NMR (METHANOL-d₄, 470 MHz) δ ppm −206.5 (ddd, J_{2,F}=50.1 Hz, J_{3,F}=30.5 Hz, J_{1,F}=7.6 Hz); IR (KBr): ν=3355, 2926, 2855, 1417, 1066 cm⁻¹; MS m/z (ESI⁺) 369.1 (M+Na⁺); MS m/z (ESI⁺) 369.1 (M+Na⁺); HRMS (ESI⁺) calcd. for C₁₂H₂₀F₂NaO₉ (M+Na⁺): 369.0973. Found: 369.0968.

### 2′-Deoxy-2′-fluoro-α-D-mannopyranosyl-(1→1)-2-deoxy-2-fluoro-β-D-mannopyranoside (14)

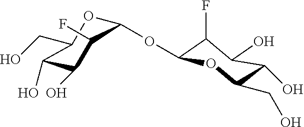

75 (12.7 mg, 0.021 mmol, 1 eq) was treated with 0.1M NaOMe in methanol (149 μL, 0.014 mmol, 0.7 eq) at 0° C. The reaction mixture was stirred at the same temperature for 15 minutes and neutralized with DOWEX, 50WX8 (H⁺ form) cation exchange resin. The resin was filtered off and washed with methanol. TLC (7:1 ethyl acetate/methanol) analysis indicated the presence of a polar compound (R_{f} 0.11). The resulting solution was concentrated under reduced pressure to afford the titled compound (6.8 mg, 93%) as a white fluffy solid.

[α]_{D}²¹+62.7 (c=0.11 in MeOH); ¹H NMR (METHANOL-d₄, 500 MHz) δ ppm 3.31 (1H, overlapped, H-5′), 3.70-3.46 (5H, m, H-3, H-4, H-6b, H-4′,H-6b′), 3.80 (1H, dd, J_{3,F}=31.0 Hz, J_{3,4}=12.3 Hz, J_{2,3}=2.5 Hz, H-3′), 3.89 (1H, d, J_{6a,6b}=12.9 Hz, H-6a), 3.91 (1H, d, J_{6a,6b}=11.7 Hz, H-6a′), 3.98 (1H, dd, J_{4,5}=J_{5,6a}=8.8 Hz, H-5), 4.65 (1H, dd, J_{2,F}=49.3 Hz, J_{2′,3′}=2.5 Hz, H-2′), 4.69 (1H, dd, J_{2,F}=53.9 Hz, J_{2,3}=2.2 Hz, H-2), 4.86 (1H, overlapped, H-1), 5.28 (1H, d, J_{1′,F′}=7.1 Hz, H-1′); ¹³C NMR (METHANOL-4, 125.8 MHz) δ ppm 63.2 (C-6′), 63.3 (C-6), 68.8 (C-4′), 69.1 (C-4), 71.6 (d, J_{3,F}=18.1 Hz, C-3′), 71.6 (d, J_{3,F}=18.1 Hz, C-3), 75.5 (C-5), 79.0 (C-5′), 90.9 (1C, d, J_{2,F}=174.5 Hz, C-2′), 91.9 (1C, d, J_{2,F}=184.1 Hz, C-2), 99.7 (1C, d, J_{1,F}=30.5 Hz, C-1′), 99.9 (1C, d, J_{1,F}=15.3 Hz, C-1); ¹⁹F NMR (METHANOL-d₄, 377 MHz) δ ppm −206.9 (ddd, J_{2′,F′}=49.3 Hz, J_{3′,F′}=31.0 Hz, J_{1′,F′}=7.1 Hz, F-2′ α), −222.9 (ddd, J_{2,F}=53.9 Hz, J_{3,F}=29.8 Hz, J_{1,F}=19.5 Hz, F-2β); IR (KBr): ν=3355, 2926, 2855, 1417, 1066 cm⁻¹; MS m/z (EST) 369.1 (M+Na); HRMS (ESI⁺) calcd. for C₁₂H₂₀F₂NaO₉ (M+Na⁺): 369.0973. Found: 369.0968.

### Enzymatic Synthesis of 2-F-Trehalose-6P

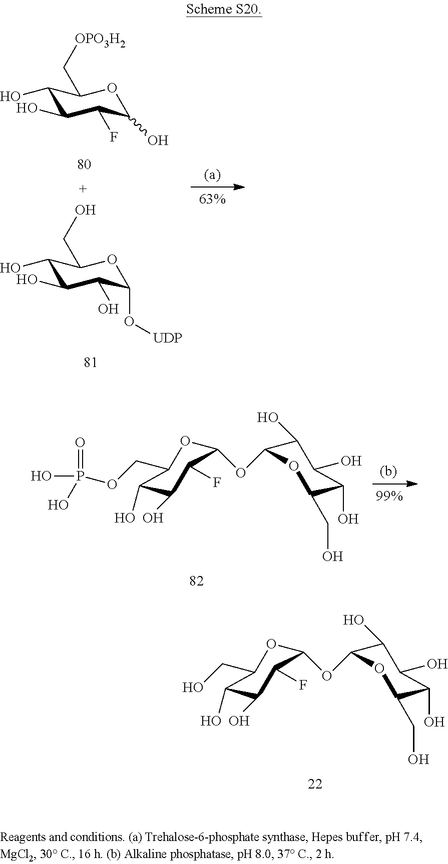

### α-D-Glucopyranosyl 2-deoxy-2-fluoro-α-D-glucopyranose-6-phosphate (82)

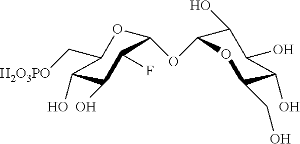

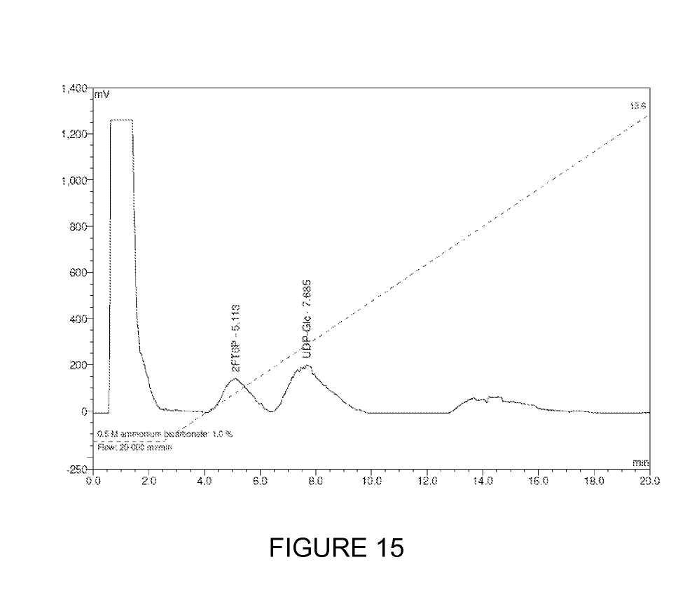

80 was prepared by the known procedure²⁷ was dissolved in 0.2 M, pH 7.4 Hepes buffer containing 10 mM MgCl₂ in the presence of 95 mg of 81. To this mixture added trehalose-6-phosphate synthase cloned and expressed from E. coli²⁸ to a final concentration of 21.6 μM and pH was adjusted to 7.4. Total volume was 3 mL. The reaction mixture was incubated at 30° C. overnight. After confirming the reaction was completed by electrospray ionization mass spectrometer (ESI MS), the mixture was filtered using centrifugal concentrator Viva spin 500 (Sartorius) of nominal molecular weight cutoff 10,000 to remove proteins. Typically, overnight reaction was conducted to confirm the completion of the reaction. The filtrate was divided into three volumes and purified by the HPLC (Dionex Ultimate 3000) using a strong anion exchange column Applied Biosystems, Poros® HQ (10 mm×100 mm, 50 μm). The HPLC was eluted with gradient of 0-500 mM ammonium bicarbonate at a flow rate of 20 mL/min and eluants were detected with an evaporative light scattering detector (ELSD). The product was eluted at retention time of 5.11 minutes, as shown in FIG. 15. Collected fractions were pooled and concentrated under reduced pressure, yielding a white solid (25 mg, 63%).

¹H NMR (400 MHz, DEUTERIUM OXIDE) δ ppm 3.29 (1H, at, J_{3,4}=J_{4,5}=9.55 Hz, H-4′), 3.52 (1H, dd, J_{2,3}=10.0 Hz, J_{1,2}=3.8 Hz, H-2′), 3.55 (1H, at, J_{3,4}=J_{4,5}=9.85 Hz, H-4) 3.61-3.90 (7H, m, H-5, H-6a, H-6b, H-3′, H-5′, H-6a′, H-6b′), 3.98 (1H, dt, J_{3,F}=13.1 Hz, J_{2,3}=J_{3,4}=9.3 Hz, H-3), 4.41 (1H, ddd, J_{2,F}=49.0 Hz, J_{1,2}=4.2, J_{2,3}=9.5 Hz, H-2), 5.09 (1H, d, J_{1,2}=3.8, H-1′), 5.28 (d, 1H, J_{1,2}=4.2 Hz, H-1), ¹³C NMR (126 MHz, DEUTERIUM OXIDE) δ ppm 60.5 (C-6′), 62.2 (1C, d, J_{C-6,P}=3.8 Hz, C-6), 68.4 (C-4), 69.5 (C-4′), 70.7 (1C, d, J_{C-3,F}=16.1 Hz, C-3), 70.9 (C-2′), 71.9 (1C, d, J_{C-5,P}=6.7 Hz, C-5), 72.2 (C-5′), 72.5 (C-3′), 89.6 (d, J_{C-2,F}=187.9 Hz, C-2), 91.4 (1C, d, J_{C-2,F}=22.2 Hz, C-1), 94.0 (C-1′); ¹⁹F(¹H) NMR (376 MHz, DEUTERIUM OXIDE) δ ppm −201.2, ³¹P(¹H) NMR (162 MHz, DEUTERIUM OXIDE) δ ppm 4.5. MS m/z (ESI⁻) 423.1 (M−H⁺). HRMS (ESI⁻): calcd. for C₁₂H₂₁FO₁₃P (M−H⁺) 423.0709. Found 423.0709.

### α-D-Glucopyranosyl 2-deoxy-2-fluoro-α-D-glucopyranose (22)

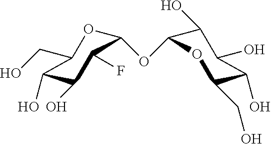

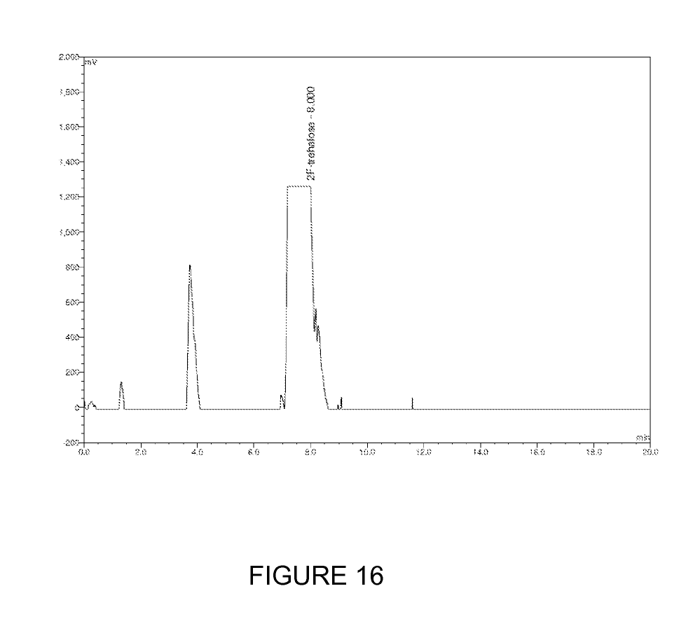

5 mg (12 μmol) of 82 (2-fluoro-trehalose-6-phosphate) was dissolved in water and pH was adjusted to 8.0 with 1M NaOH. 3 Units of alkaline phosphatase (Sigma) was added to initiate the reaction. The reaction mixture was incubated at 37° C. and the reaction progress was monitored by ESI MS. Typically, reaction was completed within 2 hours. After confirming the reaction was completed, the mixture was filtered using the centrifugal concentrator Viva spin 500 (Sartorius) of nominal molecular weight cutoff 10,000 to remove proteins. The filtrate was loaded onto a column packed with anion exchange resin DEAE cellulose (Sigma) and the fraction eluted with water was collected and concentrated under reduced pressure. The resulting syrup was loaded onto the Phenomenex Luna NH2 HPLC column (250×21.2 mm, 5 μam) on Dionex UltiMate 3000 system. Eluants were detected with an evaporative light scattering detector (ELSD). The product was eluted at 7.8 minutes of rentention time by an isocratic elution with 30/70 water/acetonitrile at a flow rate of 18.0 mL/min, as shown in FIG. 16. Fractions containing the product was pooled and concentrated under reduced pressure, yielding a colorless wax (4.0 mg, 99%).

¹H NMR (400 MHz, DEUTERIUM OXIDE) δ ppm 3.36 (1H, at, J_{3,4}=J_{4,5}=9.0 Hz, H-4′), 3.42 (1H, at, J_{3,4}=J_{4,5}=9.7 Hz, H-4), 3.56 (1H, dd, J_{1,2}=3.7 Hz, J_{2,3}=9.9 Hz, H-2′), 3.58-3.81 (7H, m, H-5, H-6a, H-6b, H-3′, H-5′, H-6a′, H-6b′), 3.97 (1H, dt, J_{3,F}=13.2 Hz, J_{2,3}=J_{3,4}=9.6 Hz, H-3), 4.36 (1H, ddd, J_{2,F}=48.7 Hz, J_{1,2}=4.0 Hz, J_{2,3}=9.6 Hz, H-2), 5.07 (1H, d, J_{1,2}=3.7 Hz, H-1′), 5.29 (1H, d, J_{1,2}=4.0 Hz, H-1); ¹³C NMR (126 MHz, DEUTERIUM OXIDE) δ ppm 60.3 (C-6 or C-6′), 60.5 (C-6 or C-6′), 69.0 (C-4), 69.1 (C-4′), 69.6 (C-3′), 70.9 (C-2′), 72.2 (C-5 or C-5′), 72.6 (C5 or C-5′), 71.1 (d, J_{C-3,F}=15.8 Hz, C-3), 89.5 (d, J_{C-2,F}=188.1 Hz, C-2), 91.2 (d, J_{F2,C1}=22.0 Hz, C-1), 94.0 (C-1′); ¹⁹F(¹H) NMR (376 MHz, DEUTERIUM OXIDE), δ ppm −201.2; MS m/z (ESI⁻) 379.1 (M+Cl⁻); HRMS (ESI⁻): calcd. for C₁₂H₂₀FO₁₀ (M−H⁺) 343.1035. Found: 343.1040.

### 4 and 6 Modified Trehalose

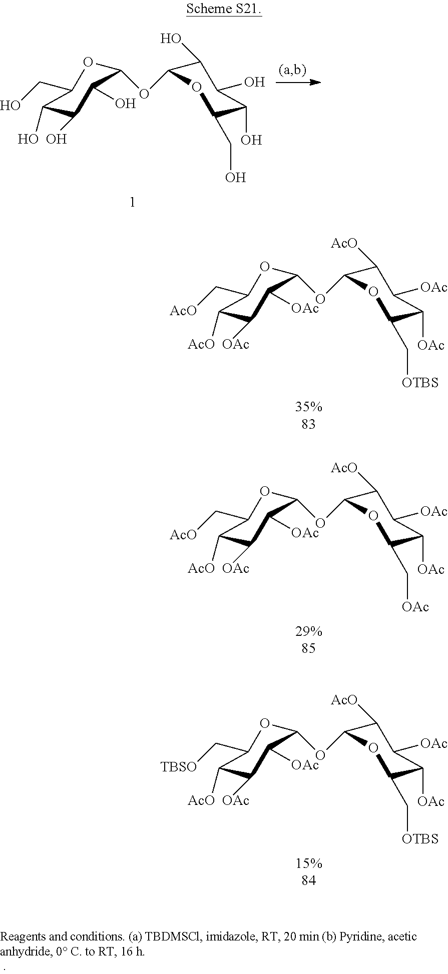

### 6-Tert-butyldimethylsilyl-2,3,4-tri-O-acetyl-α-D-glucopyranosyl 2,3,4,6-tetra-O-acetyl-α-D-glucopyranoside (83)

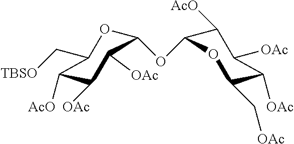

Anhydrous trehalose (3.42 g, 10.0 mmol, 1 eq) was added to 150 mL of dry DMF at 50° C. The solution was cooled down to room temperature and imidazole (1.36 g, 20.0 mmol, 2 eq) and TBDMSC1 (1.66 g, 11 mmol, 1.1 eq) were added. After 20 minutes, DMF was evaporated under high vacuum to give 8.53 g of yellow oil. This oil was dissolved in pyridine (100 mL) and DMAP (122 mg, 1.0 mmol, 0.1 eq) was added then the mixture was cooled to 0° C. and acetic anhydride (9.27 mL) was added slowly. The solution was stirred overnight at room temperature, upon which time TLC (4:1 chlorofom/ethyl acetate) revealed the appearance of three new spots (R_{f} 0.6), (R_{f} 0.5) and (R_{f} 0.4) then 100 mL of water was added and the solution extracted with ethyl acetate (3×100 mL). The organic layer was dried with MgSO₄, filtered and evaporated to give 7.46 g of a yellow oil. This oil was purified by silica column chromatography (pure chloroform then 9:1 chloroform/ethyl acetate then 5:5 chloroform/ethyl acetate) to give 1.05 g of pure 84 and 5.38 g of a mixture of silyl trehalose acetates. This mixture was purified by a second by silica column chromatography (95:5 chloroform/ethyl acetate then 9:1 chloroform/ethyl acetate then 5:5 chlorofom/ethyl acetate) to give 84 (280 mg, 15% combined yield) followed by 83 (2.63 g, 35% yield) and 85 (2.0 g, 29% yield)

[α]_{D}²⁵+158 (c=1.0 in CHCl₃); ¹H NMR (400 MHz CHLOROFORM-d) δ ppm 0.01 (3H, s, 1×CH₃), 0.03 (3H, s, 1×CH₃), 0.86 (9H, s, 1×C(CH₃)₃), 2.03 (3H, s, 1×OCOCH₃), 2.03 (3H, s, 1×OCOCH₃), 2.04 (3H, s, 1×OCOCH₃), 2.05 (3H, s, 1×OCOCH₃), 2.08 (3H, s, 1×OCOCH₃), 2.09 (3H, s, 1×OCOCH₃), 2.09 (3H, s, 1×OCOCH₃), 3.63 (2H, m, H-6a and H-6b CH₂OTBS), 3.93 (1H, ddd, J_{5,4}=10.0 Hz, J_{5,6a} 4.4 Hz, J_{5,6b}=3.2 Hz, H-5), 4.00 (1H, dd, J_{6b′,6a′}=12.0 Hz, J_{6b′,5′}=2.0 Hz, H-6b′ OCOCH₃), 4.07 (1H, ddd, J_{5′,4′}=10.0 Hz, J_{5′,6a′} 5.6 Hz, J_{5′,6b′}=2.0 Hz, H-5′), 4.24 (1H, dd, J_{6a′,6b}=12.0 Hz, J_{6a′,5′}=5.6 Hz, H-6a′ CH₂OAc), 4.99 (1H, dd, J_{2,3}=10.0 Hz, J₂₁=3.6 Hz, H-2), 5.05 (1H, dd, J_{4′,5′}=10.0 Hz, J_{4′,3′}=9.6 Hz, H-4′), 5.05 (1H, dd, J_{4,5}=10.0 Hz, J_{4,3}=9.6 Hz, H-4), 5.09 (1H, dd, J_{2′,3′}=10.0 Hz, J_{2′,1′}=4.0 Hz, H-2′), 5.25 (1H, d, J_{1,2}=3.6 Hz, H-1), 5.28 (1H, d, J_{1′,2′}=4.0 Hz, H-1′), 5.46 (2 H, dd, J_{3,2}=10.0 Hz, J_{3,4}=9.6 Hz, H-3 and H-3′); ¹³C NMR (101 MHz CHLOROFORM-d) δ ppm −5.5, −5.4 (CH₃), 18.3 (3 C, 1×C(CH₃)₃), 20.6, 20.6, 20.6, 20.6, 20.6, 20.7, 20.8 (7 C, 7×OCOCH₃), 25.8 (1×C(CH₃)₃), 61.8 (C-6′, OCOCH₃), 61.9 (C-6 CH₂OTBS), 68.1 (C-4′), 68.5 (C-2′), 68.8 (C-4), 69.5 (C-2), 70.1 (C-5′), 70.2 (C-3′), 70.3 (C-5), 70.6 (C-3), 92.2, 92.3 (2×C-1), 169.5, 169.5, 169.5, 169.6, 169.7, 170.1, 170.6 (7×C═O); IR (thin film): ν=2955.7, 2854.6, 1754.6 (C═O), 1369.2, 1221.8, 1142.5, 1037.4, 837.7, 780.5 cm⁻¹; MS (ESI⁺) m/z 768.3 (M+NH₄), 773.2 (M+Na⁺); HRMS (ESI⁺) calcd. for C₃₂H₅₀O₁₈Si (M+Na⁺): 773.2659. Found: 773.2651.

### 6-tert-Butylditnethylsilyl-2,3,4-tri-O-acetyl-α-D-glucopyranosyl 6-tert-butyldimethylsilyl 2,3,4-tri-O-acetyl-α-D-glucopyranoside (84)

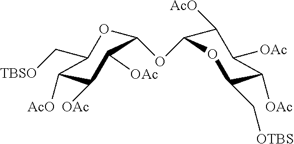

84 was isolated as the upper spot (R_{f} 0.6) TLC (4:1 chloroform/ethyl acetate) of the reaction of D-Trehalose with TBDMS-Cl. (280 mg, 15%).

[α]_{D}²⁵+157 (c=1.0 in CHCl₃); ¹H NMR (400 MHz CHLOROFORM-d) δ ppm 0.02 (6H, s, 2×CH₃), 0.02 (6H, s, 2×CH₃), 0.87 (18H, s, 2×C(CH₃)₃), 2.02 (6H, 2×OCOCH₃), 2.04 (6H, 2×OCOCH₃), 2.08 (6H, 2×OCOCH₃), 3.63 (4H, m, H-6a and H-6b CH₂OTBS), 3.93 (2H, ddd, J_{5,4}=10.0 Hz, J_{5,6a} 4.4 Hz, J_{5,6b}=3.2 Hz, H-5), 5.03 (2H, dd, J_{2,3}=10.4 Hz, J₂,j=4.0 Hz, H-2), 5.07 (2H, dd, J_{4,5}=10.0 Hz, J_{4,3}=9.6 Hz, H-4), 5.25 (2H, d, J_{1,2}=4.0 Hz, H-1), 5.47 (2H, dd, J_{3,2}=10.4 Hz, J_{3,4}=9.6 Hz, H-3); ¹³C NMR (101 MHz CHLOROFORM-d) δ ppm −5.5 (2C, 2×CH₃), −5.5 (2C, 2×CH₃), 18.3 (2C, 2×C(CH₃)₃), 20.7, 20.7, 20.8 (6 C, 6×OCOCH₃), 25.9 (2C, 2×C(CH₃)₃), 61.9 (2C, C-6, C-6′), 68.8 (2C, C-4 C-4′), 69.8 (2C, C-2, C-2′), 70.5 (2C, C-5,C-5′), 70.6 (2C, C-3, C-3′), 92.2 (2C, C-1, C-1′), 169.5, 169.6, 170.2 (6×C═O); IR (thin film): ν=2955.0, 2857.9, 1755.83 (C═O), 1368.7, 1221.4, 1144.5, 1036.0, 837.1, 780.1, cm⁻¹; MS (ESI⁺) m/z 840.3 (M+NH₄⁺), 846.3 (M+Na⁺); HRMS (ESI) calcd. for C₃₆H₆₂O₁₇Si₂ (M+Na⁺): 845.3418. Found: 845.3401.

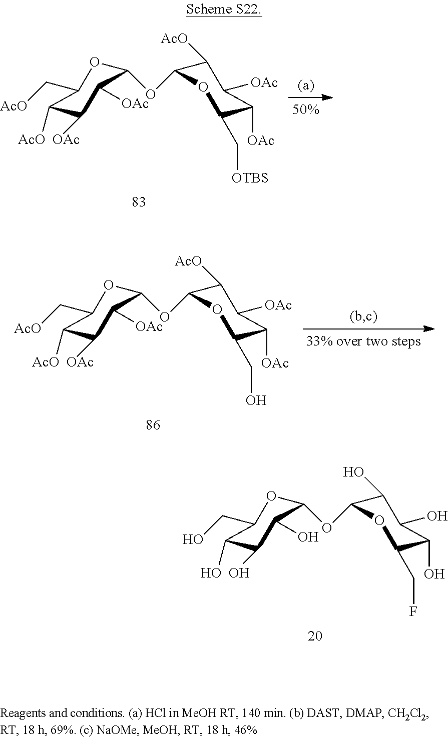

### 2,3,4-tri-O-acetyl-α-D-glucopyranosyl 2,3,4,6-tetra-O-acetyl-α-D-glucopyranoside

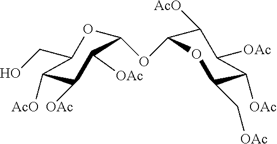

In a 200 mL round-bottomed flask was placed 86 (1.54 g, 2.06 mmol, 1 eq), dry methanol (62 mL) and dry dichloromethane (20 mL), and the solution was cooled to 0° C. Acetyl chloride (293 μL, 4.12 mmol, 2 eq) was added and conversion was monitored by TLC every 10 minutes for 140 minutes. Upon completion, TLC (1:1 ethyl acetate/chloroform) showed disappearance of starting material (R_{f} 0.7) and appearance of a single product (R_{f} 0.5). Satd. NaHCO₃ solution (2 mL) was added, and the mixture was evaporated to dryness to give 1.33 g of oil. This oil was purified by silica column chromatography (99:1 chloroform/ethanol to 98:2 to 97:3 to 95:5) to the titled compound as a white solid (654 mg, 50%).

[α]_{D}²⁵+164 (c, 1.0 in CHCl₃); [Lit. [α]_{D}²¹+167.5 (c=0.7 in CHCl₃)]²⁹; ¹H NMR (400 MHz CHLOROFORM-d) δ ppm 2.03 (3H, 1×OCOCH₃), 2.03 (3H, 1×OCOCH₃), 2.04 (3H, 1×OCOCH₃), 2.05 (3H, 1×OCOCH₃), 2.08 (3H, 1×OCOCH₃), 2.09 (3H, 1×OCOCH₃), 2.09 (3H, 1×OCOCH₃), 3.60 (2H, m, H-6a and H-6b CH₂OH), 3.91 (1H, ddd, J_{5,4}=10.0 Hz, J_{5,6a}=4.6 Hz, J_{5,6b}=2.6 Hz, H-5), 3.99 (1H, dd, J_{6b′,6a′}=12.0 Hz, J_{6b′,5′} 2.0 Hz, H-6b′ CH₂OAc), 4.10 (1H, ddd, J_{5′,4′}=10.4 Hz, J_{5′,6a′}=5.6 Hz, J_{5′,6b′}=2.0 Hz, H-5′), 4.27 (1H, dd, J_{6a′,6b′}=12.0 Hz, J_{6a′,5′}=5.6 Hz, H-6a′ CH₂OAc), 5.01 (1H, dd, J_{2,3}=10.0 Hz, J₂, j=3.6 Hz, H-2), 5.02 (1 H, dd, J_{2′,3′}=10.0 Hz, J_{1′,2′}=4.0 Hz, H-2′), 5.02 (1H, dd, J_{4,5}=10.0 Hz, J_{4,3}=9.6 Hz, H-4), 5.06 (1H, dd, J_{4′,5′}=10.4 Hz, J_{4′,3′}=9.6 Hz, H-4′), 5.29 (1H, d, J_{1,2}=3.6 Hz, H-1), 5.30 (1H, d, J_{1′,2′}=4.0 Hz, H-1′), 5.50 (1H, dd, J_{3′,2′}=10.0 Hz, J_{3′,4′} 9.6 Hz, H-3′), 5.53 (1H, dd, J_{3,2}=10.0 Hz, J_{3,4} 9.6 Hz, H-3); ¹³C NMR (101 MHz CHLOROFORM-d) δ ppm 20.6, 20.6, 20.6, 20.6, 20.7, 20.7, 20.7 (7×OCOCH₃), 60.9 (C-6 OH), 61.7 (C-6′ OCOCH₃), 68.1 (C-4′), 68.4 (C-2′), 68.8 (C-4), 69.7 (C-5′), 69.9 (2C, C-2 and C-3′), 70.0 (C-3), 70.4 (C-5), 92.9 (C-1′), 93.0 (C-1), 169.6, 169.6, 169.9, 169.9, 170.0, 170.4, 170.6 (7×C═O acetates); IR (thin film): ν=3524.5, 3026.3, 2960.7, 1751.2, (C═O) 1370.0, 1221.7, 1164.2, 1036.8, 900.9, 756.8 cm⁻¹; MS (ESI⁺) m/z 653.2 (M+NH₄⁺), 659.2 (M+Na⁺), 695.2 (M+CH₃CN+NH₄⁺); HRMS (ESI⁺) calcd. for C₂₆H₃₆O₁₈ (M+Na⁺): 659.1794. Found: 659.1814.

### 2,3,4-tri-O-acetyl-6-deoxy-6-fluoro-α-D-glucopyranosyl 2,3,4,6-tetra-O-acetyl-α-D-glucopyranoside (87)³⁰

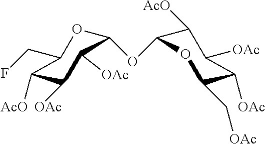

In a 10 mL tube, 86 (111 mg, 0.174 mmol, 1 eq) was dissolved in dry dichloromethane (5 mL). DMAP (44.7 mg, 0.366 mmol, 2.1 eq) and DAST (46 μL, 0.348 mmol, 2.0 eq) were added and the solution was stirred overnight upon which time TLC (1:1 dichloromethane/ethylacetate) revealed complete conversion to product (R_{f} 0.7) and disappearance of starting material (R_{f} 0.5). The mixture was evaporated to dryness and the obtained oil was purified by silica column chromatography (pure chloroform then 95:5 chloroform/ethanol) to give 76.2 mg of the desired compound as a colorless oil (76 mg, 69%).

[α]_{D}²⁵+164 (c=1.0 in CHCl₃) [Lit. [α]_{D}²¹+166 (c=0.65 in CHCl₃)]³⁰; ¹H NMR (400 MHz CHLOROFORM-d) δ ppm 2.04 (3H, 1×OCOCH₃), 2.05 (3H, 1×OCOCH₃), 2.06 (3H, 1×OCOCH₃), 2.08 (3H, 1×OCOCH₃), 2.09 (3H, 1×OCOCH₃), 2.09 (3H, 1×OCOCH₃), 2.09 (3H, 1×OCOCH₃), 3.99 (1H, dd, J_{6b′,6a′}=12.0 Hz, J_{6b′,5′}=2.0 Hz, H-6b′ OCOCH₃), 4.10 (1H, ddd, J_{5′,4}r=10.3 Hz, J_{5′,6a′}=5.6 Hz, J_{5′,6b′}=2.0 Hz, H-5′), 4.14 (1H, dddd, J_{5,F}=21.5 Hz, J_{5,4}=10.3, J_{5,6a}=5.0 Hz, J_{5,6b}=2.8 Hz, H-5), 4.27 (1H, dd, J_{6a′,6b′}=12.0 Hz, J_{6a′,5′}=5.6 Hz, H-6a′OCOCH₃), 4.39 (1H, ddd, J_{6b,F}=47.1 Hz, J_{6b,6a}=10.4 Hz, J_{6b,5}=2.8 Hz, H-6b CH₂F), 4.42 (1H, ddd, J_{6,F}=47.1 Hz, J_{6a,6b}=10.4 Hz, J_{5,6a}=5.0 Hz, H-6a CH₂F), 5.02 (1H, dd, J_{4,5}=10.3 Hz, J_{4,3}=9.5 Hz, H-4), 5.02 (1H, dd, J_{2,3′}=10.3 Hz, J_{2′,1′}=3.9 Hz, H-2′), 5.06 (1H, dd, J_{2,3}=10.3 Hz, J₂,j=3.9 Hz, H-2), 5.07 (1H, dd, 10.3 Hz, J_{4′,3′}=9.5 Hz, H-4′), 5.28 (1H, d, J_{1,2}=3.9 Hz, H-1), 5.29 (1H, d, J_{1′,2′}=3.9 Hz, H-1′), 5.48 (1H, dd, J_{3,2}=10.3 Hz, J_{3,4}=9.5 Hz, H-3), 5.50 (1H, dd, J_{4′,5′}=10.3 Hz, J_{3′,4′}=9.5 Hz, H-3′); ¹³C NMR (125 MHz CHLOROFORM-d) δ ppm 20.5, 20.6, 20.6, 20.6, 20.6, 20.7, 20.7 (7×OCOCH₃), 61.7 (C-6′ OCOCH₃), 68.4 (C-2′), 68.7 (1C, d, J_{C-4,F}=7.0 Hz C-4), 69.0 (1C, d, J_{C-5,F}=19 Hz C-5), 69.7 (C-5′), 69.8 (2C, C-3 and C-3′), 70.0 (C-2), 81.3 (1C, d, J_{C-6,F}=175 Hz, C-6), 92.9 (C-1′), 93.1 (C-1), 169.5, 169.5, 169.6, 169.7, 169.9, 170.0, 170.6 (7×C═O); ¹⁹F NMR (376.5 MHz CHLOROFORM-d) δ ppm: −230.6, dt, J_{6,F}=47.1 Hz, J_{5,F}=21.5 Hz; IR (thin film): ν=2959, 1752, 1643, 1558, 1538, 1435, 1370, 1219, 1039, 803 cm⁻¹; MS (ESI) m/z 661.2 (M+Na⁺); HRMS (ESI⁺) calcd. for C₂₆H₃₅O₁₇F (M+Na⁺): 661.1750. Found: 661.1742.

### 6-deoxy-6-fluoro-α-D-glucopyranosyl-α-D-glucopyranoside (20)³¹

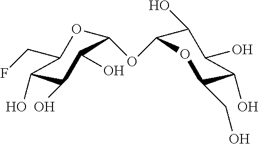

In a 25 mL flask, 87 (44.5 mg, 0.069 mmol, 1 eq) was dissolved in dry methanol (10 mL). Dry sodium methoxide (21.6 mg, 0.4 mmol, 6 eq) was added and the solution stirred overnight upon which time TLC (1:4:4 water:isopropanol:ethyl acetate) showed complete conversion to a single product (R_{f} 0.4) and disappearance of starting material (R_{f} 1). Reaction was neutralized with DOWEX, 50WX8 (H⁺ form) cation exchange resin and then was filtered and evaporated to dryness to give 20.0 mg of a solid. This solid was purified by silica column chromatography (pure EtOAc then 1:4:4 water:isopropanol:ethyl acetate) to give a yellow compound that was discolored with activated charcoal, filtered and evaporated to afford the desired compound as a white, amorphous solid (11 mg, 46%) yield.

[α]_{D}²⁵+124 (c=0.2 in H₂O) [Lit. [α]_{D}²¹+174.2 (c=1.0 in MeOH)]³¹¹H NMR (400 MHz DEUTERIUM OXIDE) δ ppm: 3.32 (1H, dd, J_{4′,5′}=10.4 Hz, J_{4′,3′}=9.2 Hz, H-4′), 3.43 (1H, dd, J_{4,5}=10.0 Hz, J_{4,3}=9.6 Hz, H-4), 3.55 (1H, dd, J_{2′,3′}=9.8 Hz, J₂′,1′=4.0 Hz, H-2′), 3.58 (1H, dd, J_{2,3}=9.8 Hz, J₂,j=4.0 Hz, H-2), 3.67 (1 H, dd, J_{6b′,6a′}=12.0 Hz, J_{6b′,5′}=5.0 Hz, H-6b′), 3.73 (1H, m, H-5′), 3.75 (1H, dd, J_{3′,2′}=9.8 Hz, J_{3′,4′}=9.2 Hz, H-3′), 3.76 (1H, dd, J_{6a′,6b′}=12.0 Hz, J_{6a′,5′}=2.6 Hz, H-6a′), 3.78 (1H, dd, J_{3,2}=9.8 Hz, J_{3,4}=9.6 Hz, H-3′), 3.88 (1H, dddd, J_{5,F}=28.7 Hz, J₅,4=10.0 Hz, J_{5,6a}=3.0 Hz, J_{5,6b}=1.8 Hz, H-5), 4.59 (1H, ddd, J_{6b,F}=47.6 Hz, J_{6b,6a}=10.8 Hz, J_{6b,5}=1.8 Hz, H-6b), 4.65 (1H, ddd, J_{6a,F}=47.6 Hz, J_{6a,6b}=10.8 Hz, J_{6a,5}=3.0 Hz, H-6a), 5.05 (1H, d, J_{1′,2′}=4.0 Hz, H-1′), 5.09 (1H, d, J_{1,2}=4.0 Hz, H-1), ¹³C NMR (101 MHz DEUTERIUM OXIDE) δ ppm 60.8 (C-6′), 68.1 (C-4′), 68.9 (1, C, d, J_{C-4,F}=6.2 Hz, C-4), 70.0 (C-4′), 71.2, 71.2 (C-2 and C-2′), 71.4 (1C, d, J_{C-5,F}=12.0 Hz, C-5), 72.6 (C-5′), 72.7 (C-3), 72.8 (C-3′), 82.4 (1C, d, J_{C-6,F}=168 Hz, C-6-F), 93.8 (C-1 or C-1′), 93.9 (C-1 or C-1′); ¹⁹F NMR (377 MHz DEUTERIUM OXIDE) δ ppm: −235.6 (dt, J_{6,F}=47.6 Hz, J_{5,F} 28.7 Hz); IR (KBr): ν=3421 (OH), 2925, 1683, 1600, 1472, 1261, 1150, 1106, 1077, 1030, 990 cm⁻¹; MS (ESI⁺) m/z 367.1 (M+Na⁺), MS (ESI−) m/z 343.1 (M−H)⁻; HRMS (ESI⁺) calcd. for C₁₂H₂₀O₁₀F (M−H)⁻: 343.1046. Found: 343.1045

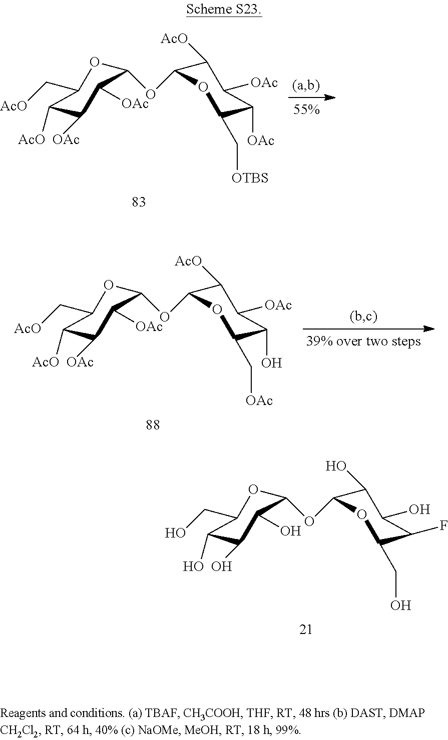

### 2,3,6-tri-O-acetyl-α-D-glucopyranosyl 2,3,4,6-tetra-O-acetyl-α-D-glucopyranoside (88)

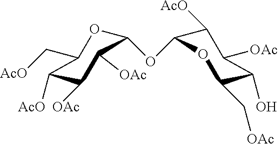

In a 100 mL flask was dissolved 83 (1.5 g, 2.01 mmol, 1 eq) in dry THF (20 mL). Glacial acetic acid (230 μL, 4.0 mmol, 2 eq) was added followed a 1M TBAF solution in THF (4.0 mL, 4.03 mmol, 2 eq). After 24 h TLC (1:1 dichloromethane/ethyl acetate) showed some starting material (R_{f} 0.8) remaining and reaction is completed after 48 hours (R_{f} 0.5). The mixture was evaporated and dried under high vacuum to give 4.0 g of oil. This oil was purified by silica column chromatography (98:2 chloroform/ethanol) to give the titled compound as a amorphous white solid (1.1 g, 85%).

[α]_{D}²⁵+150 (c=1.0 in CHCl₃); ¹H NMR (400 MHz CHLOROFORM-d) δ ppm 2.03 (3H, 1×OCOCH₃), 2.05 (3H, 1×OCOCH₃), 2.08 (3H, 1×OCOCH₃), 2.09 (3 H, 1×OCOCH₃), 2.10 (3H, 1×OCOCH₃), 2.11 (3H, 1×OCOCH₃), 2.14 (3H, 1×OCOCH₃), 3.56 (1H, dd, J_{4,5}=10.0 Hz, J_{4,3}=9.2 Hz, H-40H), 3.91 (1H, ddd, J_{5,4}=10.0 Hz, J_{5,6a} 5.6 Hz, J_{5,6b}=2.0 Hz, H-5), 4.01 (1H, dd, J_{6b′,6a′=}12.0 Hz, J_{6b′,5′}=2.0 Hz, H-6b′), 4.06 (1H, ddd, J_{5′4′}=10.0 Hz, J_{5′,6a′}=5.6 Hz, J_{5′,6b′}=2.0 Hz, H-5′), 4.23 (1H, dd, J_{6b,6a}=12.0 Hz, J_{6b,5}=2.0 Hz, H-6b), 4.24 (1H, dd, J_{6a′,6b′}=12.0 Hz, J_{6a,5′}=5.6 Hz, H-6a′), 4.41 (1H, dd, J_{6a,6b}=12.0 Hz, J_{6a,5}=5.6 Hz, H-6a) 5.00 (1H, dd, J_{3,2}=10.4 Hz, J_{2,1}=3.6 Hz, H-2), 5.02 (1H, dd, J_{3′,2′}=10.4 Hz, J_{2′,1′}=4.0 Hz, H-2′), 5.04 (1H, dd, J_{4′,5′}=10.0 Hz, J_{4′,3′}=9.2 Hz, H-4′), 5.24 (1H, d, J_{1,2}=3.6 Hz, H-1), 5.30 (1H, d, J_{1′,2′}=4.0 Hz, H-1′), 5.31 (1H, dd, J_{3,2}=10.4 Hz, J_{3,4}=9.2 Hz, H-3), 5.49 (1H, dd, J_{3′,2′}=10.4 Hz, J_{3′4′}=9.2 Hz, H-3′); ¹³C NMR (101 MHz CHLOROFORM-d) δ ppm 20.6, 20.6, 20.6, 20.6, 20.7, 20.7, 20.9 (7×OCOCH₃), 61.7 (C-6′), 62.6 (C-6), 68.1 (C-4′), 68.5 (C-2′), 69.5 (2C, C-2 and C-5′), 69.8 (C-5), 69.9 (C-3′), 70.8 (C-3), 73.1 (C-4 OH), 91.9 (C-1), 92.2 (C-1′), 169.6, 169.6, 169.7, 170.0, 170.6, 171.3, 172.1 (7×C═O acetates); IR (thin film): ν=3568, 3305, 3025, 2959, 1748, (C═O), 1370, 1223, 1161, 1039, 902, 757 cm⁻¹; MS (ESI⁺) m/z 695.2 (M+CH₃CN+NH₄); HRMS (ESI⁺) calcd. for C₂₆H₃₆O₁₈ (M+Na⁺): 659.1794. Found: 659.1788.

### 2,3,6-tri-O-acetyl-4-deoxy-4-fluoro-α-D-galactopyranosyl 2,3,4,6-tetra-O-acetyl-α-D-glucopyranoside (89)³²

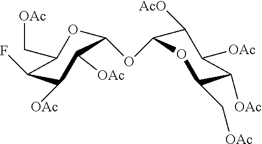

In a 10 mL tube, 88 (87.2 mg, 0.137 mmol, 1 eq) and DMAP (35.1 mg, 0.288 mmol, 2.1 eq) were dissolved in anhydrous dichloromethane (3 mL). The solution was cooled to −20° C. and DAST (36 μL, 0.274 mmol, 2 eq) was added. After 64 h, TLC (1:1 CH₂Cl₂:EtOAc) showed conversion to product (R_{f}=0.72). The mixture was concentrated under reduced pressure and purified by silica column chromatography (98:2 CHCl₃:EtOH followed by 95:5 and 90:10) to give the desired compound (34.5 mg, 40%) as a clear oil.

[α]_{D}²⁵+138 (c=0.5 in CHCl₃) [Lit. [α]_{D}²¹+168 (c=1.0 in CHCl₃)]³²; ¹H NMR (400 MHz CHLOROFORM-d) δ ppm 2.04, 2.05, 2.06, 2.08, 2.09, 2.09, 2.09 (7×3H, s, OCOCH₃), 4.01 (1H, dd, J_{6b′,6a′}=12.0 Hz, J_{6b′,′}=2.0 Hz, H-6b′), 4.05 (1H, ddd, J_{5′,4′}=10.0 Hz, J₅=5.6 Hz, J₅=2.0 Hz, H-5′), 4.15 (2H, m, H-5, H-6b), 4.24 (1H, dd, J_{6a′,6b,=}12.0 Hz, J_{6a′,5′}=5.6 Hz, H-6a′), 4.30 (1H, m, H-6a), 5.02 (1H, dd, J_{4,F}=50.2 Hz, J_{4,3}=2.4 Hz, H-4 F), 5.03 (1H, dd, J_{2′,3′}=10.0 Hz, J_{1′,2′}=4.0 Hz, H-2′), 5.04 (1H, app t, J_{4′,5′}=J_{4′,3′}=10.0 Hz, H-4′), 5.25 (1H, ddd, J_{3,F}=25.7 Hz J_{3,2}=10.0 Hz, J_{3,4}=2.4 Hz, H-3), 5.29 (1H, d,=4.0 Hz, H-1′), 5.35 (3H, d, J_{1,2}=3.8 Hz, H-1), 5.37 (1H, dd, J_{2,3}=10.0 Hz, J_{2,1}=3.8 Hz, H-2), 5.47 (1H, dd, J_{3′,2′}=10.0 Hz, J_{3′,4′}=10.0 Hz, H-3′); ¹³C NMR (125 MHz CHLOROFORM-d) δ ppm 20.5, 20.6, 20.6, 20.6, 20.6, 20.6, 20.8, (7×CH₃ acetates), 61.6 (1C, d, J_{C-6,F}=6.3 Hz C-6), 61.7 (C-6′), 66.7 (C-2), 67.5 (1C, d, J_{C-5,F}=18 Hz, C-5), 68.2 (C-4′), 68.3 (1C, d,=18 Hz, C-3), 68.5 (C-2′), 69.7 (C-5′), 69.8 (C-3′), 86.4 (1C, d, J_{C-4,F}=185 Hz, C-4, CH—F), 92.5 (C-1′), 92.9 (C-1), 169.5, 169.7, 170.0, 170.3, 170.3, 170.3, 170.6 (7×C═O acetates); ¹⁹F NMR (1H) (377 MHz CHLOROFORM-d) δ ppm: −219.2, ddd, J_{F-H4}=50.8 Hz, J_{F-H5}=28.9 Hz, J_{F-H3}=25.7 Hz; IR (thin film): v=2961.9, 1749.9, 1645.0, 1538.9, 1434.6, 1371.5, 1224.7, 1038.1, 733.6 cm⁻¹; MS (ESI⁺) m/z 697.2 (M+CH₃CN+NH₄⁺); HRMS (ESI) calcd. for C₂₆H₃₅O₁₇F (M+Na⁺): 661.1750. Found: 661.1765.

### 4-deoxy-4-fluoro-α-D-galactopyranosyl α-D-glucopyranoside (21)³²

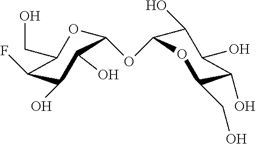

89 (16.2 mg, 0.047 mmol, 1 eq) was dissolved in dry methanol (5 mL) and dry sodium methoxide (10.8 mg, 0.28 mmol, 6 eq) was added. After stirring overnight, TLC (4:4:1 ethyl acetate:isopropanol:water) showed the formation of a single compound (R_{f} 0.28). Reaction was neturalized with DOWEX 50WX8 (H⁺ form) cation exchange resin and the solution was filtered and evaporated to give 4-deoxy-4-fluoro-α-D-galactopyranosyl α-D-glucopyranoside as a clear oil (10.3 mg, 100%).

[α]_{D}²⁵+176 (c=0.1 in H₂O) [Lit. [α]_{D}²¹+172.6 (C=0.5 in CH₃OH)]³²; ¹H NMR (400 MHz DEUTERIUM OXIDE) δ ppm 3.33 (1H, dd, J_{4′,5′}=10.0 Hz, J_{4′,3′}=9.2 Hz, H-4′), 3.53 (1H, dd, J_{2′,3′}=10.0 Hz, J_{1′,2′}=4.0 Hz, H-2′), 3.67 (1H, dd, J_{6b′,6a′}=12.0 Hz, J_{6b′,5′}=5.6 Hz, H-6b′), 3.72 (2H, m, H-6a and H-6b), 3.75 (1H, dd, J_{3′,2′}=10.0 Hz, J_{3′,4′}=9.2 Hz, H-3′), 3.75 (1H, m, H-5′), 3.76 (1H, dd, J_{6a′,6b′}=12.0 Hz, J_{6a′,5′}=2.2 Hz, H-6a′), 3.85 (1H, dd, J_{2,3}=10.4 Hz, J_{1,2}=4.0 Hz, H-2), 4.00 (1H, ddd, J_{3,F}=30.0 Hz, J_{3,2}=10.4 Hz J_{3,4}=2.8 Hz, H-3), 4.04 (1H, dt, J_{5,F}=32.2 Hz, J_{5,6a}=J_{5,6b}=6.4 Hz, H-5), 4.80 (1H, dd, J_{4,F}=50.5 Hz, J_{4,3}=2.8 Hz, H-4), 5.08 (1H, d, J_{1′,2′}=4.0 Hz, H-1′), 5.15 (1H, d, J_{1,2}=4.0 Hz, H-1); ¹³C NMR (101 MHz DEUTERIUM OXIDE) δ ppm 60.4 (d, J_{C-6,F}=5.8 Hz C-6), 60.8 (C-6), 68.1 (d, J_{C-3F}=17 Hz C-3), 68.2 (C-2), 70.0 (C-4′), 70.5 (d, J_{C-5F}=18 Hz C-5), 71.3 (C-2′), 72.5 (C-5′), 72.8 (C-3′), 90.7 (d, J_{C-F}=177 Hz, C-4-F), 93.8 (2×C-1); ¹⁹F NMR (377 MHz DEUTERIUM OXIDE) δ ppm: −219.7, ddd, J_{F-H4}=50.5 Hz, J_{F-H5}=32.2 Hz, J_{F-H3}=30.0 Hz; IR (KBr): ν=3430 (OH), 2928, 1635.98, 1419, 1350, 1260, 1151, 1102, 1077, 1050, 1013 cm⁻¹; MS (ESI⁺) m/z 367.1 (M+Na⁺), MS (ESI−) m/z 343.1 (M−H)⁻; HRMS (ESI⁺) calcd. for C₁₂H₂₁O₁₀F (M+Na⁺): 367.1011 Found: 367.1008.

### 6-deoxy-6-bromo-α-D-glucopyranosyl-α-D-glucopyranoside (19) ^{33,34}

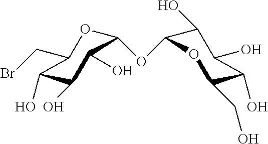

The compound was synthesized according to the literature³³. In a 50 mL flask, anhydrous d-trehalose (1.52 g, 4.43 mmol, 1 eq) was dissolved in dry DMF (15 mL). Triphenylphosphine (2.32 g, 8.87 mmol, 2 eq) was added followed by NBS (1.57 g, 8.87 mmol, 2 eq). The mixture was stirred overnight at room temperature and 24 h at +60° C. and evaporated to give 5.00 g of a yellow oil containing a mixture of expected compound along with unreacted trehalose and corresponding dibromo. This oil was purified by column chromatography using (EtOAc: MeOH 100:0, 90:10 and 80:20) to give (408.3 mg, 20%) and the titled compound as a brownish, amorphous solid (594.0 mg, 33%).

[α]_{D}²¹+134 (c=1 in (H₂O) [Lit. [α]_{D}²¹+180.8 (C=0.7 in CH₃OH)]³⁴; ¹H NMR (400 MHz DEUTERIUM OXIDE) δ ppm: 3.33 (1H, app t, J_{4′,5′}=J_{4′,3′}=9.8 Hz, H-4′), 3.38 (1H, app t, J_{4,5}=J_{4,3}=9.8 Hz, H-4), 3.53 (1H, dd, J_{2,3}=9.8 Hz, J_{2,1}=3.8 Hz, H-2), 3.56 (1H, dd, J_{2′,3′}=9.8 Hz, J_{2′,1′}=3.8 Hz, H-2′), 3.56 (1H, dd, J_{6b,6a}=11.4 Hz, J_{6b,5}=5.4 Hz, H-6b CH₂Br), 3.64 (1H, dd, J_{6b′,6a′=}11.6 Hz, J_{6b′,5′}=5.0 Hz, H-6b′ CH₂OH), 3.68 (1H, dd, J_{6a,6b}=11.4 Hz, J_{6a,5}=2.8 Hz, H-6a CH₂Br), 3.70 (1H, app t, J_{3′,2′}=J_{3′,4′}=9.8 Hz, H-3′), 3.73 (1H, dd, J_{6a′,6b′}=11.4 Hz, J_{6a′,5′}=2.6 Hz, H-6a′ CH₂OH), 3.73 (1H, m, H-5′), 3.75 (1H, app t, J_{3,2}=J_{3,4}=9.8 Hz, H-3), 3.87 (1H, ddd, J_{5,4}=9.8 Hz, J_{5,6b}=5.4 Hz, J_{5,6a}=2.8 Hz, H-5), 5.08 (1H, d, J_{1′,2′}=3.8 Hz, H-1′), 5.10 (1H, d, J_{1,2}=3.8 Hz, H-1); ¹³C NMR (126 MHz DEUTERIUM OXIDE) δ ppm 34.1 (C-6 CH₂Br), 60.8 (C-6′ CH₂OH), 69.9 (C-4′), 71.0 (C-4), 71.2, 71.2 (C-2 and C-2′), 71.9 (C-5), 72.4 (C-3), 72.5 (C-5′), 72.8 (C-3′), 93.6 (C-1), 93.8 (C-1′); IR (KBr): ν=3421, 2925, 1653, 1636, 1419, 1261, 1149, 1103, 992, 939 cm⁻¹; MS (ESI−) m/z 403.0 and 405.0 (M−H)⁻; HRMS (ESI⁻) calcd. for C₁₂H₂₀O₁₀⁷⁹Br⁻ (M−H)⁻: 403.0245. Found: 403.0241, calcd. for C₁₂H₂₀O₁₀⁸¹Br⁻ (M−H)⁻: 405.0227. Found: 405.0219.

### REFERENCES

¹ Rohde, K., Yates, R. M., Purdy, G. E., and Russell, D. G., Mycobacterium tuberculosis and the environment within the phagosome. Immunol Rev 219, 37 (2007).
² Boucau, Julie et al., A coupled assay measuring Mycobacterium tuberculosis antigen 85C enzymatic activity. Analytical Biochemistry 385 (1), 120 (2009); Sanki, A. K., Boucau, J., Ronning, D. R., and Sucheck, S. J., Antigen 85C-mediated acyl-transfer between synthetic acyl donors and fragments of the arabinan. Glycoconj J (2008).
³ Belisle, J. T. et al., Role of the major antigen of Mycobacterium tuberculosis in cell wall biogenesis. Science 276 (5317), 1420 (1997).
⁴ Geoghegan, Kieran F. et al., Spontaneous [alpha]-N-6-Phosphogluconoylation of a “His Tag” in Escherichia coli:The Cause of Extra Mass of 258 or 178 Da in Fusion Proteins. Analytical Biochemistry 267 (1), 169 (1999).
⁵ Nguyen, Liem, Chinnapapagari, Satheesh, and Thompson, Charles J., FbpA-Dependent Biosynthesis of Trehalose Dimycolate Is Required for the Intrinsic Multidrug Resistance, Cell Wall Structure, and Colonial Morphology of Mycobacterium smegmatis. J. Bacteriol. 187 (19), 6603 (2005).
⁶ Khan, R. et al., Regioselective Deacetylation of Fully Acetylated Mono- and Di-Saccharides With Hydrazine Hydrate. Australian Journal of Chemistry 49 (3), 293 (1996).
⁷ Saito, H., Yoshikawa, H., and Nishimura, Y., Studies on lignan lactone antitumor agents. I. Synthesis of aminoglycosidic lignan variants related to podophyllotoxin. Chemical and Pharmaceutical Bulletin 34 (9), 3733 (1986).
⁸ Withers, S. G., MacLennan, D. J., and Street, I. P., The synthesis and hydrolysis of a series of deoxyfluoro-d-glucopyranosyl phosphates. Carbohydrate Research 154 (1), 127 (1986).
⁹ Shelling, Judith G. et al., 2′-fluoromaltose: Synthesis and properties of 4-O-(2-deoxy-2-fluoro-[alpha]—glucopyranosyl)—glucopyranose, and the crystal structure of 2,3-di-O-acetyl-1,6-anhydro-4-O-(3,4-tri-O-acetyl-2-deoxy-2-fluoro-[alpha]—glucopyranosyl)-[beta]—glucopyranose. Carbohydrate Research 132 (2), 241 (1984); Szarek, Walter A., Hay, George W., Doboszewski, Bogdan, and Perlmutter, Milton M., Reaction of 1,2-anhydro-3,4:5,6-di-O-isopropylidene-1-C-nitro—mannitol with potassium hydrogenfluoride in ethylene glycol: a synthesis of 2-deoxy-2-fluoro—glucose. Carbohydrate Research 155, 107 (1986).
¹⁰ Glanzer, B. I. and Csuk, R., Reaction of pyranoid and furanoid aldonolactones with chloromethyltrimethylsilane-derived reagents. Carbohydrate Research 220, 79 (1991).
¹¹ RajanBabu, T. V. and Reddy, G. S., 1-Methylene sugars as C-glycoside precursors. The Journal of Organic Chemistry 51 (26), 5458 (1986).
¹² Griffin, Frank K., Paterson, Duncan E, Murphy, Paul V, and Taylor, Richard J K, (2002), Vol. 2002, pp. 1305.
¹³ Li, Xiaoliu, Ohtake, Hiro, Takahashi, Hideyo, and Ikegami, Shiro, A facile synthesis of 1′-C-alkyl-[alpha]-disaccharides from 1-C-alkyl-hexopyranoses and methyl 1-C-methyl-hexopyranosides. Tetrahedron 57 (20), 4297 (2001).
¹⁴ Zhu, X., Jin, Y., and Wickham, J., (2007), Vol. 72, pp. 2670.
¹⁵ Kuzuhara, Hiroyoshi and Fletcher, Hewitt G., Syntheses with partially benzylated sugars. VIII. Substitution at carbon-5 in aldose. The synthesis of 5-O-methyl-D-glucofuranose derivatives. J. Org. Chem. 32 (8), 2531 (1967).
¹⁶ Dondoni, Alessandro and Scherrmann, Marie-Christine, Thiazole-Based Synthesis of Formyl C-Glycosides. J. Org. Chem. 59 (21), 6404 (1994).
¹⁷ Withers, Stephen G., MacLennan, David J., and Street, Ian P., The synthesis and hydrolysis of a series of deoxyfluoro—glucopyranosyl phosphates. Carbohydrate Research 154 (1), 127 (1986); Foster, A. B; Hems, R; Hall, L. D., Fluorinated carbohydrates. Part V. Nuclear magnetic resonance data on pyranose and furanose derivatives of 3-deoxy-3-fluoro-D-glucose. Canadian Journal of Chemistry 24 (48), 8 (1970).
¹⁸ Myszka, Henryk, Bednarczyk, Dorota, Najder, Maria, and Kaca, Wieslaw, Synthesis and induction of apoptosis in B cell chronic leukemia by diosgenyl 2-amino-2-deoxy-[beta]—glucopyranoside hydrochloride and its derivatives. Carbohydrate Research 338 (2), 133 (2003).
¹⁹ Boullanger, Paul et al., The use of N-alkoxycarbonyl derivatives of 2-amino-2-deoxy-1-glucose as donors in glycosylation reactions. Carbohydrate Research 202, 151 (1990).
²⁰ Namme, Rie., Hideyo, Takashi Mitsugi., and Ikegami, Takahashi Shiro., Development of Ketoside-Type Analogues of Trehalose by Using?—Stereoselective O-Glycosidation of Ketose. European Journal of Organic Chemistry 2007 (22), 3758 (2007).
²¹ Baer, Hans H., Breton, Roger L., and Shen, Yaping, Synthesis of a trehalose homolog, 6-deoxy-[alpha]—gluco-heptopyranosyl 6-deoxy-[alpha]—gluco-heptopyranoside, and the corresponding bis(heptosiduronic acid). Carbohydrate Research 200, 377 (1990).
²² Leatherdale, C. A., Woo, W. K., Mikulec, F. V., and Bawendi, M. G., On the Absorption Cross Section of CdSe Nanocrystal Quantum Dots. The Journal of Physical Chemistry B 106 (31), 7619 (2002).
²³ Viebel. Biochem. Z. 239 (350) (1931).
²⁴ Min Wang, Peng-Fei Tu Zhi-Dong Xu Xiao-Lin Yu Ming Yang, Design and Synthesis of Guanidinoglycosides Directed against the TAR RNA of HIV-1. Helvetica Chimica Acta 86 (7), 2637 (2003).
²⁵ Rodriguez, Miguel Angel et al., Synthesis of 2-Iodoglycals, Glycals, and 1,1′-Disaccharides from 2-Deoxy-2-iodopyranoses under Dehydrative Glycosylation Conditions. The Journal of Organic Chemistry 72 (23), 8998 (2007).
²⁶ Lin, Fiona L., van Halbeek, Herman, and Bertozzi, Carolyn R., Synthesis of mono- and dideoxygenated [alpha],[alpha]-trehalose analogs. Carbohydrate Research 342 (14), 2014 (2007).
²⁷ Chenault, H. Keith, Mandes, Robert F., and Hornberger, Keith R., Synthetic Utility of Yeast Hexokinase. Substrate Specificity, Cofactor Regeneration, and Product Isolation. Journal of Organic Chemistry 62, 331 (1997).
²⁸ Gibson, Robert P. et al., Insights into Trehalose Synthesis Provided by the Structure of the Retaining Glucosyltransferase OtsA. Chemistry & Biology 9 (12), 1337 (2002).
²⁹ Szurmai, Zoltán, Kerékgyártó, János, Harangi, János, and Lipták, András, Glycosylated trehalose. Synthesis of the oligosaccharides of the glycolipid-type antigens from Mycobacterium smegmatis. Carbohydrate Research 164, 313 (1987).
³⁰ Defaye, Jacques et al., Asymmetric acetalation of [alpha],[alpha]-trehalose: Synthesis of [alpha]—galactopyranosyl [alpha]—glucopyranoside and 6-deoxy-6-fluoro-[alpha]—glucopyranosyl [alpha]—glucopyranoside. Carbohydrate Research 63, 41 (1978).
³¹ Hadfield, Anthony F., Hough, Leslie, and Richardson, Anthony C., The synthesis of 6-deoxy-6-fluoro-[alpha],[alpha]-trehalose and related analogues. Carbohydrate Research 63, 51 (1978).
³² Hadfield, Anthony F., Hough, Leslie, and Richardson, Anthony C., The syntheses of 4,6-dideoxy-4,6-difluoro- and 4-deoxy-4-fluoro-[alpha],[alpha]-trehalose. Carbohydrate Research 71 (1), 95 (1979).
³³ Wang, Min et al., [alpha],[alpha]-Trehalose derivatives bearing guanidino groups as inhibitors to HIV-1 Tat-TAR RNA interaction in human cells. Bioorganic & Medicinal Chemistry Letters 14 (10), 2585 (2004).
³⁴ Hanessian, S, and Lavelle, P., Synthesis of 6-amino-6-deoxy-α,α-a Positional Isomer of Trehalosamine. The Journal of Antibiotics 25 (11), 683 (1972).

1. A compound for labelling mycobacteria, which compound comprises a label and a substrate, which label can be detected by a detector responsive to the presence of the label, optionally after applying a stimulus, characterised by the compound being able to engage with the active site of Antigen 85B (Ag85B) such that it can form simultaneous hydrogen bonds with two or more amino acids in the active site selected from Arg 43, Trp 264, Ser126, His 262 and Leu 42, or the corresponding amino acids in Antigen 85A (Ag85A) or Antigen 85C (Ag85C), at least one of which is with Ser126.

2. A compound as claimed in claim 1, in which the substrate is a carbohydrate or derivative thereof comprising 1 to 6 monosaccharide units.

3. A compound as claimed in claim 1, in which the carbohydrate is a mono or disaccharide or derivative thereof.

4. A compound as claimed in claim 2, with Formula I: 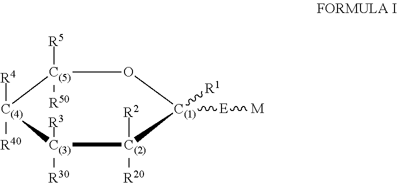
in which;
M is a carbohydrate or derivative thereof comprising 1 to 5 monosaccharide units, linked to C₍₁₎ through bridge group E, in either α or β configurations;
R¹ is selected from H, -L, —X, —CYY′L, —CYY′X;
One of the R² and R²⁰ is —H and the other is selected from —OH, -L, —X, —CYY′L, —CYY′X;
One of R³ and R³⁰ is —H and the other is selected from —OH, -L, —X, —CYY′L, —CYY′X;
One of R⁴ and R⁴⁰ is —H and the other is selected from —OH, -L, —X, —CYY′L, —CYY′X;
One of R⁵, and R⁵⁰ are —H, and the other is selected from CYY′L or —CYY′X;—
X is an optional derivative group, and is selected from halides, R^{a} groups, —Z—H groups, groups of general formula —Z—R^{a}, groups of general formula WH₂₋ₓR^{a}ₓ, groups of general formula —C(Z′)Z″—H, —C(Z)—R^{a}, —C(Z′)Z″—R^{a} and —C(Z)—WH_{1-y}R^{a}_{y}; in which;
R^{a} is, at each occurrence, an optionally substituted linear or branched alkyl, alkenyl and alkynyl groups or an optionally substituted aromatic group; where R^{a} comprises one or more substituents, such substituents are selected from halides, —ZH, Z—R^{b}, WH₂₋ₓR^{b}x, where R^{b} at each occurrence is selected from optionally substituted linear or branched alkyl, alkenyl, alkynyl and aromatic groups;
Z, Z′ and Z″ at each occurrence is a Group 16 element;
W is a Group 15 element;
x is 0, 1 or 2, and y is 0 or 1;
E is selected from one or more of (a) a Group 16 element; (b) a group comprising a Group 15 element with formula WH_{(1-(y+y′))}R^{a}_{y}L_{y}′ in which W is the Group 15 element, y and y′ are independently 0 or 1, and y+y′ is no more than 1; (c) a group comprising a Group 14 element of general formula VX′_{(2-(x+x′)}R^{a}ₓL_{x′} in which V is the Group 14 element, X′ is at each occurrence H, OH or X, x and x′ are individually 0, 1 or 2, x+x′ being no more than 2.
Y and Y′ are independently H or X, with the proviso that the carbon atom to which they are bound has no more than one directly bound O, Z and W atoms;
wherein there is at least one label group L on the substrate molecule and/or at least one carbon atom in the molecule is ¹³C or ¹⁴C enriched, and/or at least one hydrogen atom in the molecule is ²H or ³H enriched.

5. A compound as claimed in claim 4, of Formula II; 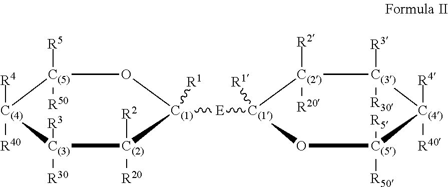
Where;
E is a bridging group as defined in claim 4, and each of the two monosaccharides are either α or β linked.
R¹ and R¹ are independently selected from -L, —X, —CYY′L, —CYY′X;
One of R² and R²⁰ is —H and the other is selected from —OH, -L, —X, —CYY′L, —CYY′X;
One of R²′ and R²⁰′ is —H and the other is selected from —OH, -L, —X, —CYY′L, —CYY′X;
One of R³ and R³⁰ is —H and the other is selected from —OH, -L, —X, —CYY′L, —CYY′X;
One of R³ and R³⁰′ is —H and the other is selected from —OH, -L, —X, —CYY′L, —CYY′X;
One of Wand R⁴⁰ is —H and the other is selected from —OH, -L, —X, —CYY′L, —CYY′X;
One of R⁴′ and R⁴⁰′ is —H and the other is selected from —OH, -L, —X, —CYY′L, —CYY′X;
One of R⁵ and R⁹ is −11, and the other is selected from CYY′L or —CYY′X;—
One of R⁵ and R⁵⁰′ is —H, and the other is selected from CYY′L or —CYY*X;—
at least one of R⁴ and R⁴′ is H and/or at least one of R⁴⁰ and R⁴⁰′ is able to form hydrogen bonds;
X is optional and is as defined in claim 4;
Y and Y′ are independently H or X, with the proviso that the carbon atom to which they are bound has no more than one directly bound O, Z and W atoms; and
wherein there is either at least one label group L on the molecule and/or one or more carbon atoms in the molecule is ¹³C or ¹⁴C enriched and/or at least one hydrogen atom in the molecule is ²H or ³H enriched.

6. A compound as claimed in claim 5, in which R¹ and R¹′ are independently —H, or alkyl with less than 6 carbon atoms; R², R²′, R²⁰, R²⁰′, R³, R³′, R³⁰, R³⁰′, R⁴, R4′, R⁴⁰ and R⁴⁰′ are each independently selected from —H, —OH or -L; and R⁵ and R⁵ are each independently selected from CH₂OH or CH₂L.

7. A compound as claimed in claim 1, in which the label is luminescent, radioactive, detectable by nuclear magnetic resonance (NMR) techniques, or detectable by X-ray photographic techniques.

8. A compound as claimed in claim 1, in which the label is detectable by an in vivo imaging technique.

9. A compound as claimed in claim 1, in which the label comprises a fluorophor, one or more positron emitting nuclei selected from ¹⁸F ⁶⁴Cu and ¹²⁴I, one or more radioactive isotopes selected from ¹⁴C, ³H, ¹²³I and ¹³¹I, one or more NMR-detectable isotopes selected from ¹³C, ²H or ¹⁹F, or an X-ray detectable heavy element with an atomic number of at least 35.

10. A compound as claimed in claim 9, in which the fluorophor is selected from fluoresceins, xanthenes, cyanines, naphthalenes, coumarins, oxadiazoles, pyrenes, oxazines, acridines, arylmethines, Alexa Fluors, tetrapyrroles, and quantum dots.

11. A compound as claimed in claim 1, in which the label is an isotopically enriched analogue of the substrate, being enriched with one or more isotopes selected from ¹³C, ¹⁴C, ²H or ³H.

12. A method for determining the presence of mycobacteria species in an organism or biological sample, the method comprising adding to the organism or biological sample a probe molecule comprising a substrate and a label, which probe molecule can be incorporated into mycobacteria, the presence of mycobacteria being determined by a detector responsive to the presence of the label, optionally after applying a stimulus.

13. A method as claimed in claim 12, in which the probe molecule is capable of being incorporated into the mycolic acid layer of the cell wall of mycobacteria by the action of trehalose transesterase enzymes secreted by the mycobacteria.

14. A method as claimed in claim 12, in which the probe molecule is a compound for labelling mycobacteria, which compound comprises a label and a substrate, which label can be detected by a detector responsive to the presence of the label, optionally after applying a stimulus, characterised by the compound being able to engage with the active site of Antigen 85B (Ag85B) such that it can form simultaneous hydrogen bonds with two or more amino acids in the active site selected from Arg 43, Trp 264, Ser126. His 262 and Leu 42, or the corresponding amino acids in Antigen 85A (Ag85A) or Antigen 85C (Ag85C), at least one of which is with Ser126.

15. A method as claimed in claim 12, in which the probe molecule is added to an organism, and the label is detectable by and detected by an in vivo imaging technique.

16. A method as claimed in claim 12, in which the probe molecule is added to a biological sample selected from sputum, cerebrospinal fluid, pericardial fluid, synovial fluid, ascitic fluid, blood, bone marrow, urine and faeces.

17. A method as claimed in claim 12, in which the mycobacteria are of the species Mycobacteria Tuberculosis.

18. A compound as claimed in claim 4, in which;
R^{a} comprises 6 carbon atoms or less; and/or
R^{b} comprises 6 carbon atoms or less; and/or
Z, Z′ and Z″ at each occurrence is selected from O and S; and/or
W is selected from N and P; and/or
in the definition of E, the Group 16 element is selected from O, S and Se and/or W is selected from N and P and/or V is selected from C and Si.

19. A compound as claimed in claim 5, in which at least one of R⁴⁰ and R⁴⁰′ is selected from OH and SH.

20. A method as claimed in claim 13, in which the trehalose transesterase enzymes secreted by the mycobacteria are one or more of Ag85A, Ag85B and Ag85C.
